(19) United States
(12) Patent Application Publication
(10) Pub. No.: US 2025/0049894 A1
(43) Pub. Date: Feb. 13, 2025

(54) TREATMENT OF NUDT2 MUTATION

(71) Applicant: UNIVERSITÉ DE GENÈVE, Genève (CH)

(72) Inventors: Ramesh PILLAI, Genève (CH); Hao WU, Genève (CH); David HOMOLKA, Genève (CH)

(21) Appl. No.: 18/697,384

(22) PCT Filed: Sep. 28, 2022
(86) PCT No.: PCT/EP2022/076958

§ 371 Date: Mar. 29, 2024

(30) Foreign Application Priority Data
Sep. 30, 2021 (EP) 21200153.1

(51) Int. Cl. A61K 38/46; A01K 67/0276; A61K 31/519; C12Q 1/6883

(52) U.S. Cl. CPC A61K 38/46; A01K 67/0276; A61K 31/519; C12Q 1/6883; C12Y 306/01017; A01K 2217/075; A01K 2227/105; A01K 2267/0306; C12Q 2600/158

(57) ABSTRACT

The disclosure relates to a method of treating Nudt2 disorder in a subject, and a composition for use in said method. The disclosure also relates to an animal model of Nudt2 disorder, and a method of diagnosing a Nudix disorder.

### FIELD OF THE DISCLOSURE

The disclosure relates to a method of treating Nudt2 disorder in a subject, and a composition for use in said method. The disclosure also relates to an animal model of Nudt2 disorder, and a method of diagnosing a Nudix disorder.

### BACKGROUND

Eukaryotic RNA polymerase II transcripts are co-transcriptionally capped at the 5′ end with a N⁷-metylguanosine (m⁷G) moiety that is enzymatically attached to the transcription start site nucleotide. The m⁷G cap structure (cap0) and its further methylated state with an additional 2′-O-methylation on the TSS nucleotide (cap1) are essential for mRNA translation, stability, and identification of the RNAs as ‘self’ by the innate immune system. Removal of the cap by decapping enzymes leads to RNA degradation. Prokaryotic and eukaryotic RNA polymerases can also use cellular metabolites as the initiating nucleotide to generate alternatively capped RNAs. Alternative capping with nicotinamide adenine dinucleotide (NAD) has, for example, been demonstrated.

The Nudix hydrolases are a superfamily of hydrolytic enzymes, named for their ability to cleave nucleoside diphosphates linked to any moiety (x). Nudix enzymes are primarily hydrolases acting on specific metabolite substrates, with different family members having different substrate preferences.

Several Nudix family members (NUDT2, NUDT3, NUDT12, NUDT15, NUDT16, NUDT17, NUDT19, and NUDT20) have been shown to have m⁷G-RNA decapping activity in vitro. Of these, decapping enzyme 2 (DCP2 or NUDT20) is the main m⁷G-RNA decapping activity across all eukaryotes, and is essential for mouse development. Due to their ability to hydrolyse specific metabolite substrates, Nudix enzymes may also be able to decap RNAs that are alternatively capped with their preferred substrate. For example, NUDT12 (which hydrolyses NADH) has been demonstrated to be specialised for decapping NAD-capped RNAs.

Mutations in Nudix enzymes can lead to disorders in humans. For example, homozygous loss-of-function mutations in human NUDT2 causes developmental delay and intellectual disability. This disorder may be known as Nudt2 disorder, or NUDT2 associated syndrome. The underlying molecular basis for Nudt2 disorder is not known, and treatments are not currently available. Means for investigating the molecular basis of, and treatments for, Nudt2 disorder are therefore required, as are treatments themselves.

### SUMMARY OF THE DISCLOSURE

The present inventors have, for the first time, developed an animal model of Nudt2 disorder. Using the animal model, the inventors have determined the physiological relevance of NUDT2. In particular, the inventors have demonstrated that NUDT2 deficiency leads to a dramatic increase in its substrate, the metabolite diadenosine tetraphosphate (Ap4A), in multiple tissues. As a consequence of increased Ap4A, Ap4A-capped RNAs (Ap4A-RNAs) are also increased. The inventors have further demonstrated that increased Ap4A-RNA leads to increased type I interferon (IFN) expression, and a general activation of the type I IFN-dependent gene expression pathway as the common gene expression consequence of Nudt2 disorder.

The present inventors have therefore identified that Nudt2 disorder may be treated by reducing type I interferon (IFN) signalling. The present inventors have also identified that the increase in Nudt2 substrate (Ap4A) or substrate-capped RNA (Ap4A-RNA) associated with Nudt2 disorder may be used as a biomarker for diagnosing Nudt2 disorder. In the same way, an increase in Nudix substrate or substrate-capped RNA may be used as to diagnose disorders of other Nudix enzymes.

The disclosure therefore provides a method of preventing or treating Nudt2 disorder in an individual, the method comprising administering to the individual an agent whose administration reduces type I interferon (IFN) signalling in the individual. The disclosure also provides:
an agent for use in a method of preventing or treating Nudt2 disorder in an individual, wherein the method comprises administering the agent to the individual, and administration of the agent reduces type I IFN signalling in the individual;
an animal model of Nudt2 disorder, wherein the animal lacks expression of functional NUDT2 protein; and
a method of diagnosing a Nudix disorder in an individual, comprising identifying an increased amount of substrate for a Nudix enzyme in a sample obtained from the individual relative to a reference value.

### BRIEF DESCRIPTION OF THE FIGURES

FIG. 1: Loss of Nudt2 causes sub-viability and male infertility in mice. (A) Comparison of phenotypes in human NUDT2 patients and in the Nudt2mouse mutant. (B) Western blot showing expression of NUDT2 in mouse tissues. Expression of PARK7 is used as loading control. (C) Nudt2 mouse mutant is a complete null, and (D) displays sub-viability (male: n=424, female: n=419). (E) Mutant mice have small body-size and weight. (F) Activity of mice (n=8 for each genotype) on a running wheel. The cumulative rotation counts over a 24-hour period is shown. There is no activity during daytime (counting was done every 15 min, 24 h a day, and repeated for 5.5 days). Error bars represent SD. (G) Cartoon showing progression of germ cells through spermatogenesis. Testis histology (stage VII-VIII tubules) shows that all germ cell types are present in the Nudt2KO animals. Stain used is hematoxylin and cosin (H&E). Sp, Pre-leptotene spermatocytes; Ser, Sertoli cells. (H) Electron micrographs showing presence of fully formed sperm in the mutant testis. (I) Sperm do not accumulate in the epididymis. Stain used is hematoxylin and cosin (H&E). (J) One representative testis and epididymis from four individual mice of the indicated genotypes are shown. The major gonadal fat deposit (epididymal white adipose tissue, EWAT) is indicated. (K) Quantification (n=8 for each genotype) of EWAT and brown fat (brown adipose tissue, BAT) using micro-CT. The scans show the different fat deposits overlaid on the skeletal structure. WAT (yellow), BAT (blue) and EWAT (magenta). Error bars represent SD.

FIG. 2: Nudt2 knockout mouse tissues accumulate Ap4A-RNAs. (A) Phenotypes of mouse mutants for eight Nudix enzymes. (B) In vitro RNA decapping activity of Nudix proteins analyzed using cap-labelled m⁷G-RNAs and thin-layer chromatography. NUDT2 can decap m⁷G-RNAs. (C-D) Diadenosine tetraphosphate (Ap4A) synthesis by lysyl tRNA synthetase. (D) Ap4A is hydrolyzed by recombinant NUDT2 into ATP and AMP. Asterisks indicate 32P radioactivity label. (E) Metabolomic analyses show accumulation of Ap4A in Nudt2 knockout (KO) mouse tissues (n=3 for each genotype). (F) Mass spectrometry of polyA+ RNA from Nudt2KOtissues identifies an enrichment of Ap4A. Chemical structures of Ap4A and m7G cap structures on RNA are shown. (G) In vitro RNA decapping activity of Nudix proteins DCP2, NUDT2 and NUDT12 analyzed using cap-labelled Ap4A-RNAs and thin-layer chromatography. Only NUDT2 can decap Ap4A-RNA.

FIG. 3: Type I interferon signalling pathway is activated in Nudt2 knockout tissues. (A) Transcriptomic analyses of multiple Nudt2 knockout (KO) tissues shows that, except for spleen, most tissues do not show dramatic gene expression changes. Type I interferon signaling pathway genes are highlighted. (B) Type I interferon signaling pathway (GO: 0060337) is overrepresented among GO terms associated with upregulated genes. (C) Counts of GO: 0060337 annotated genes which are upregulated in individual tissues of KO mice. (D) Expression changes in individual tissues for the GO: 0060337 annotated genes upregulated in the KO. (E) Western blot showing upregulation of an interferon-stimulated gene, RSAD2 inNudt2KOtissues, especially the spleen. (F) Increased weight of spleen in the Nudt2 KO is indicative of splenomegaly (n=4 for each genotype). Error bars represent SD. (G) Detection of interferon (IFN) a and B in blood serum using ELISA. Mice were bred at our lab facility (n=4 for each genotype) or a commercial facility (n=3 for each genotype). Error bars represent SD. ***p-value<=0.001, **p-value<=0.01,*p-value<=0.05; t-test.

FIG. 4: Non-self Ap4A-RNA triggers type I interferon production in mammalian cells. (A) Preparation of mouse embryonic fibroblasts (MEFs) from mouse embryos. (B) MEFs were left untreated or transfected with in vitro transcribed 50 nt or 200 nt single-stranded RNAs, and cell culture supernatant was collected for detection of interferons using ELISA. Error bars represent SD. RNAs with a 5′ triphosphate (Tri-pho) or m7G cap0 or m7G cap1 or Ap4A structures were used. (C) Ap4A-RNAs were transfected at different concentrations, followed by ELISA to detect interferons. Error bars represent SD.

FIG. 5: Generation of Nudt2 mouse mutant. (A) Multiple tissue Western blot showing expression of NUDT2 in indicated adult mouse tissues. PARK7 levels are used as loading control. P60: Post-natal age of 60 days. (B) Cartoon showing deletion of a genomic region from mouse Nudt2 gene locus, removing sequences that contribute to the catalytic domain. Sequences of the two guide RNAs (gRNAs) used for targeting the Cas9 endoribonuclease are shown. The PAM sequences used by the gRNAs are highlighted (in red). Catalytic domain is highlighted in green. The sequence deletion in the Nudt2−/− knockout mutant is shown. The deletion is expected to result in a frameshift, leading to premature termination of translation (in blue), and causing nonsense-mediated decay (NMD) of the mutant messenger RNA (see FIG. 10A). (C) An ethidium bromide-stained gel showing resolved genomic PCR fragments used for genotyping the mice. Genomic DNA from car-punches were used. Wild-type (+/+), heterozygous (+/−) and homozygous (−/−) genotypes of Nudt2 are indicated. (D) Measured body weight (in grams, g) of animals (n=4) of indicated genotypes. Error bars represent SD. This panel is also placed in FIG. 6A.

FIG. 6: Nudt2 mutant males produce sperm that does not accumulate in the epididymis. (A) Cartoon showing the Nudix domain in mouse NUDT2. Representative testes from wild-type and Nudt2 knockout (KO) male adult mice (four animals each). Measured (n=4) body weights and testis weights are shown. The body weight data is the same as fig. S1D. Error bars represent SD. (B) Histological examination of adult mouse testes showing the presence of all the indicated germ cell types in both wild-type and in the Nudt2 KO testes. Sections are stained with hematoxylin (stains nucleic acids blue) and cosin (stains proteins pink). Scale bar is indicated. (C) Histological examination of different parts of mouse epididymis: initial segments that drain contents from testis into the head/caput, followed by the body/corpus and finally the distal tail/cauda regions. In wild-type mice, most of the sperm is usually seen in the cauda. Sperm is absent in the mutant epididymis. Zooms of mutant cauda showing presence of isolated single sperm are shown below. Stain used is hematoxylin and cosin (H&E). Scale bar is indicated.

FIG. 7: Brown adipose tissue is reduced in the Nudt2 knockout mice. (A) Micro-CT scans of all (n=8 for each genotype) the wild-type (Nudt2+/+) and knockout (Nudt2−/−) animals examined for detecting fat deposits. A composite view (fat) of white adipose tissue (WAT, yellow), epididymal white adipose tissue (EWAT, magenta) and brown adipose tissue (BAT, blue) is shown, while that for EWAT is shown separately. (B) Bar plots show quantification of indicated parameters from 8 biological replicates of each genotype. Absolute values, and that normalized to body weight are shown. Values for biological replicates are separately shown (C) Bar plots show mean values of the indicated parameters. Error bars represent SD. (D) Histological examination of BAT from wild-type (Nudt2+/+) or knockout (Nudt2−/−) mutant mice. Sections are stained with Hematoxylin (stains nucleic acids blue) and cosin (stains proteins pink). Notice the large lipid droplets in the Nudt2 KO BAT. Scale bar (in micrometers, μm) is indicated.

FIG. 8: Measurement of metabolic parameters using LabMaster. Eight animals of each genotype was used for LabMaster experiment, measurements are taken every 15 min, for 24 h a day, and repeated for 5.5 days. Error bars represent SD. (A-D) Measurement of metabolic consumption of oxygen (VO2), and production of carbon dioxide (VCO2) over a period of over 5 days for wildtype (Nudt2+/+) and Nudt2 (Nudt2−/−) mutant adult mice. The dark- and light-periods are indicated with different shading. The values are normalized to total body weight (A and C) or to lean weight (B and D). Lean weight is body weight minus weight of fat (estimated by magnetic resonance imaging, MRI). (E) The ratio of VCO2/VO2, termed the respiratory exchange ratio (RER), provides information about the substrate used for energy production: a value of 1.0 indicates glucose oxidation, while 0.7 represents fat oxidation. (F-G) Energy produced in kilocalories (kcal) normalized to body weight or lean weight (in kcal/hour/gram). The Nudt2 mutant expends more energy. (H) Production of energy (in kilocalories) from fat. (I) General movement (MVT) of the mice in cages do not show any difference. (J-K) Daily water in-take normalized to body weight or lean weight. (L) Cumulative water in-take over several days shows it is same for both genotypes. Food in-take (not shown) was also not different between the two genotypes.

FIG. 9: NUDT2 is specialized for decapping Ap4A-R.VA. (A) Purification of recombinant full-length human and mouse NUDT2. Some of the fractions from the size-exclusion chromatography purification were resolved by SDS-PAGE and stained with Coomassic. Fraction #26 of human NUDT2 was used for experiments. The mouse protein was used for comparison and was found to have identical activity. The 6×-His tagged protein was used for experiments. (B) In vitro RNA decapping activity of NUDT2 analyzed using cap-labelled m7G-RNAs and thin-layer chromatography. The asterisk indicates 32P label. Two different tagged versions of NUDT2 were used. The larger tag of 6×-His-SUMO was found to be reducing the activity. NUDT2 can decap m7G-RNAs by liberating either m7GMP or m7GDP, with the former being in higher amounts. (C) Quality of recombinant full-length human NUDT2 (6×-His-SUMO tagged) and NUDT12 (untagged), while mouse DCP2 protein (95-260 amino acids encompassing only the catalytic domain, untagged). (D) Production of Ap4A from radioactive ATP using recombinant lysyl tRNA synthetase. Reactions (Ap4A mix) were resolved by thin-layer chromatography and is shown to have input ATP and the additional Ap4A. The asterisk indicates 32P label. (E) Metabolomics analyses of mouse tissue lysates from animals of indicated genotypes to detect Gp4A. (F) The Ap4A mix from panel D was further purified using HPLC (see methods) to obtain pure radioactive Ap4A. Hydrolysis of 32P-radiolabelled Ap4A by Nuclease P1 (Nuc. P1), 0.5 unit was used in the third lane, while 1 unit was used in the fourth lane. The asterisk indicates 32P label. Nuclease P1 cleaves the polyphosphate bonds to produce mainly AMP. (G) In vitro RNA decapping activity of human NUDT2 analyzed using cap-labelled Ap4A-RNAs and thin-layer chromatography. (H) In vitro RNA decapping activity of different Nudix proteins analyzed using cap-labelled Ap4A-RNAs and thin-layer chromatography. Proteins were used at different concentrations (in nM). Only NUDT2 can decap Ap4A RNA. The Ap4A RNA was prepared using the Ap4A mix produced in panel D. The proteins used are as in panel C. All experiments in FIG. 2 are done with HPLC-purified radioactive Ap4A from panel F and using 6×His-hNUDT2 protein as in panel A.

FIG. 10: Analysis of Nudt2 mutant tissue transcriptomes. (A) Normalized read counts of Nudt2 in the RNA-seq datasets from the indicated mouse tissues from either wild-type (WT) or Nudt2 knockout (KO) mutant. The overall levels of Nudt2 mRNA are reduced in the KO, consistent with the mutation resulting in nonsense-mediated decay of the mRNA. (B) Number of genes dysregulated in the Nudt2 mutant (KO) mouse tissues when compared to the wild-type (WT). The spleen shows the most profound alternations. (C) Genes whose transcripts are either up- or down-regulated in the Nudt2 mutant. There is very little overall overlap in the gene expression changes, and most changes are specific to each tissue. Interferon stimulated genes (ISGs) are a common class of genes upregulated in all tissues of the KO mutant as shown in FIG. 3. (D) One of the interferon-stimulated gene (ISG) that is consistently upregulated in most tissues is RSAD2. (E) Histological examination of spleen from wild-type and Nudt2 KO adult mice. Sections were stained with hematoxylin and cosin (H&E). Images captured with two different objectives (5× or 20×) are presented. The scale bars are indicated. The white pulp with darkly stained nuclei of lymphoid T and B cells, and red pulp with lightly stained erythrocyte is interspersed between them. The Nudt2 KO spleen shows an increase in number of cells with darkly stained nuclei in the red pulp.

FIG. 11: Sequence of the NUDT2 knockout mouse (A)>NM_025539.2 Mus musculus nudix (nucleoside diphosphate linked moiety X)-type motif 2 (Nudt2), mRNA (SEQ ID NO:1). Underlined nucleotides are deleted in the knockout. Dash-underlined letters are UTRs. (B) Coding sequence after modification (67 bp deletion with frame shift). (C) Deletion comprised in NUDT1 knockout mouse. The deletion is a 67 bp deletion (GAGAGAATGACTTAGAAACAGCCCTGCGAGAGACTCGGGAGGAAACAGGCA TAGAAGCAAGCCAACT; SEQ ID NO: 3) within the hydrolase (catalytic) domain of the Nudix box in exon 3 of Nudt2.

FIG. 12: IFN pathway. Adapted from Platanias, Nature Reviews Immunology, 2005.

FIG. 13: Number of pups born to female partners of IFNR+/NUDT2+ male mice (n=5), IFNR−/NUDT2+ male mice (n=5), IFNR+/NUDT2− male mice (n=5), and IFNR−/NUDT2− male mice (n=4).

FIG. 14: Spleen/body weight (mg/g) in IFNR+/NUDT2+, IFNR−/NUDT2+, IFNR+/NUDT2− and IFNR−/NUDT2− mice.

### BRIEF DESCRIPTION OF THE SEQUENCE LISTING

SEQ ID NO: 1—NM_025539.2 Mus musculus nudix (nucleoside diphosphate linked moiety X)-type motif 2 (Nudt2), mRNA

SEQ ID NO: 2—Coding sequence of the NUDT2 allele in the NUDT2 knockout mouse described in the Examples. The mouse contains a 67 bp deletion in Nudt2, which results in a frame shift.

SEQ ID NO: 3—67 bp deleted from Nudt2 in the NUDT2 knockout mouse described in the Examples.

SEQ ID NO: 4—Sequence within the hydrolase (catalytic) domain of the Nudix box in exon 3 of Nudt2.

SEQ ID NO: 5—amino acid sequence encoded by SEQ ID NO: 4.

SEQ ID NO: 6—Sequence within the hydrolase (catalytic) domain of the Nudix box in exon 3 of Nudt2 (SEQ ID NO: 4) following deletion of SEQ ID NO: 3.

SEQ ID NO: 7—amino acid sequence encoded by SEQ ID NO: 6.

SEQ ID NO:8—gRNA1 used in the preparation of the NUDT2 knockout mouse described in the Examples.

SEQ ID NO:9—gRNA2 used in the preparation of the NUDT2 knockout mouse described in the Examples.

SEQ ID NO: 10—WH-Oligo30 used in the Examples

SEQ ID NO: 11—WH-Oligo31 used in the Examples

SEQ ID NO: 12—WH-Oligo60 used in the Examples

SEQ ID NO: 13—WH-Oligo98 used in the Examples

SEQ ID NO: 14—WH-Oligo-100 used in the Examples

SEQ ID NO: 15—WH-Oligo-421 used in the Examples

SEQ ID NO: 16—WH-Oligo-424 used in the Examples

SEQ ID NO: 17—WH-Oligo-297 used in the Examples

SEQ ID NO: 18—WH-Oligo-298 used in the Examples

SEQ ID NO: 19—WH-Oligo48 used in the Examples

SEQ ID NO: 20—WH-Oligo49 used in the Examples

SEQ ID NO: 21—WH-Oligo-413 used in the Examples

SEQ ID NO: 22—WH-Oligo-414 used in the Examples

SEQ ID NO: 23—WH-Oligo-401 used in the Examples

SEQ ID NO: 24—WH-Oligo-402 used in the Examples

SEQ ID NO: 25—WH-Oligo-436 used in the Examples

SEQ ID NO: 26—WH-Oligo-437 used in the Examples

SEQ ID NO: 27—WH-Oligo-364 used in the Examples

SEQ ID NO: 28—WH-Oligo-365 used in the Examples

SEQ ID NO: 29—WH-Oligo27 used in the Examples

SEQ ID NO: 30—WH-Oligo28 used in the Examples

SEQ ID NO: 31—WH-Oligo95 used in the Examples

SEQ ID NO: 32—WH-Oligo97 used in the Examples

SEQ ID NO: 33—WH-Oligo33 used in the Examples

SEQ ID NO: 34—WH-Oligo34 used in the Examples

SEQ ID NO: 35—WH-Oligo101 used in the Examples

SEQ ID NO: 36—WH-Oligo103 used in the Examples

SEQ ID NO: 37—WH-Oligo36 used in the Examples

SEQ ID NO: 38—WH-Oligo37 used in the Examples

SEQ ID NO: 39—WH-Oligo104 used in the Examples

SEQ ID NO: 40—WH-Oligo106 used in the Examples

SEQ ID NO: 41—WH-Oligo39 used in the Examples

SEQ ID NO: 42—WH-Oligo40 used in the Examples

SEQ ID NO: 43—WH-Oligo107 used in the Examples

SEQ ID NO: 44—WH-Oligo109 used in the Examples

SEQ ID NO: 45—WH-Oligo42 used in the Examples

SEQ ID NO: 46—WH-Oligo43 used in the Examples

SEQ ID NO: 47—WH-Oligo110 used in the Examples

SEQ ID NO: 48—WH-Oligo112 used in the Examples

SEQ ID NO: 49—WH-Oligo45 used in the Examples

SEQ ID NO: 50—WH-Oligo46 used in the Examples

SEQ ID NO: 51—WH-Oligo113 used in the Examples

SEQ ID NO: 52—WH-Oligo115 used in the Examples

SEQ ID NO: 53—Forward (F) primer design template used in the Examples. N represent the gene-specific sequence.

SEQ ID NO: 54—Sequence of the N-terminal 3×FLAG-HA tag.

SEQ ID NO: 55—DNA template used in the Examples.

SEQ ID NO: 56—DNA template used in the Examples.

### DETAILED DESCRIPTION

It is to be understood that different applications of the disclosed methods and products may be tailored to the specific needs in the art. It is also to be understood that the terminology used herein is for the purpose of describing particular embodiments of the disclosure only, and is not intended to be limiting.

All publications, patents and patent applications cited herein, whether supra or infra, are hereby incorporated by reference in their entirety.

### General Definitions

Unless defined otherwise, technical and scientific terms used herein have the same meaning as commonly understood by a person skilled in the art to which this disclosure belongs.

As used in this specification and the appended claims, the singular forms “a”, “an”, and “the” include plural referents unless the content clearly dictates otherwise. Thus, for example, reference to “a polynucleotide sequence” includes “polynucleotide sequences”, reference to “an agent” includes two or more such agents, and the like.

In general, the term “comprising” is intended to mean including but not limited to. For example, the phrase “a method comprising administering to the individual an agent whose administration reduces type I interferon (IFN) signalling in the individual” should be interpreted to mean that method involves administration of the agent, but that the method may also involve administering one or more other substances to the individual.

In some aspects of the disclosure, the word “comprising” is replaced with the phrase “consisting of”. The term “consisting of” is intended to be limiting. For example, the phrase “a method consisting of administering to the individual an agent whose administration reduces type I interferon (IFN) signalling in the individual” should be understood to mean that the method involves only administration of the agent, and no further substances.

The terms “protein” and “polypeptide” are used interchangeably herein, and are intended to refer to a polymeric chain of amino acids of any length.

For the purpose of this disclosure, in order to determine the percent identity of two sequences (such as two polynucleotide or two polypeptide sequences), the sequences are aligned for optimal comparison purposes (e.g., gaps can be introduced in a first sequence for optimal alignment with a second sequence). The nucleotide residues at nucleotide positions are then compared. When a position in the first sequence is occupied by the same nucleotide residue as the corresponding position in the second sequence, then the nucleotides are identical at that position. The percent identity between the two sequences is a function of the number of identical positions shared by the sequences (i.e., % identity=number of identical positions/total number of positions in the reference sequence×100).

Typically the sequence comparison is carried out over the length of the reference sequence. For example, if the user wished to determine whether a given (“test”) sequence has a certain percentage identity to SEQ ID NO: X, SEQ ID NO: X would be the reference sequence. For example, to assess whether a sequence is at least 80% identical to SEQ ID NO: X (an example of a reference sequence), the skilled person would carry out an alignment over the length of SEQ ID NO: X, and identify how many positions in the test sequence were identical to those of SEQ ID NO: X. If at least 80% of the positions are identical, the test sequence is at least 80% identical to SEQ ID NO: X. If the sequence is shorter than SEQ ID NO: X, the gaps or missing positions should be considered to be non-identical positions.

The skilled person is aware of different computer programs that are available to determine the homology or identity between two sequences. For instance, a comparison of sequences and determination of percent identity between two sequences can be accomplished using a mathematical algorithm

### Treatment of Nudt2 Disorder

Disclosed herein is a method of preventing or treating Nudt2 disorder in an individual, the method comprising administering to the individual an agent whose administration reduces type I interferon (IFN) signalling in the individual. Also disclosed herein is an agent for use in a method of preventing or treating Nudt2 disorder in an individual, wherein the method comprises administering the agent to the individual, and administration of the agent reduces type I IFN signalling in the individual.

As explained above and in the Examples, an agent that reduces type I IFN signalling may be used to treat Nudt2 disorder because increased type I IFN expression, and a general activation of the type I IFN-dependent gene expression pathway, is the ultimate consequence of Nudt2 disorder. In particular, loss-of-function mutation of NUDT2 leads to the accumulation of substrate, namely Ap4A. Increased levels of substrate in turn lead to an increased frequency of Ap4A-capped RNA (Ap4A-RNA). As Ap4A-RNA is non-self, type I IFN production is triggered. By interfering with the events leading to production of type I IFN, or inhibiting the downstream consequences of type I IFN production (e.g. activation of the type I IFN-dependent gene expression pathway), clinical signs or symptoms of Nudt2 disorder may be reduced, abolished, stopped from progressing, or prevented from developing.

### Nudt2 Disorder

Nudt2 disorder may be defined as a disorder that is caused by loss-of-function of NUDT2. NUDT2 is a member of the Nudix family of proteins. Nudt2 disorder may otherwise be known as NUDT2 disorder, NUDT2 associated syndrome or NUDT2-related disease, for example. NUDT2-related disease is, for example, disclosed in Yavuz et al. (2018), Clinical Genetics. 2018; 94:393-395.

Nudt2 disorder typically presents as a neurodevelopmental disorder. Symptoms may, for example, include developmental delay, intellectual disability, and/or motor dysfunction. Developmental delay may, for example, manifest as smaller body size and or lower body weight than expected at a given stage of development. Motor dysfunction may, for example, manifest as weakness of the extremities and/or frequent falls. Motor dysfunction may, for example, arise as a result of a polyneuropathy, such as a sensorimotor polyneuropathy.

Loss-of-function of NUDT2 may, for example, be caused by one or more mutations in the Nudt2 gene. The one or more mutations may, for example, comprise (i) one or more deletions, (ii) one or more insertions, and/or (iii) one or more substitutions. For instance, the one or more mutations may comprise (i); (ii); (iii); (i) and (ii); (i) and (iii); (ii) and (ii); or (i), (ii) and (iii). The one or more mutations may, for example, comprise or consist of a homozygous frameshift variant c.186delA (p.A63Qfs*3). c.186delA (p.A63Qfs*3) is described in Diaz et al. (2020), Annals of Clinical and Translational Neurology, 7 (11): 2320-2325. The one or more mutations may, for example, comprise or consist of a homozygous nonsense variant c.34C>T (p.Arg12*). c.34C>T (p.Arg12*) is described in Yavuz et al. (2018), Clinical Genetics. 2018; 94:393-395.

Accordingly, the method of preventing or treating Nudt2 disorder in an individual may alternatively be defined as, for example, a method of preventing or treating a neurodevelopmental disorder in an individual that has loss-of-function of NUDT2. The method of preventing or treating Nudt2 disorder in an individual may alternatively be defined as, for example, a method of preventing or treating (i) developmental delay, (ii) intellectual disability, and/or (iii) motor dysfunction in an individual that has loss-of-function of NUDT2. For instance, the method of preventing or treating Nudt2 disorder in an individual may alternatively be defined as a method of preventing or treating (i); (ii); (iii); (i) and (ii); (i) and (iii); (ii) and (iii); or (i), (ii) and (iii) in an individual that has loss-of-function of NUDT2. In any case, the individual may comprise a Nudt2 gene that comprises one or more loss-of-function mutations. Exemplary loss-of-function mutations are described above.

### Individual

The individual may be an individual that has Nudt2 disorder. The individual may show signs of Nudt2 disorder, such as developmental delay, intellectual disability, and/or motor dysfunction. In this case, administration of the agent may treat the Nudt2 disorder. That is, administration of the agent may reduce or abolish the clinical signs or symptoms of Nudt2 disorder, or stop them from progressing.

The individual may be an individual that is at risk of developing Nudt2 disorder. For example, the individual may have a mutation that causes loss-of-function of NUDT2, but not yet show clinical signs or symptoms of Nudt2 disorder. In other words, the individual may be presymptomatic. In this case, administration of the agent may prevent the development of clinical signs or symptoms of Nudt2 disorder.

The individual may, for example, be a mammal. Preferably, the mammal is a human.

The individual may be of any age. For example, the individual may be a juvenile. The individual may, for example, be an adult.

The individual may, for example, be a prenatal individual. In other words, the individual may be in utero. That is, the individual may be a foetus. The prenatal individual may, for example, be presymptomatic. In other words, the prenatal individual may have a mutation that causes loss-of-function of NUDT2, but not yet show signs of Nudt2 disorder.

### Agent

In order to prevent or treat Nudt2 disorder, the individual is administered an agent whose administration reduces type I IFN signalling in the individual. Administration of the agent may, for example, reduce type I IFN signalling in the individual relative to type I IFN signalling in the individual prior to administration of the agent.

The agent may be any agent whose administration reduces type I IFN signalling in the individual. Such agents are known in the art. For instance, as set out below, the agent may be an inhibitor of the JAK-STAT pathway. Inhibitors of the JAK-STAT pathway are known in the art and include, for example, ruxolitinib. As set out below, the agent may for instance be an inhibitor of type I IFN binding to IFNAR. Such inhibitors are known in the art and include, for example, IFNAR1-specific antibodies such as anifrolumab or MAR1-5A3.

Type I IFN signalling refers to signalling by one or more type I IFNs. Type I IFNs are proteins that function in regulating the activity of the immune system. Type I IFNs include IFN-α, IFN-β, IFN-κ, IFN-δ, IFN-ε, IFN-τ, IFN-ω and IFN-ζ. Administration of the agent may reduce signalling by IFN-α, IFN-β, IFN-κ, IFN-δ, IFN-ε, IFN-τ, IFN-ω and/or IFN-ζ, in any combination. Preferably, administration of the agent reduces IFN-α and/or IFN-β signalling in the individual.

Reduced type I IFN signalling may refer, for example, to a reduced ability of the type I IFN to signal in the individual. In other words, the signalling activity of one or more type I IFN may be reduced. Reduced type I IFN signalling may refer, for example, to a reduced amount of the type I IFN in the serum of the individual. The agent may reduce type I IFN signalling in any one or more of these ways, as described in detail below.

Methods for measuring the signalling activity of a type I IFN are known in the art. Such methods may include, for example; assays for the binding of the type I IFN to its receptor; assays for the activation of signalling pathways (e.g. the JAK-STAT pathway) downstream of receptor binding; and assays for the expression of type I IFN-dependent genes and/or their corresponding proteins (e.g. RT-qPCR, RT-PCR, RNA-seq, Northern blotting, SAGE, DNA microarrays, tilling arrays; in situ hybridization, Western blotting, enzyme-linked immunosorbent assay (ELISA), mass spectrometry). Methods for measuring the amount of type I IFN in serum are also known and include, for example, ELISA.

Type I IFN signalling may be reduced by, for example, at least 30%. In other words, administration of the agent may reduce the signalling activity of one or more type I IFN and/or the amount of one or more type I IFN in the serum by at least 30%. Administration of the agent may, for example, reduce type I IFN signalling by at least 35%, at least 40%, at least 45%, at least 50%, at least 55%, at least 60%, at least 65%, at least 70%, at least 75%, at least 80%, at least 85%, at least 90%, at least 95%, at least 98%, at least 99%. In other words, administration of the agent may reduce the signalling activity of one or more type I IFN and/or the amount of one or more type I IFN in the serum by at least 35%, at least 40%, at least 45%, at least 50%, at least 55%, at least 60%, at least 65%, at least 70%, at least 75%, at least 80%, at least 85%, at least 90%, at least 95%, at least 98%, at least 99%. Administration of the agent may reduce type I IFN signalling by, for example, 30% to 99%, such as 35% to 95%, 40% to 90%, 45% to 85%, 50% to 80%, 55% to 75%, or 60% to 70%. That is, administration of the agent may reduce the signalling activity of one or more type I IFN and/or the amount of one or more type I IFN in the serum by 30% to 99%, such as 35% to 95%, 40% to 90%, 45% to 85%, 50% to 80%, 55% to 75%, or 60% to 70%.

Preferably, administration of the agent reduces type I IFN signalling by at least 30% relative to type I IFN signalling in the individual prior to administration of the agent. That is, administration of the agent may reduce the signalling activity of one or more type I IFN by at least 30% relative to the signalling activity of the one or more type I IFN in the individual prior to administration of the agent. Administration of the agent may reduce the amount of one or more type I IFN in the serum by at least 30% relative to the amount of the one or more type I IFN in the serum of the individual prior to administration of the agent. Administration of the agent may, for example, reduce type I IFN signalling by at least 35%, at least 40%, 45%, at least 50%, at least 55%, at least 60%, at least 65%, at least 70%, at least 75%, at least 80%, at least 85%, at least 90%, at least 95%, at least 98%, at least 99% relative to type I IFN signalling in the individual prior to administration of the agent. For instance, administration of the agent may reduce the signalling activity of one or more type I IFN in the individual by at least 35%, at least 40%, 45%, at least 50%, at least 55%, at least 60%, at least 65%, at least 70%, at least 75%, at least 80%, at least 85%, at least 90%, at least 95%, at least 98%, at least 99% relative to the signalling activity of the one or more type I IFN in the individual prior to administration of the agent. Administration of the agent may, for example, reduce the amount of one or more type I IFN in the serum by at least 35%, at least 40%, 45%, at least 50%, at least 55%, at least 60%, at least 65%, at least 70%, at least 75%, at least 80%, at least 85%, at least 90%, at least 95%, at least 98%, at least 99% relative to the amount of the one or more type I IFN in the serum of the individual prior to administration of the agent. “Prior to administration of the agent” may mean immediately prior to administration of the agent. Alternatively, “prior to administration of the agent” may mean a number of hours (e.g. 1 to 24 hours, such as 6 to 18 hours, 10 to 12 hours, 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours 19 hours, 20 hours, 21 hours, 22 hours, 23 hours or 24 hours) prior to administration of the agent, or a number of days (e.g. 1 to 14 days, such as 2 to 10 days, 3 to 7 days, 1 day, 2 days, 3 days, 4 days, 5 days, 6 days or 7 days) prior to administration of the agent.

In any of the aspects described herein, the administration of the agent may reduce type I IFN signalling by 100%. In other words, the nucleic acid silencing molecule may completely eliminate type I IFN signalling. The agent may, for example, completely eliminate the signalling activity of one or more type I IFN (i.e. reduce the signalling activity of the one or more type I IFN by 100%). The agent may, for example, completely eliminate one or more type I IFN from the serum (i.e. reduce the amount of the one or more type I IFN in the serum by 100%).

To reduce type I IFN signalling, the agent may inhibit the signalling activity of one or more type I IFN. The agent may, for example, inhibit the signalling activity of one or more of IFN-α, IFN-β, IFN-κ, IFN-δ, IFN-ε, IFN-τ, IFN-ω and IFN-ζ. For instance, the agent may inhibit the signalling activity of IFN-α and/or IFN-β. Inhibition of the signalling activity of a type I IFN may refer to reducing the ability of the type I IFN to exert one or more downstream effects. Such downstream effects may, for example, include activation of type I IFN-dependent gene expression, and/or intermediate signalling events that activate type I IFN-dependent gene expression. Accordingly, administration of the agent may reduce activation of type I IFN-dependent gene expression. Administration of the agent may inhibit or block type I IFN-induced signalling events that activate type I IFN-dependent gene expression.

Type I IFN-induced signalling events that activate type I IFN-dependent gene expression may, for example, include binding of the type I IFN to its receptor (the IFN-α receptor, IFNAR). Type I IFN-induced signalling events that activate type I IFN-dependent gene expression may, for example, include signalling via a pathway activated by binding of a type I IFN to IFNAR. Thus, administration of the agent may inhibit or block binding of the type I IFN to IFNAR. Administration of the agent may inhibit or block a pathway activated by binding of a type I IFN to IFNAR. Administration of the agent may inhibit or block binding of the type I IFN to IFNAR, and inhibit or block a pathway activated by binding of a type I IFN to IFNAR. The agent may, therefore, be an agent that inhibits or blocks binding of the type I IFN to IFNAR, and/or inhibits or blocks a pathway activated by binding of a type I IFN to IFNAR. For instance, the agent may be an IFNAR1-specific antibody such as MAR1-5A3.

Pathways activated by binding of a type I IFN to IFNAR are known in the art. Such pathways include the JAK-STAT signalling pathway. When Type I IFN binds to IFNAR, two members of the Janus activating kinase (JAK) family are phosphorylated and activated, namely Tyk2 and JAK1. Tyk2 and JAK1 then phosphorylate members of the signal transducers and activators of transcription (STAT) family, in particular STAT1α (STAT91), STAT1β (STAT84) and STAT2 (STAT113). These phosphorylated STATs disengage from the receptor and bind to the cytoplasmic protein p48. The resultant complex then enters the nucleus and directly binds to regulatory regions upstream of type I IFN-dependent genes. In this way, type I IFNs regulated the expression of IFN-dependent genes. The agent may inhibit or block any point in this JAK-STAT pathway. For example, the agent may inhibit or block JAK phosphorylation, such as phosphorylation of Tyk2 and/or JAK1. The agent may inhibit or block STAT phosphorylation, such as phosphorylation of (i) STAT1α (STAT91), (ii) STAT1β (STAT84) and/or (iii) STAT2 (STAT113). For example, the agent may inhibit or block phosphorylation of (i); (ii); (iii); (i) and (ii); (i) and (iii); (ii) and (iii); or (i), (ii) and (iii). The agent may inhibit or block STAT binding to p48. The agent may block the binding of STAT-p48 complex to regulatory regions upstream of type I IFN-dependent genes.

Inhibitors of the JAK-STAT pathway are known in the art and include, for example, ruxolitinib. Ruxolitinib is an FDA-approved JAK inhibitor with selectivity for JAK1 and JAK2. The agent may, therefore, be ruxolitinib. Other known inhibitors of the JAK-STAT pathway include, for example, ruxolitinib, tofacitinib, oclacitinib, baricitinib, peficitinib, upadacitinib, fedratinib, delgocitinib, filgotinib, abrocitinib, pacritinib, deucravacitinib, cerdulatinib, gandotinib, lestaurlinib, and momelotinib. Therefore, the agent may, for example, be tofacitinib, oclacitinib, baricitinib, peficitinib, upadacitinib, fedratinib, delgocitinib, filgotinib, abrocitinib, pacritinib, deucravacitinib, cerdulatinib, gandotinib, lestaurlinib, or momelotinib.

Alternatively or additionally, the agent may reduce type I IFN signalling by reducing the amount of one or more type I IFN in the serum of the individual. The agent may, for example, reduce the amount of one or more of IFN-α, IFN-β, IFN-κ, IFN-δ, IFN-ε, IFN-τ, IFN-ω and IFN-ζ the serum of the individual. For instance, the agent may reduce the amount of IFN-α and/or IFN-β in the serum of the individual. Methods for determining the amount of a type I IFN are known in the art and include, for example, an enzyme-linked immunosorbent assay (ELISA).

The agent that reduces the amount of one or more type I IFN in the serum of the individual may do so by destroying the type I IFN, or promoting destruction of the type I IFN. For example, the agent may render type I IFN unstable in the serum.

The agent that reduces the amount of one or more type I IFN in the serum of the individual may do so by reducing or blocking the production of type I IFN. For example, the agent may reduce or block transcription of the gene encoding the type 1 IFN, such that the amount of mRNA encoding the type I IFN is reduce. The agent may reduce or block translation of mRNA encoding the type I IFN, such that the amount of type I IFN protein is reduced. The agent may, for example, be a nucleic acid silencing molecule that reduces expression of the type I IFN. The nucleic acid silencing molecule may, for example, be an antisense oligonucleotide, a small interfering RNA (siRNA), a short hairpin RNA (shRNA), a microRNA (miRNA), or a CRISPR guide RNA.

The agent that reduces the amount of one or more type I IFN in the serum of the individual may do so by reducing one or more stimulus for type I IFN production. As explained above and in the Examples, loss-of-function of NUDT2 results in an increase of its substrate, Ap4A. Increased Ap4a promotes the formation of Ap4A-capped RNA. Ap4A-capped RNA is non-self, so is seen as “foreign” by the immune system. Increased Ap4A-capped RNA therefore stimulates the production of type I IFN. A stimulus for type I IFN production may therefore be reduced by promoting hydrolysis of AP4A and/or promoting decapping of Ap4A-capped RNA. Thus the agent may, for example, promote hydrolysis of diadenosine tetraphosphate (Ap4A). The agent may, for example, promote decapping of Ap4A-capped RNA. The agent may, for example, promote hydrolysis of Ap4A and decapping of Ap4A-capped RNA.

Agents that promote hydrolysis of Ap4A and/or decapping of Ap4A-capped RNA are known in the art. Examples include enzymes that promote hydrolysis of Ap4A and/or decapping of Ap4A-capped RNA, such as functional NUDT2. A gene therapy approach may be used to introduce such enzymes into the individual. Accordingly, the agent may comprise a polynucleotide sequence that encodes an enzyme that promotes hydrolysis of Ap4A and/or decapping of Ap4A-capped RNA. The enzyme may, for example, be functional NUDT2 (i.e. wild type NUDT2). The enzyme may, for example, be a variant of wild type NUDT2 that retains hydrolase activity. The variant may, for example, have at least 75% (such as at least 80%, at least 85%, at least 90%, at least 95%, at least 96%, at least 97%, at least 98%, or at least 99%) sequence identity to the amino sequence of wild type NUDT2. Assays for hydrolase activity are known in the art. For example, the production of ATP and AMP from Ap4A may be measured, as described in the Examples.

The polynucleotide sequence may, for example, comprise DNA. The polynucleotide sequence may, for example, comprise RNA. The polynucleotide sequence may, for example, comprise DNA and RNA. The polynucleotide sequence may be comprised in a vector. The vector may, for instance, be a viral vector. The viral vector may, for example, be an adenoviral vector, a retroviral vector, or a lentiviral vector. Alternatively, the polynucleotide sequence may be administered in naked form, for instance using electroporation, a gene gun, sonoporation, or hydrodynamic delivery. The polynucleotide sequence may be administered in a lipoplex, a liposome, a polymersome, a polyplex, or a dendrimer. The polynucleotide sequence may be conjugated to an inorganic nanoparticle, or a cell-penetrating peptide.

### Administration

The agent may be administered by any route. Suitable routes include, but are not limited to, the intravenous, intrathecal, intracerebral ventricular, intramuscular, intraperitoneal, subcutaneous, intradermal, transdermal and oral/buccal routes.

When the individual is a prenatal individual, the agent may be administered to the individual in utero. In other words, the agent may be administered directly to the individual while the individual is in their mother's uterus. The agent may be one that is capable of crossing the placenta. In this case, the agent may be administered to the individual by administering it to the mother of the individual.

The agent may be comprised in a composition that comprises a physiologically acceptable carrier or diluent. Typically, such compositions are prepared as liquid suspensions of the agent. The agent may be mixed with an excipient which is pharmaceutically acceptable and compatible with the active ingredient. Suitable excipients are, for example, water, saline, dextrose, glycerol, of the like and combinations thereof. In addition, if desired, the pharmaceutical compositions may contain minor amounts of auxiliary substances such as wetting or emulsifying agents, and/or pH buffering agents.

The agent is administered in a manner compatible with the dosage formulation and in such amount will be therapeutically effective. The quantity to be administered depends, for example, on the subject to be treated, the nature of the Nudt2 disorder (e.g. symptomatic or presymptomatic), and so on. Precise amounts of the agent required to be administered may depend on the judgement of the practitioner and may be peculiar to each subject.

### Animal Model

Disclosed herein is an animal model of Nudt2 disorder, wherein the animal lacks expression of functional NUDT2 protein. As set out in the Examples, the inventors have demonstrated for the first time that lack of expression of functional NUDT2 protein in the animal confers a phenotype that is shared with human patients having Nudt2 disorder. In particular, animals lacking expression of functional NUDT2 protein have a smaller body size and lower body weight than their NUDT2-sufficient contemporaries, mirroring the developmental delay in human patients with NUDT2 disorder. Animals lacking expression of functional NUDT2 protein also show reduced running activity, mirroring the motor dysfunction seen in human patients with NUDT2 disorder.

Preferably, the animal is a mammal, such as a non-human mammal. More preferably, the animal is a rodent, such as a mouse, rat or hamster. Most preferably, the animal is a mouse. However, the animal may be a non-human mammal other than a rodent, such as a ferret.

Preferably, the animal lacking expression of functional NUDT2 protein lacks expression of NUDT2 protein. That is, the animal may express no NUDT2 protein. The animal may express undetectable levels of NUDT2 protein. The animal may, for example, be a NUDT2 knock-out animal. That is, the animal may comprise one or more non-functional Nudt2 allele. The animal may, for example, comprise one non-functional Nudt2 allele, i.e. be a heterozygous knock-out. Preferably, the animal comprises two non-functional Nudt2 alleles, i.e. is a homozygous knock-out.

The one or more non-functional Nudt2 allele may lack all or part of the Nudt2 gene. In other words, all or part of the Nudt2 gene may be deleted in order to render it non-functional. For instance, the one or more non-functional Nudt2 allele may comprise a deletion of one or more (such as two or more, three or more, four or more, five or more, or six) Nudt2 exons. The one or more non-functional Nudt2 allele may, for example, comprise a deletion in one or more (such as two or more, three or more, four or more, five or more, or six) Nudt2 exons. For example, the one or more non-functional Nudt2 allele may comprise a deletion in or of (i) exon 1, (ii) exon 2, (iii) exon 3, (iv) exon 4, (v) exon 5, or (vi) exon 6 of the Nudt2 gene. For instance, the one or more non-functional Nudt2 allele may comprise a deletion in or of: (i); (ii); (iii); (iv); (v); (vi); (i) and (ii); (i) and (iii); (i) and (iv); (i) and (v); (i) and (vi); (ii) and (iii); (ii) and (iv); (ii) and (v); (ii) and (vi); (iii) and (iv); (iii) and (v); (iii) and (vi); (iv) and (v); (iv) and (vi); (v) and (vi); (i), (ii) and (iii); (i), (ii) and (iv); (i), (ii) and (v); (i), (ii) and (vi); (i), (iii) and (iv); (i), (iii) and (v); (i), (iii) and (vi); (i), (iv) and (v); (i), (iv) and (vi); (i), (v) and (vi); (ii), (iii) and (iv); (ii), (iii) and (v); (ii), (iii) and (vi); (ii), (iv) and (v); (ii), (iv) and (vi); (ii), (v) and (vi); (iii), (iv) and (v); (iii), (iv) and (vi); (iii), (v), (vi); (iv), (v) and (vi); (i), (ii), (iii) and (iv); (i), (ii), (iii and (v); (i), (ii), (iii) and (vi); (i), (ii), (iv) and (v); (i), (ii), (iv) and (vi); (i), (ii), (v) and (vi); (i), (iii), (iv) and (v); (i), (iii), (iv) and (vi); (i), (iii), (v) and (vi); (i), (iv), (v) and (vi); (ii), (iii), (iv) and (v); (ii), (iii), (iv) and (vi); (ii), (iii), (v) and (vi); (ii), (iv), (v) and (vi); (iii), (iv), (v) and (vi); (i), (ii), (iii), (iv) and (v); (i), (ii), (iii), (iv) and (vi); (i), (ii), (iii), (v) and (vi); (i), (ii), (iv), (v) and (vi); (i), (iii), (iv) and (v), (vi); (ii), (iii), (iv), (v) and (vi); or (i), (ii), (iii), (iv), (v), and (vi).

Preferably, the one or more non-functional Nudt2 allele comprises a deletion in exon 3 of the Nudt2 gene. Exon 3 of the Nudt2 gene encodes the critical hydrolytic residues of NUDT2, which are known as the Nudix box. The Nudix box is comprised in the Nudix hydrolase domain. Preferably, the deletion removes the Nudix box and/or the Nudix hydrolase domain. That is, the deletion may comprise or consist of deletion of the Nudix box. The deletion may comprise or consist of deletion of the Nudix hydrolase domain.

The deletion may be of any size. For instance, the deletion may be from 1 to 2000 bp in length. The deletion may, for example, be from 100 to 1900, 200 to 1800, 300 to 1700, 400 to 1600, 500 to 1500, 600 to 1400, 700 to 1300, 800 to 1200, 900 to 1100, or about 1000 bp in length. Preferably, the deletion is from 1 to 100 bp in length, such as 50 to 75 bp in length. The deletion may, for example, be around 67 bp in length, such as 65 bp, 66 bp, 67 bp, 68 bp, 69 bp, or 70 bp in length).

The deletion may, for example, result in a frameshift mutation. The deletion may, for example, result in a premature termination codon. The deletion may result in a frameshift mutation that creates a premature termination codon. Generation of a premature termination codon may result in removal of mutant transcript for instance via nonsense-mediated decay.

Preferably, the deletion is around 67 bp in length and is exon 3 of the Nudt2 gene. Preferably, the deletion is around 67 bp in length, is exon 3 of the Nudt2 gene, and results in a frameshift mutation. Preferably, the deletion is around 67 bp in length, is exon 3 of the Nudt2 gene, results in a frameshift mutation, and creates a premature termination codon. Preferably, the deleted sequence has the sequence of SEQ ID NO: 3. Preferably, the coding sequence of Nudt2 following the deletion has the sequence of SEQ ID NO: 2.

Methods for generating animals that lack expression of a particular protein are known in the art. For example, a CRISPR-mediated approach may be used to knock out the gene encoding the protein. In this approach, a single-cell embryo may be injected with guide RNAs designed to flank a region in the gene. The guide RNAs direct a DNA endonuclease (e.g. Cas9) to delete the flanked region. The deletion is then present throughout the animal that develops from the embryo. Alternatively, a targeting vector that recombines with the target gene may be introduced to embryonic stem (ES) cells. Recombination may knock out a copy of the target gene. ES cells that have successfully incorporated the targeting vector may be selected, and injected into a developing embryo in order to produce a chimeric animal. The chimeric animal can be bread to generate animals containing the knock-out in their germline. mutagenesis, for example by using targeting vector that recombines with the gene. In the present disclosure, the animal model may be generated using any known method. Preferably, a CRISPR-mediated approach is employed to generate the animal model.

The animal lacking expression of functional NUDT2 protein may express a non-functional NUDT2 protein. In other words, the animal may express a mutant NUDT2 that lacks hydrolase ability, such as the ability to hydrolyse ApA4. The animal may express a mutant NUDT2 that has reduced hydrolase ability, such as the ability to hydrolyse ApA4. The hydrolase activity may be reduced by, for example, at least 50%, at least 60%, at least 70%, at least 80%, at least 90%, at least 95%, at least 96%, at least 97%, at least 98% or at least 99% compared to wild type NUDT2. Hydrolase activity may be determined by measuring the ability of NUDT2 to hydrolyse ApA4. Lack of, or reduced, hydrolase activity may be conferred by one or more mutations (such as two or more, three or more, four or more, five or more, 10 or more, 20 or more, or 50 or more) in the Nudt2 gene. Each of the one or more mutations may independently be a deletion, substitution or insertion. The substitution may, for example, be a non-conservative substitution. The one or more mutation may, for example, comprise one or more mutations in the Nudix box. The one or more mutation may, for example, comprise one or more mutations in the Nudix hydrolase domain.

### Method of Diagnosis

The present disclosure provides a method of diagnosing a Nudix disorder in an individual. The method comprises identifying an increased amount of substrate for a Nudix enzyme in a sample obtained from the individual relative to a reference value. The method is advantageous because measuring the amount of substrate in a sample from the individual is significantly easier than identifying a loss-of-function mutation in the Nudix enzyme.

The Nudix disorder may be defined as a disorder that is caused by loss-of-function of a Nudix enzyme. The Nudix enzyme may be any Nudix enzyme. For example, the Nudix enzyme may be MTH1, NUDT2, NUDT3, NUDT4, NUDT5, NUDT6, NUDT7, NUDT8, NUDT9, NUDT10, NUDT11, NUDT12, NUDT13, NUDT14, NUDT15, NUDT16, NUDT17, NUDT18, NUDT19, DCP2, NUDT21 or NUDT22. Preferably, the Nudix disorder is Nudt2 disorder and the Nudix enzyme is NUDT2.

As set out above, different Nudix enzymes have different substrate preferences. The substrate that is identified in an increased amount may be any substrate for a Nudix enzyme. The Nudix enzyme may be a Nudix enzyme whose loss-of-function causes the Nudix disorder. Preferably, the Nudix enzyme is NUDT2 and the substrate is Ap4A or Ap4A-capped RNA.

The Nudix enzyme may be MTH1 and the substrate may be 2-OH-ATP, 2-OH-dATP, 5-Iodo-dCTP, 6-me-thio-GTP, 8-oxo-dGTP, 8-oxo-GTP, dATP, dCTP, dGTP, N2-me-dGTP, 5-Iodo-dCTP, 2-OH-ATP, 2-OH-dATP, 5-me-dCTP, or 8-oxo-dGTP, or RNA capped therewith. The Nudix enzyme may be NUDT3 and the substrate may be Ap6A, or RNA capped therewith. The Nudix enzyme may be NUDT5 and the substrate may be beta-NADH, or RNA capped therewith. The Nudix enzyme may be NUDT12 and the substrate may be NADH or NAD+, or RNA capped therewith. The Nudix enzyme may be NUDT14 and the substrate may be ADP-glucose, ADP-ribose, or beta-NADH, or RNA capped therewith. The Nudix enzyme may be NUDT15 and the substrate may be 5-me-dCTP, 6-thio-dGTP, 6-thio-GTP, 8-oxo-dGTP, dCTP, dGTP, dTTP, or dUTP, or RNA capped therewith. The Nudix enzyme may be NUDT18 and the substrate may be 5-me-dCTP, 5-Fluoro-dUTP, 6-me-thio-GTP, 8-oxo-dGTP, 8-oxo-dGDP, 8-oxo-GTP, ADP, dCDP, dTTP, GDP, or TDP, or RNA capped therewith.

The reference value may, for example, reflect the amount of the substrate in a sample from a healthy individual. The healthy individual may be an individual that does not have the Nudix disorder. The reference value may, for example, reflect the average (e.g. mean) amount of substrate present in samples from a cohort of healthy individuals. Each member of the cohort may be an individual that does not have the Nudix disorder.

The amount of substrate in a sample may be measured using routine metabolomics methods. Exemplary techniques are provided in Example 1, and include liquid chromatography-mass spectrometry (LC-MS).

The amount of substrate in the sample from the individual may, for example, be increased by about 1 to 100 fold compared to the reference value. For instance, the amount of substrate in the sample from the individual may be increased by about 2 fold, 5 fold, 10 fold, 20 fold, 50 fold, 75 fold, 10 to 90 fold, 20 to 80 fold, 30 to 70 fold, or 40 to 60 fold.

For example, when the Nudix disorder is Nudt2 disorder (and the Nudix enzyme is NUDT2), the substrate may be Ap4A. The amount of Ap4A may be increased by about 2 to 20 fold, such as 3 to 19 fold, 4 to 18 fold, 5 to 17 fold, 6 to 16 fold, 7 to 15 fold, 8 to 14 fold, 9 to 13 fold, 10 to 12 fold or 11 fold compared to the reference value. For instance, the amount of Ap4A may be increased by about 3 to 12 fold compared to the reference value. For example, the amount of Ap4A may be increased by about 4 to 11 fold, 5 to 10 fold, 6 to 9 fold, or 7 to 8 fold compared to the reference value. The amount of Ap4A may be increased by about 3, 4, 5, 6, 7, 8, 9, 10, 11 or 12 fold compared to the reference value.

When the Nudix disorder is Nudt2 disorder (and the Nudix enzyme is NUDT2), the substrate may be Ap4A-capped RNA. The amount of Ap4A-capped may be increased by about 2 to 20 fold, such as 3 to 19 fold, 4 to 18 fold, 5 to 17 fold, 6 to 16 fold, 7 to 15 fold, 8 to 14 fold, 9 to 13 fold, 10 to 12 fold or 11 fold compared to the reference value. For instance, the amount of Ap4A-capped RNA may be increased by about 5 to 15 fold compared to the reference value. example, the amount of Ap4A-capped RNA may be increased by about 6 to 14 fold, 7 to 13 fold, 8 to 12 fold, 9 to 11 fold, or 10 fold compared to the reference value. The amount of Ap4A-capped RNA may be increased by about 35, 6, 7, 8, 9, 10, 11, 12, 13, 14 or fold compared to the reference value. In some aspects, the reference value may correspond to a level of about one Ap4A-capped RNA per 1000 mRNAs. In some aspects, the amount of Ap4A-capped RNA in the sample obtained from the individual may correspond to a level or about 50 Ap4A-capped RNAs per 1000 mRNAs to about 100 Ap4A-capped RNAs per 1000 mRNAs, such as about 70 Ap4A-capped RNAs per 1000 mRNAs, about 75 Ap4A-capped RNAs per 1000 mRNAs, about 80 Ap4A-capped RNAs per 1000 mRNAs, about 85 Ap4A-capped RNAs per 1000 mRNAs, about 90 Ap4A-capped RNAs per 1000 mRNAs, or about 95 Ap4A-capped RNAs per 1000 mRNAs. For example, the amount of Ap4A-capped RNA in the sample obtained from the individual may correspond to a level or about 83 Ap4A-capped RNAs per 1000 mRNAs.

The sample from the test individual may, for example, be a blood sample. The sample may comprise a serum sample or a plasma sample. The sample from the test individual may, for example, be a tissue sample. The tissue sample may be derived from any tissue, such as skeletal muscle, skin, liver, kidney, GI tract, lung, heart and so on. The sample(s) from the healthy individuals(s) may, for example, be a blood sample. The sample may comprise a serum sample or a plasma sample. The sample(s) from the healthy individual(s) may, for example, be a tissue sample. The tissue sample may be derived from any tissue, such as skeletal muscle, skin, liver, kidney, GI tract, lung, heart and so on.

Following identification of an increased amount of Nudix substrate in the sample, the method may further comprise the step of identifying one or more loss-of-function mutations in a nucleic acid sequence encoding the Nudix enzyme in the individual. This step may be performed to confirm the diagnosis of the Nudix disorder. The one or more loss-of-function mutations may result in the production of a dysfunctional Nudix enzyme. The one or more loss-of-function mutations may result in a lack of expression of the Nudix enzyme. The nucleic acid sequence comprising the one or more loss-of-function mutations is preferably a DNA sequence, and the DNA sequence is preferably comprised in the gene encoding the Nudix enzyme. The one or more loss-of-function mutation mutations may, for example, comprise one or more point mutations. For instance, the one or more loss-of-function mutation mutations may, for example, comprise (i) one or more deletions, (ii) one or more insertions, and/or (iii) one or more substitutions. For instance, the one or more loss-of-function mutations may comprise (i); (ii); (iii); (i) and (ii); (i) and (iii); (ii) and (ii); or (i), (ii) and (iii). In any case, the one or more loss-of-function mutation mutations may comprise a truncating mutation. The one or more loss-of-function mutations (such as the one or more truncating mutations) may result in the production of a dysfunctional Nudix enzyme, or in lack of expression of the Nudix enzyme. Methods for identifying the one or more loss-of-function mutations are known in the art and include, for example, exome sequencing and targeted sequencing of the relevant Nudix gene.

The following Examples illustrate the invention.

### EXAMPLES

### Example 1

Eukaryotic RNA polymerase II transcripts are co-transcriptionally capped at the 5′ end with a N⁷-metylguanosine (m⁷G) moiety that is enzymatically attached to the transcription start site nucleotide (1). The m⁷G cap structure (cap0) and its further methylated state with an additional 2′-O-methylation on the TSS nucleotide (cap1) are essential for mRNA translation, stability, and identification of the RNAs as ‘self’ by the innate immune system (2). Prokaryotic and eukaryotic RNA polymerases can also use cellular metabolites as the initiating nucleotide to generate alternatively capped RNAs (3), as shown for nicotinamide adenine dinucleotide (NAD) (4, 5).

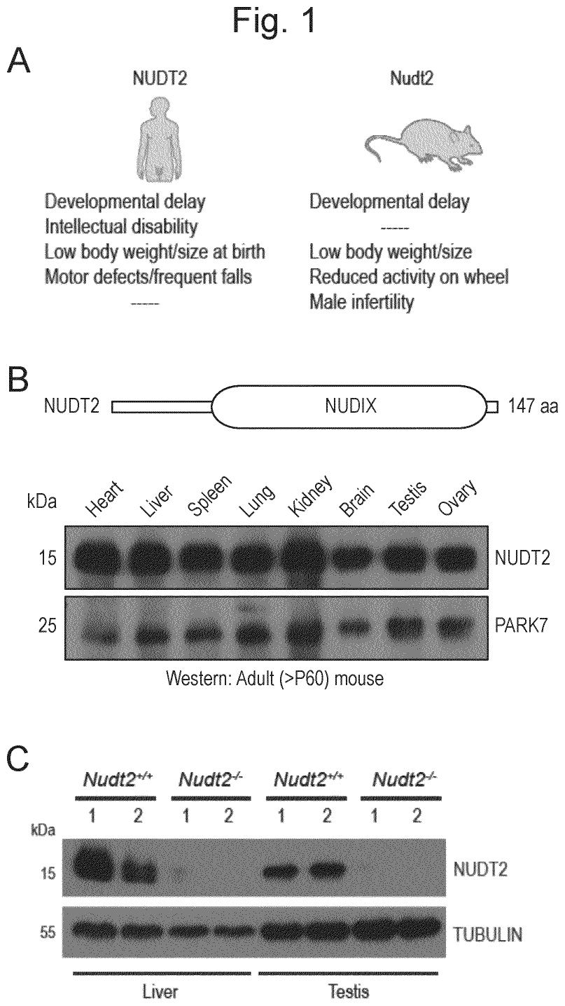

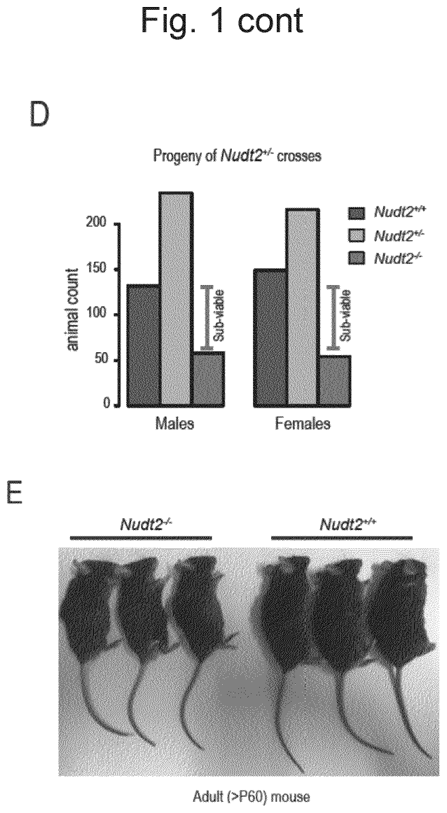

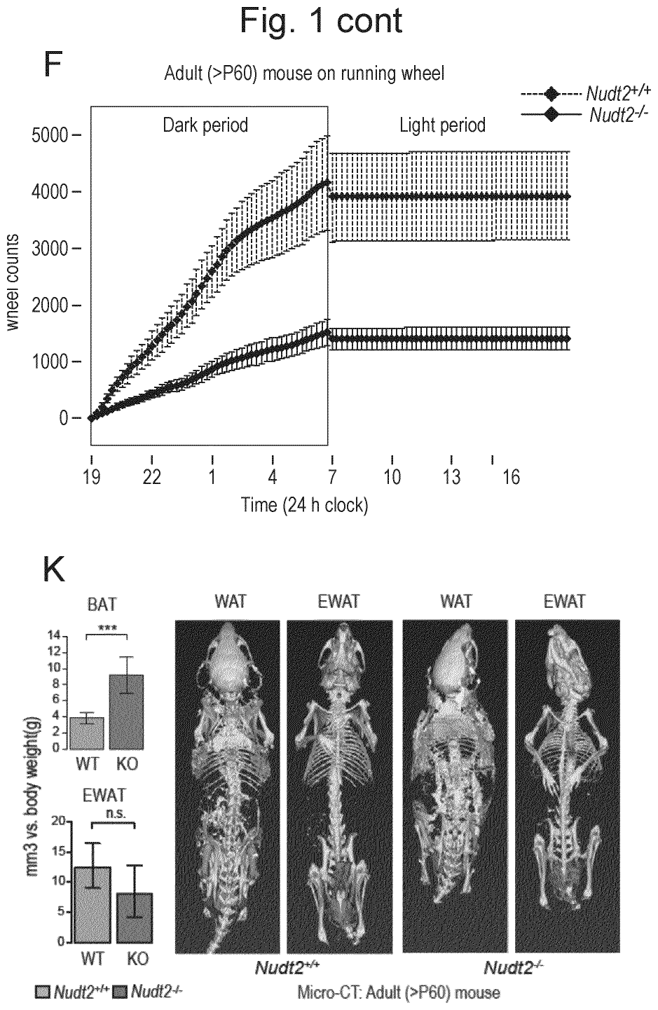

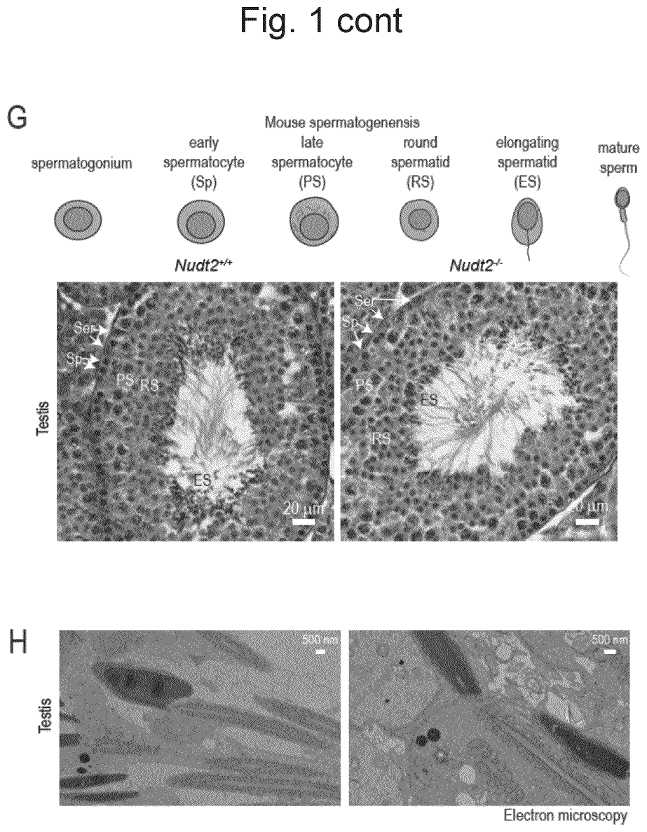

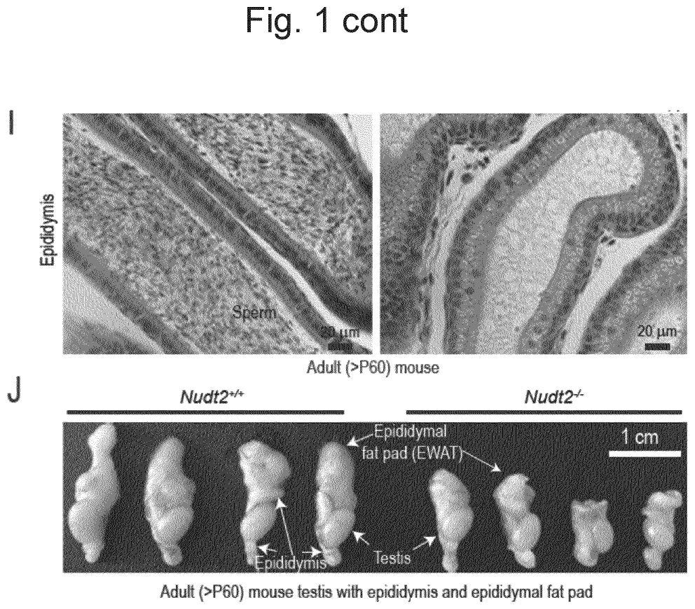

Removal of the cap by decapping enzymes leads to RNA degradation. Eight members (NUDT2, NUDT3, NUDT12, NUDT15, NUDT16, NUDT17, NUDT19, NUDT20) of the Nudix family (6) are shown to harbor m⁷G-RNA decapping activity in vitro (7). Decapping enzyme 2 (DCP2 or NUDT20) is the main m⁷G-RNA decapping activity across all eukaryotes, with Dcp2 being essential for mouse development (8). However, Nudix enzymes are primarily hydrolases acting on specific metabolite substrates (9), and based on this preference, NUDT12 was shown to be specialized for decapping NAD-RNAs (10, 11). We examined the in vivo role of NUDT2, as homozygous loss-of-function mutations in human NUDT2 causes developmental delay and intellectual disability (12, 13) (FIG. 1A), but the underlying molecular basis for this rare human disorder is not known.

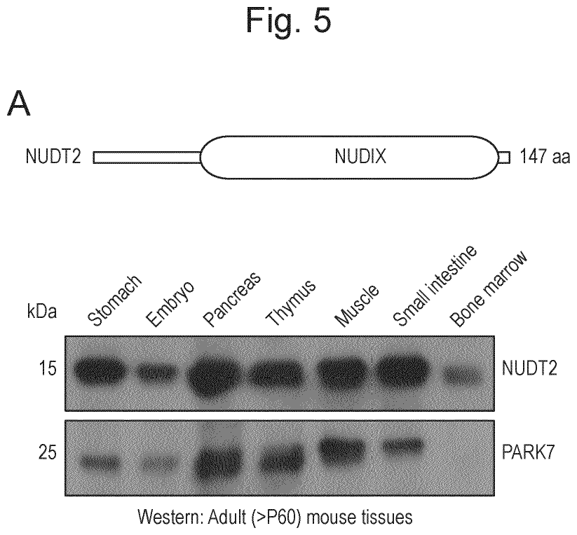

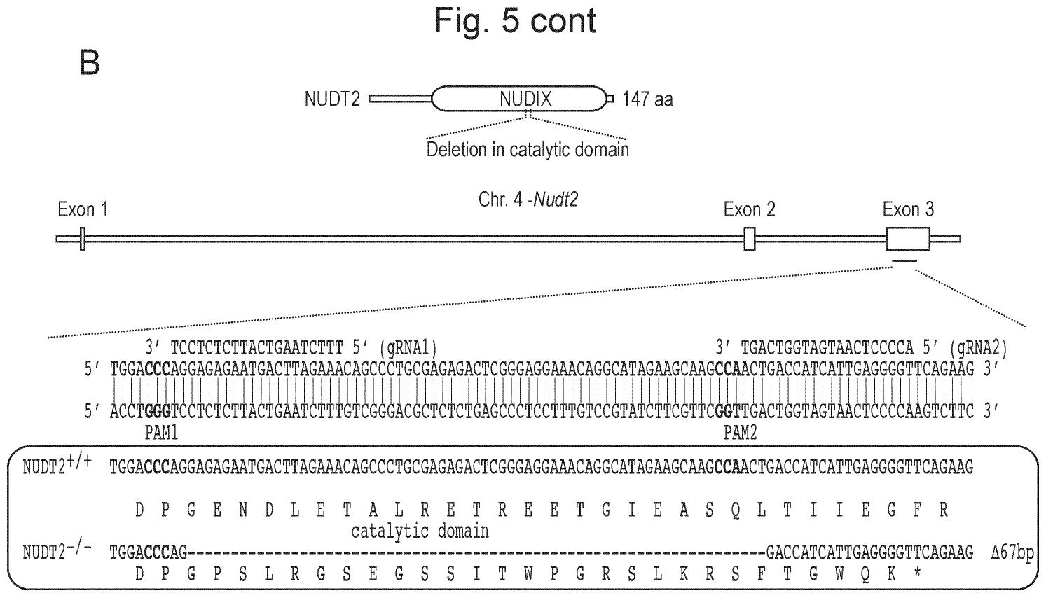

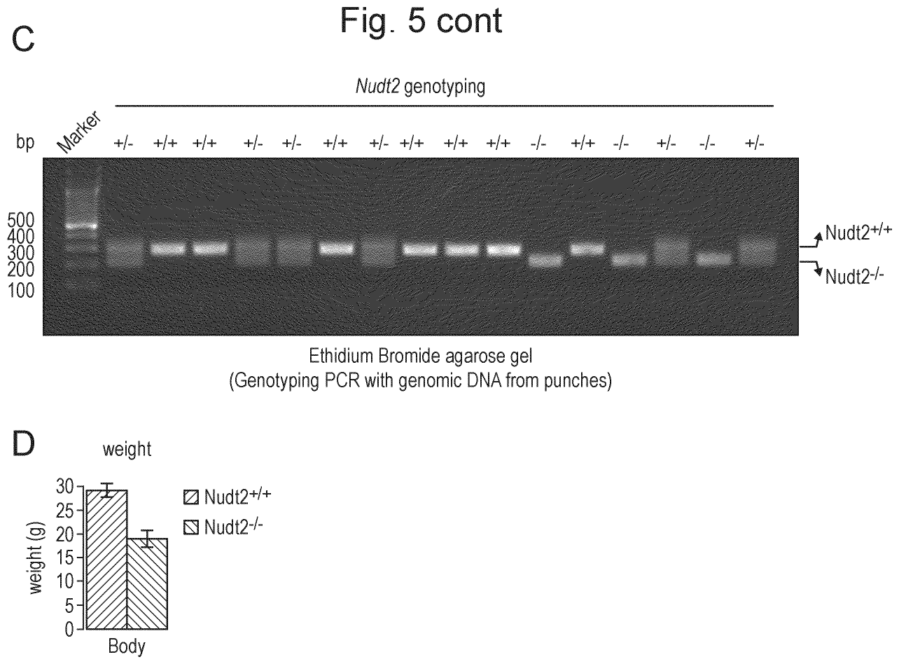

NUDT2 is a ubiquitously expressed protein (FIG. 1B and FIG. 5A). To study its physiological roles, we created a null mutant for mouse Nudt2 (FIG. 1C and FIG. 5B-C). The heterozygous (Nudt2^{+/-}) and homozygous (Nudt2^{-/-}) mutant (hereafter referred to as knockout, KO) animals are both viable, but the latter is recovered at sub-Mendelian ratios (FIG. 1D). Pointing to developmental lethality, up to 50% of the KO animals are not present at weaning age (at post-natal day 21). The most striking effect is the visibly smaller size and lower body weight of the KO animals (FIG. 1E and FIG. 5D), mirroring the developmental delay seen in human patients. Moreover, the KO animals show hugely reduced activity on a running wheel (FIG. 1F), resembling the weakness of the extremities and frequent falls noted in patients with NUDT2 mutations (12, 13).

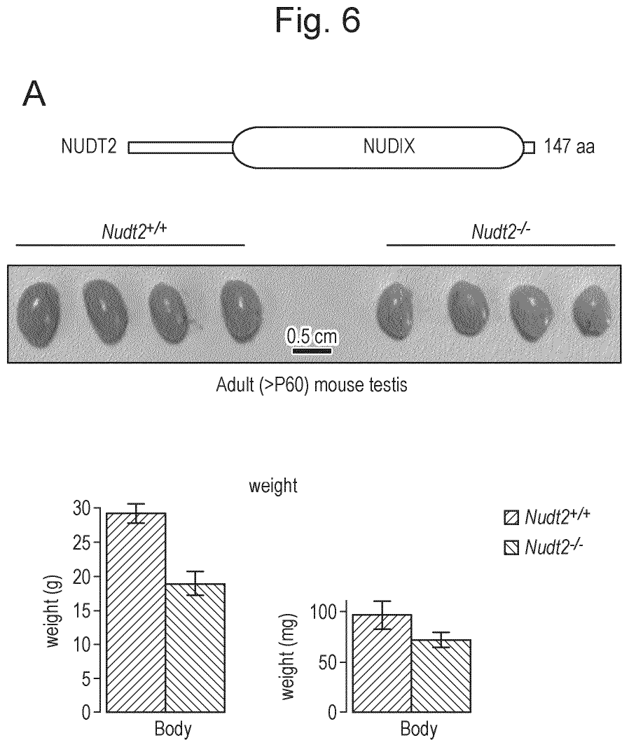

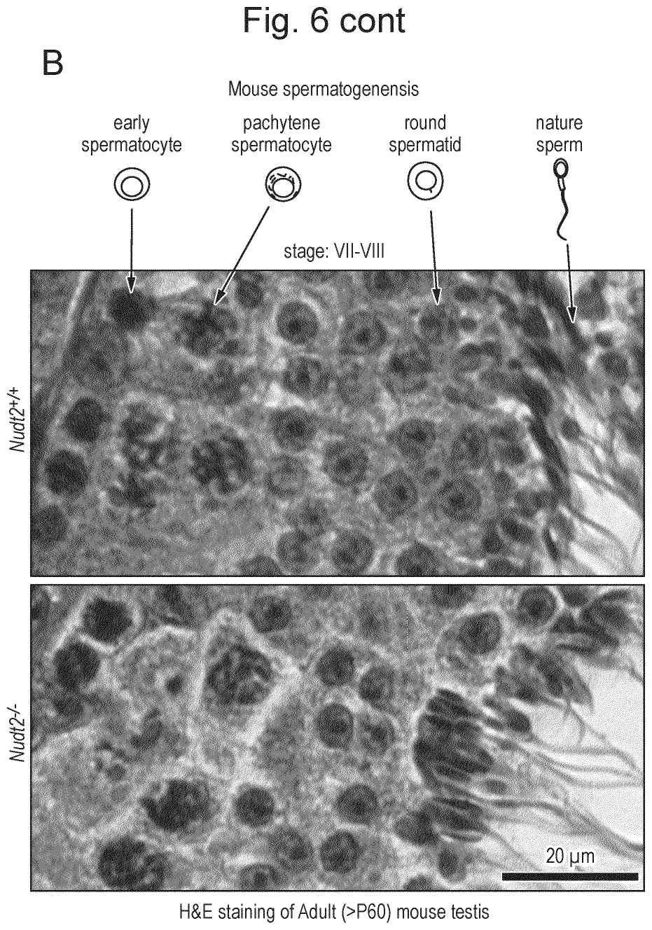

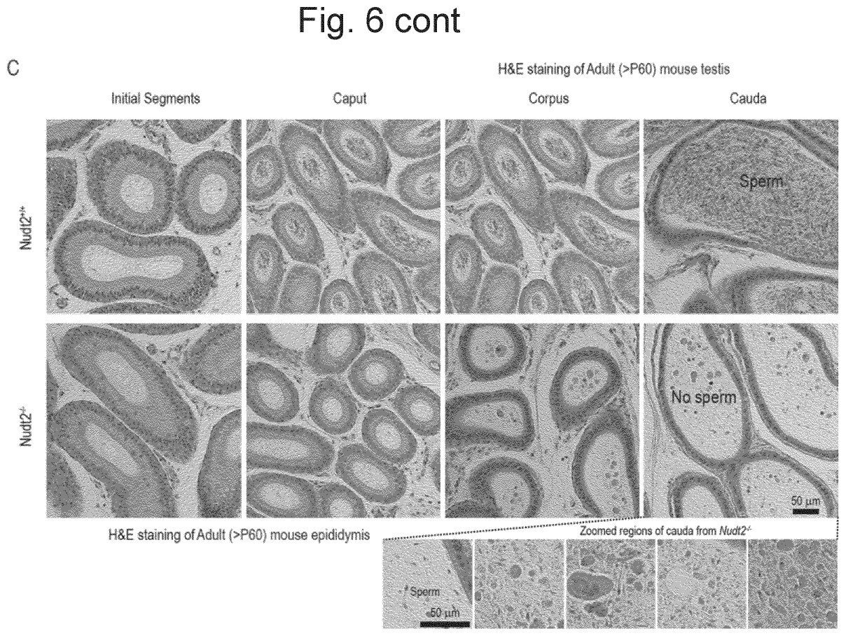

Furthermore, the Nudt2 KO male mice are sterile. The testis size and weight from the infertile males are only moderately reduced when compared to fertile control littermates (FIG. 6A). Surprisingly, histological examination shows normal spermatogenesis with a full complement of germ cells consisting of meiotic spermatocytes, post-meiotic round spermatids, and elongate spermatids populating the seminiferous tubules in the KO testes (FIG. 1G and FIG. 6B). Electron microscopy analysis confirms the presence of fully formed sperm in the mutant testes (FIG. 1H). The condensed sperm head with compacted chromatin, a tail with the normal microtubule organization, and a mid-piece with the mitochondrial sheath are all visible.

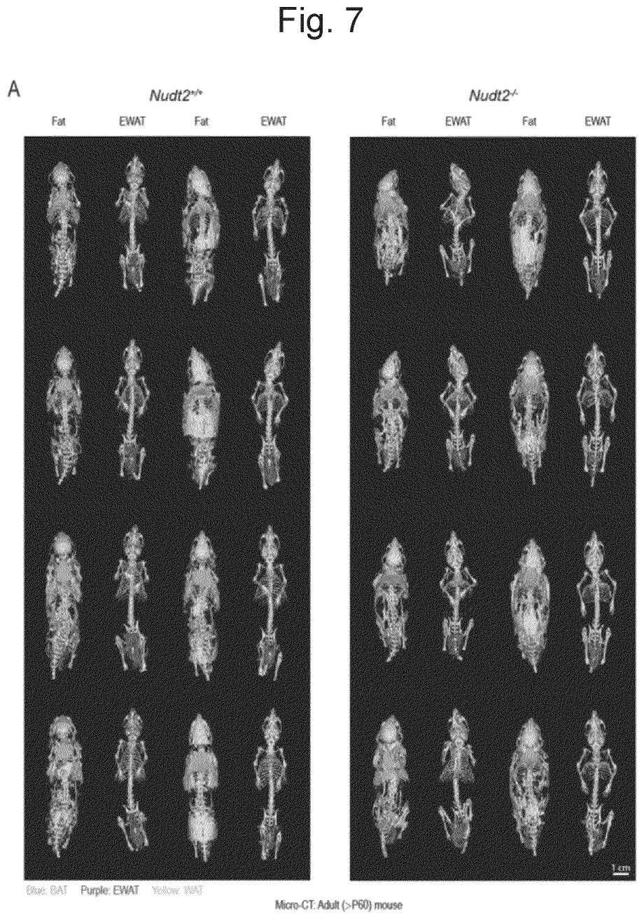

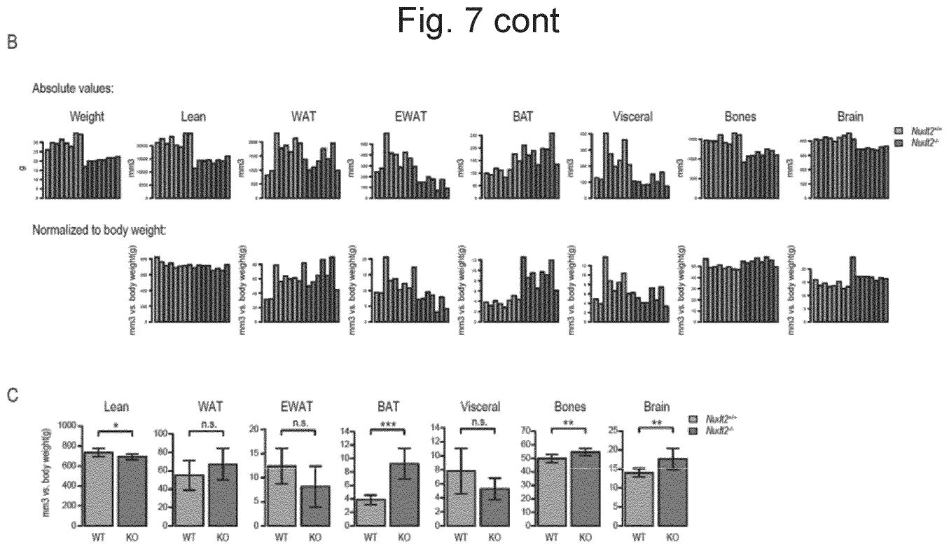

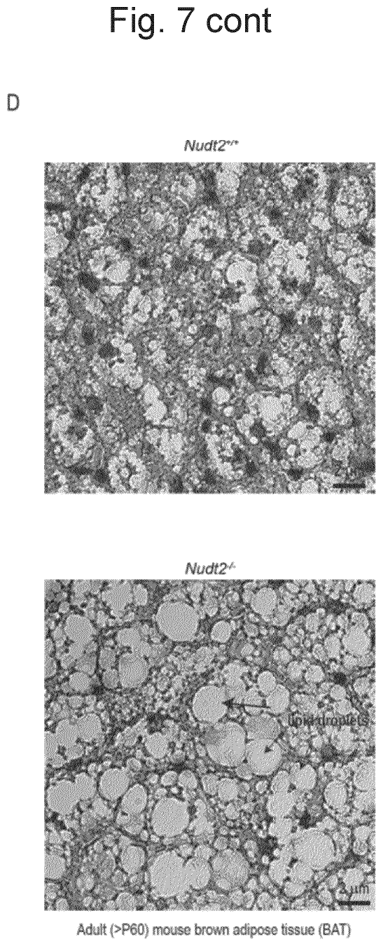

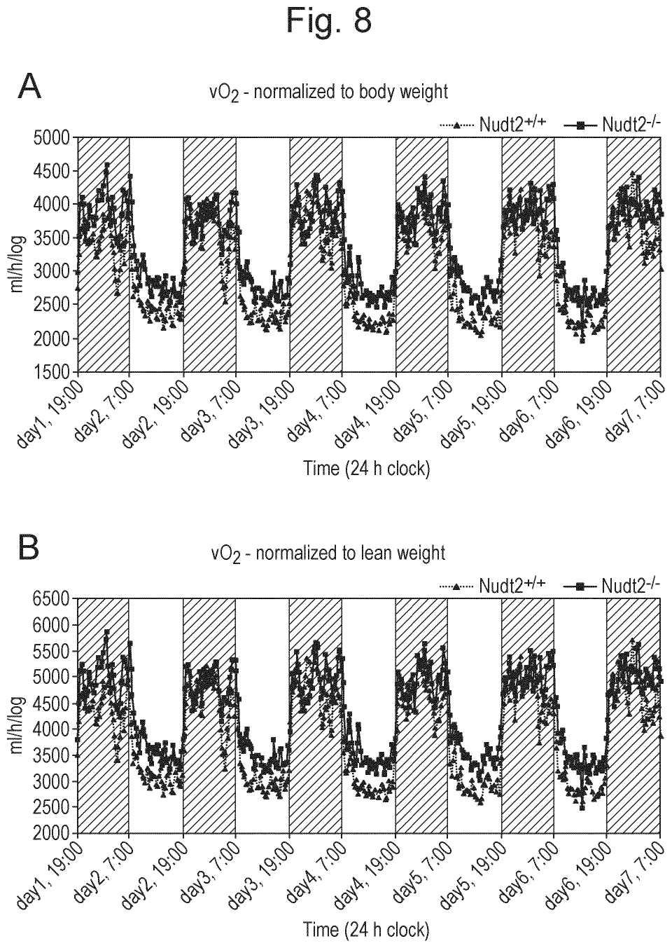

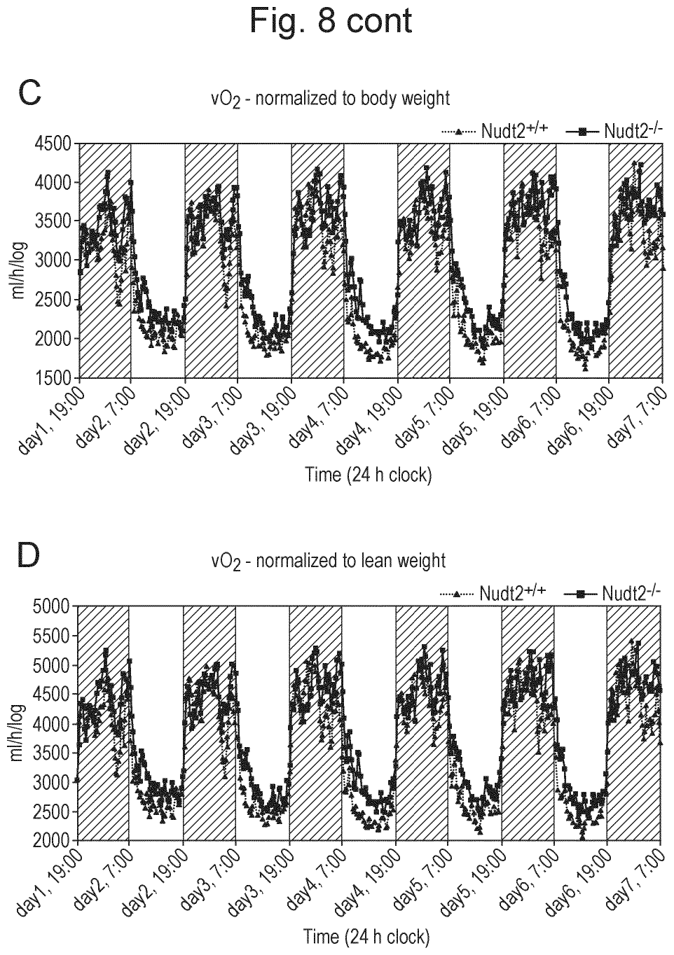

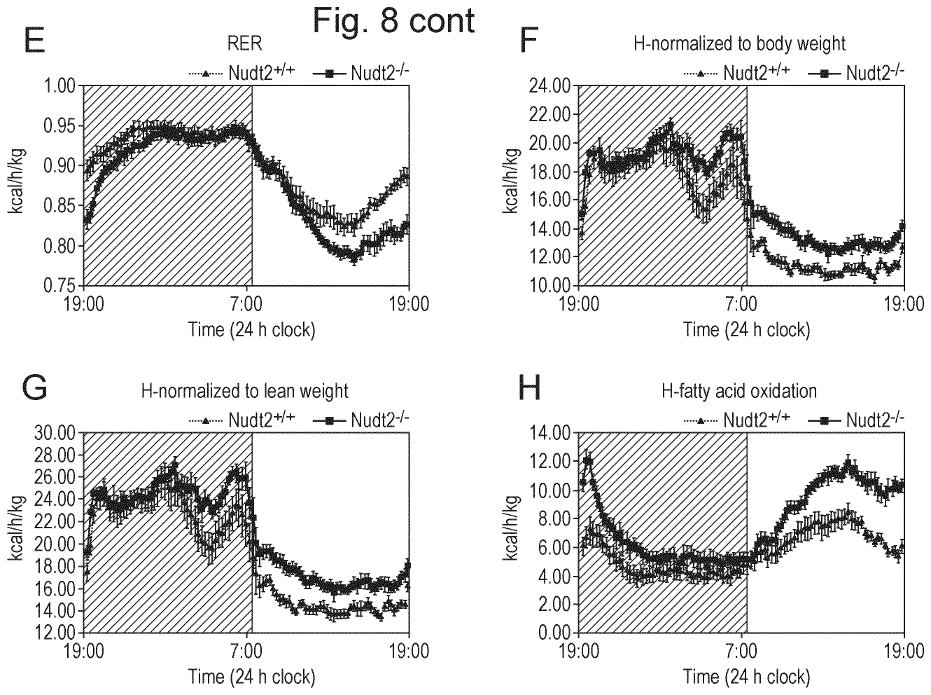

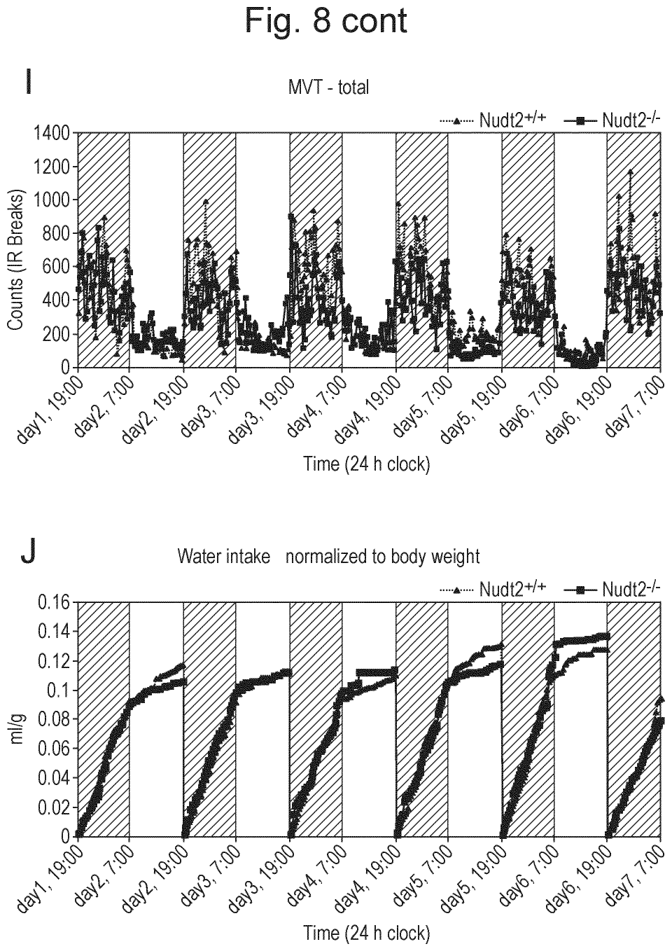

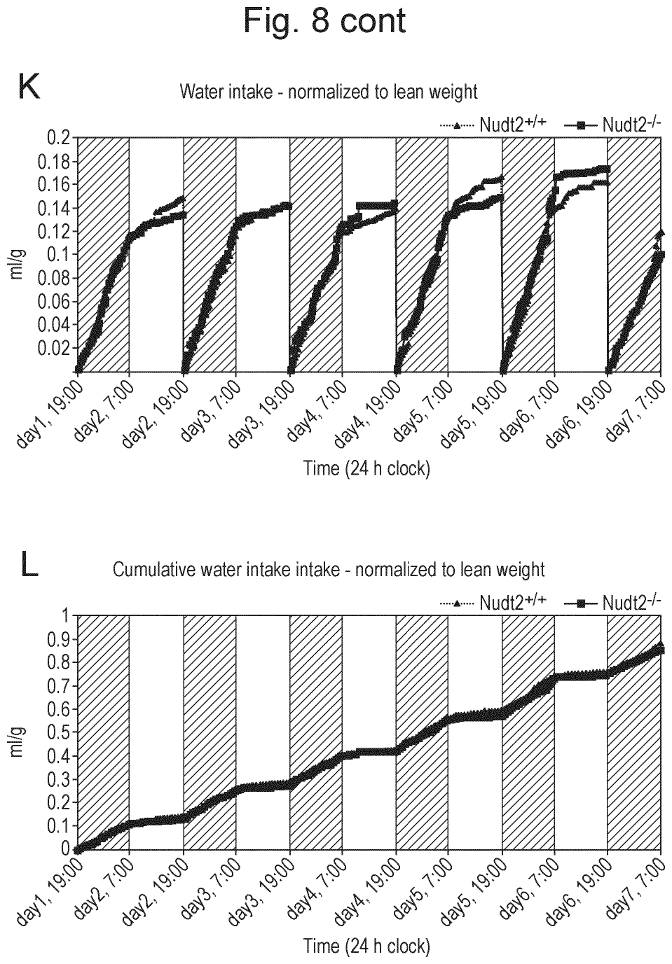

Once the sperm is produced in the testes, they gain passage into the epididymis where further maturation takes place. Consistent with the infertility of the mutant, histological examination reveals the complete absence of sperm in the epididymis (FIG. 1I and FIG. 6C). Visual examination of the epididymis shows a reduction in the epididymal white adipose tissue (EWAT) in the KO males (FIG. 1J). However, quantification using micro-computed tomography (CT) scans did not reveal any significant decrease in EWAT in the KO males (FIG. 1K). While global levels of white adipose tissue (WAT) were not altered (FIG. 7A-C), brown adipose tissue (BAT) levels are clearly increased in the KO (FIG. 1K, FIG. 7D). Measurement of calorimetric parameters indicates increased oxidation of fatty acids in the Nudt2 KO animals for energy production (FIG. 8). Epididymal fat is shown to be important for fertility (14). Thus, the analyses of the Nudt2 KO mouse reveal phenotypes shared with the NUDT2 human patients, and in addition reveal fertility and metabolic defects not reported in the patient studies.

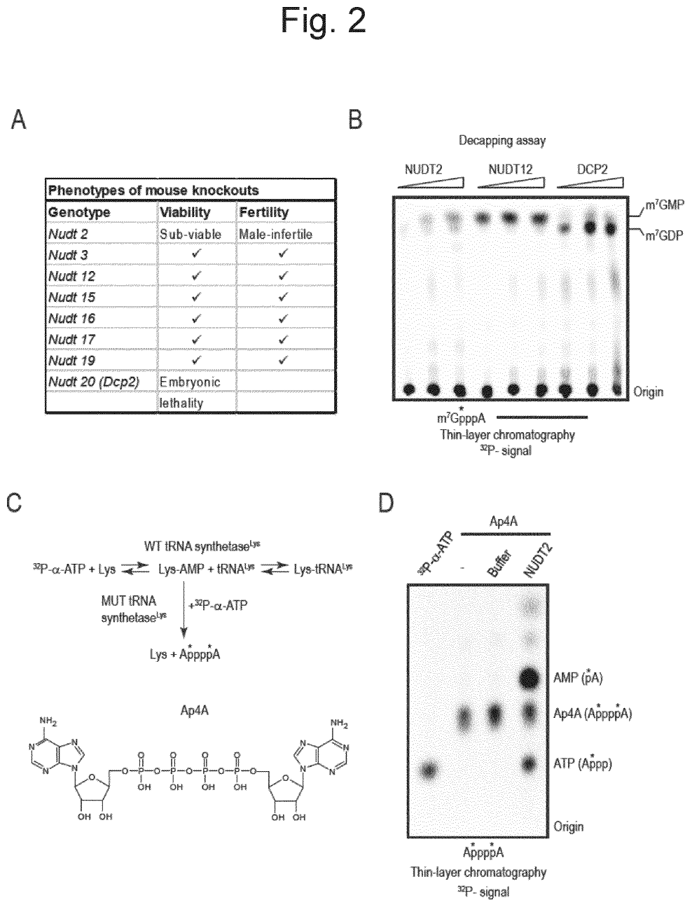

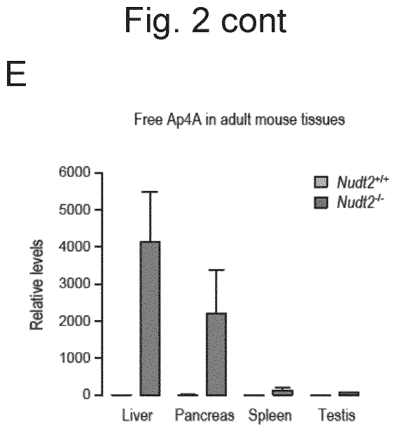

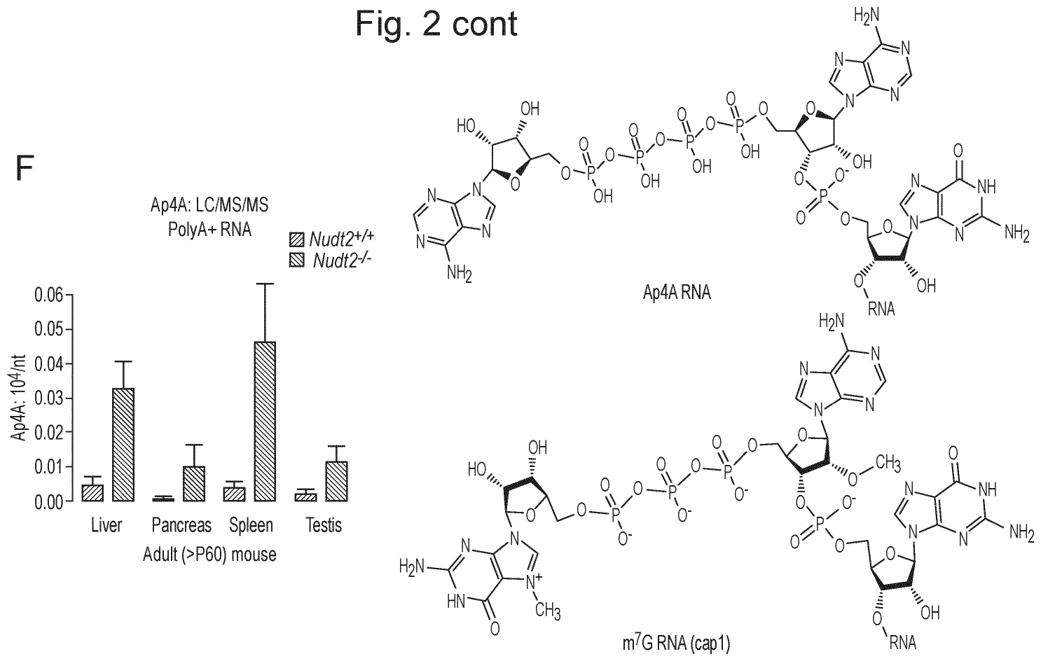

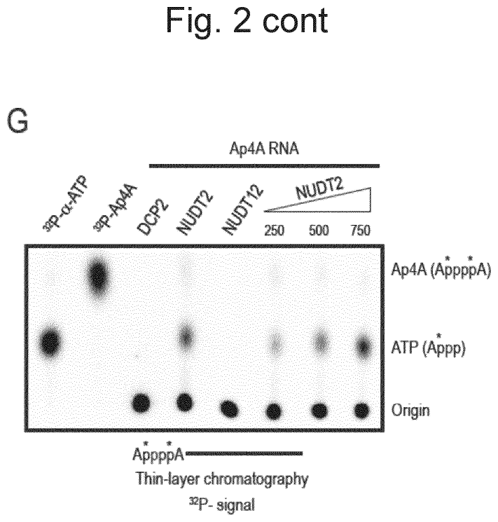

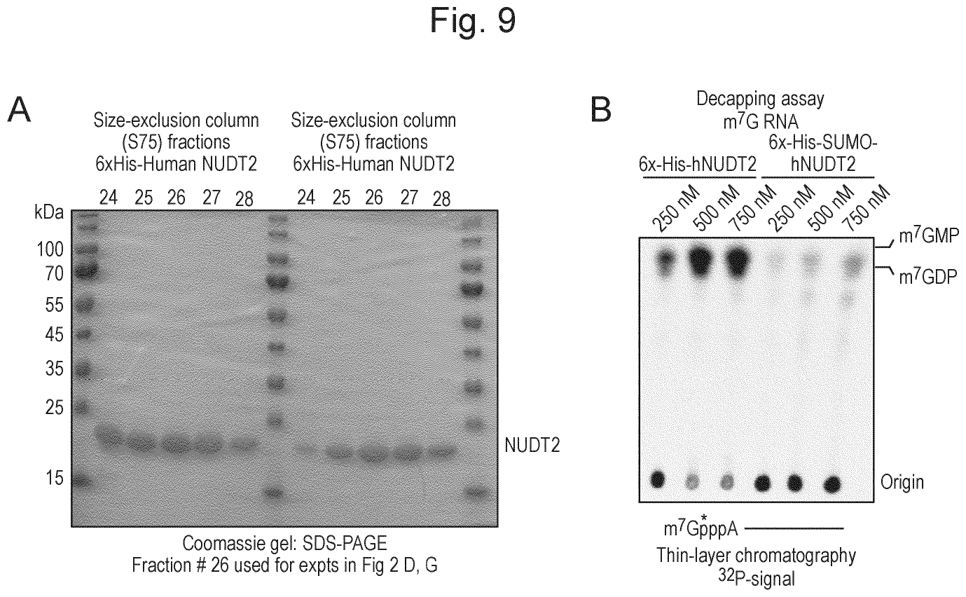

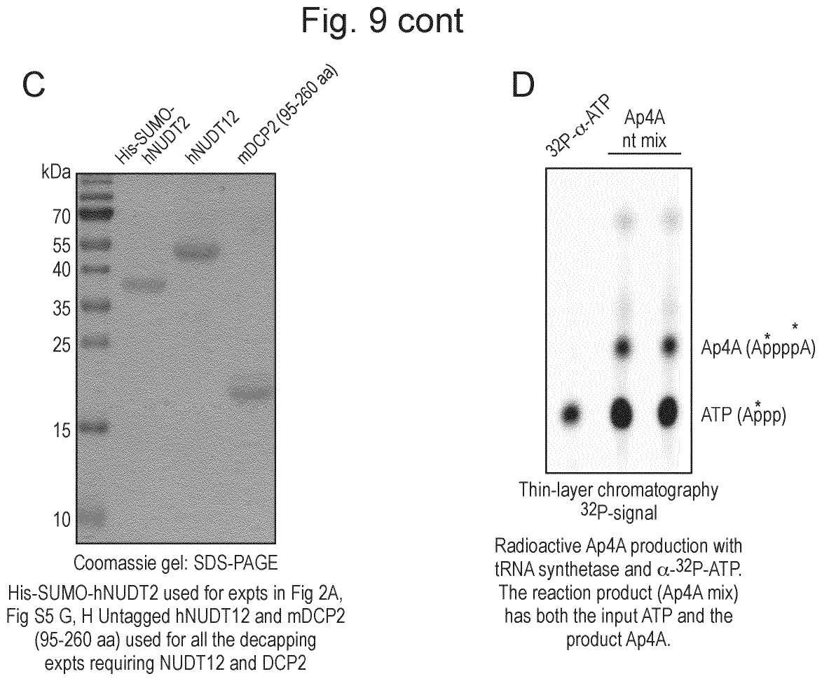

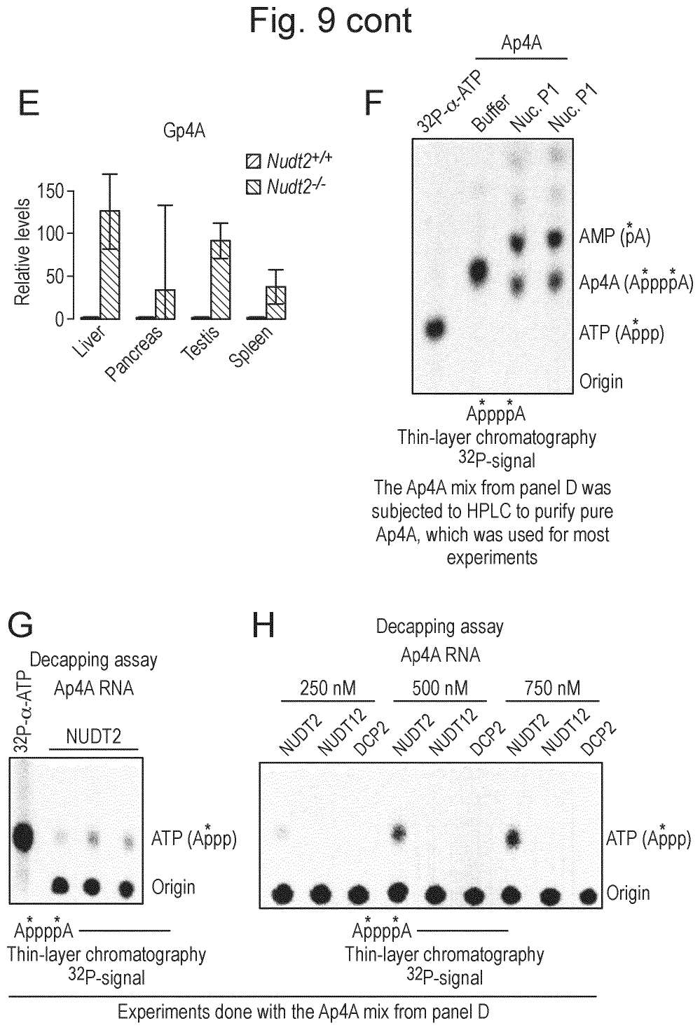

Loss of Dcp2 (Nudt20) causes embryonic lethality in mice (8), while absence of Nudt12 is tolerated (11). To test the physiological relevance of the remaining five m⁷G-RNA decapping enzymes, we created mouse knockout mutants to show that loss of Nudt3, Nudt15, Nudt16, Nudt17 and Nudt19 does not cause any obvious phenotypes in mice (FIG. 2A). Thus, we conclude that the phenotypes we observe in the Nudt2 KO animals are due to a unique in vivo activity provided by the enzyme, probably unrelated to its in vitro m⁷G-RNA decapping activity. To understand the molecular basis for these phenotypes, we focused on the biochemical activity of NUDT2. We first confirmed its m⁷G-RNA decapping activity (7) using recombinant human NUDT2 (FIG. 2B and FIG. 9A-C).

Previous studies with NUDT2 have shown that it is a hydrolase (15) for the metabolite diadenosine tetraphosphate (Ap4A), which is generated as a mis-reaction product by the lysyl tRNA synthetase (16) (FIG. 2C). We first prepared radioactive ³²P-labelled Ap4A (AppppA, p indicates radioactive 32P label) using recombinant mutant (G540Y) human lysyl tRNA synthetase that is more efficient in producing Ap4A (17) (FIG. 9D). When incubated with recombinant human NUDT2, Ap4A is mostly asymmetrically hydrolyzed into ATP (Appp) and AMP (pA) (FIG. 2D). This activity is relevant in vivo, as metabolomics analyses show that out of over 300 metabolites examined, Ap4A levels are dramatically increased in multiple Nudt2 KO mouse tissues (FIG. 2E). Another related metabolite with modestly increased levels is Ap4G (FIG. 9E). Thus, the inability to control endogenous levels of the Ap4A metabolite is the immediate biochemical consequence of loss of Nudt2.

Under conditions of stress, cellular concentrations of Ap4A and other dinucleoside polyphosphates increase in bacteria (18). Cadmium-stress in bacteria leads to capping of bacterial RNAs with Ap4A (19, 20). Such prokaryotic Ap4A-capped RNAs need to be removed by decapping enzymes for eventual degradation. Hence, we asked whether the increased levels of Ap4A in the Nudt2 KO mouse can lead to alternative capping of endogenous RNAs with Ap4A. We purified polyA+ RNA from different mutant tissues and conducted mass spectrometry. While Ap4A was detected at low levels in RNAs from wild-type tissues, the metabolite was clearly detected at higher levels (6- to 12-fold) in the RNAs from all the tested Nudt2 KO tissues (FIG. 2F). Our calculations reveal ˜6 Ap4A/106 nucleotides in Nudt2 KO polyA+ RNA. Thus, assuming an average mRNA length of 2 kb, we estimate 1 mRNA out of 83 to be capped with Ap4A. This number is an underestimation, as we find that the nuclease P1 used to hydrolyze the RNAs prior to mass spectrometry, is also able to efficiently hydrolyse Ap4A (FIG. 9F), effectively reducing the detectable Ap4A in the sample.

Nudix enzymes can act both as a hydrolase for metabolites, and as an RNA decapping enzyme when the same metabolite is incorporated into RNA 5′ ends, as shown previously for NAD and NAD-RNAs (10). To test if NUDT2 has Ap4A RNA decapping activity, we prepared cap-labelled Ap4A-RNAs (FIG. 2G and FIG. 9G-H). In vitro decapping assays with DCP2 (NUDT20), NUDT12 and NUDT2, followed by thin-layer chromatography (TLC) analyses show that only NUDT2 has Ap4A-RNA decapping activity (FIG. 2G and FIG. 9G-H). NUDT2 liberates ATP, leaving behind AMP on the RNA 5′ end. Such RNA products with a 5′ monophosphate end are ideal substrates for elimination by the cytosolic 5′-+3′ exonuclease activity of XRN1. Thus, enzymatic activity of NUDT2 should result in elimination of Ap4A-RNAs in vivo.

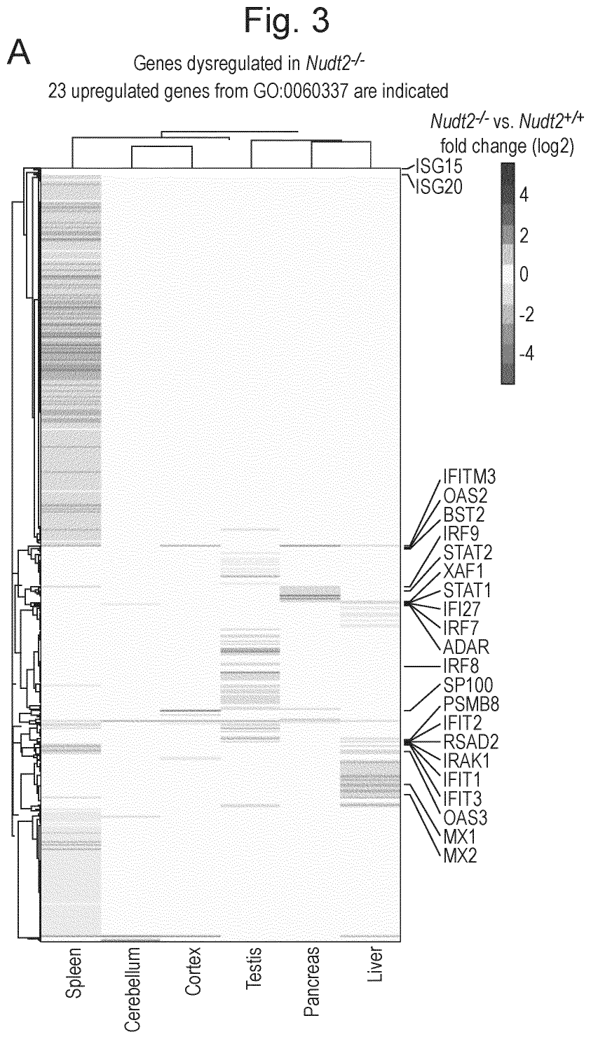

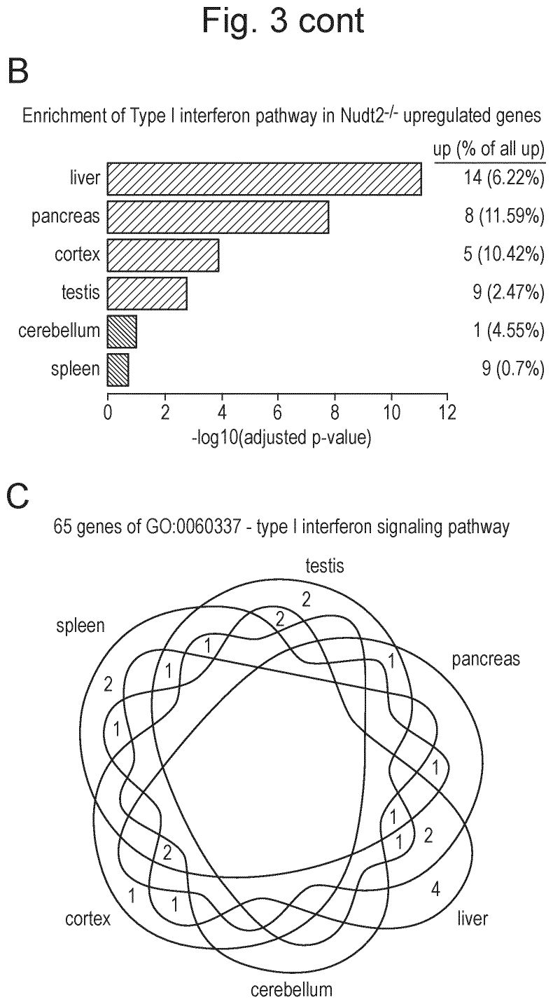

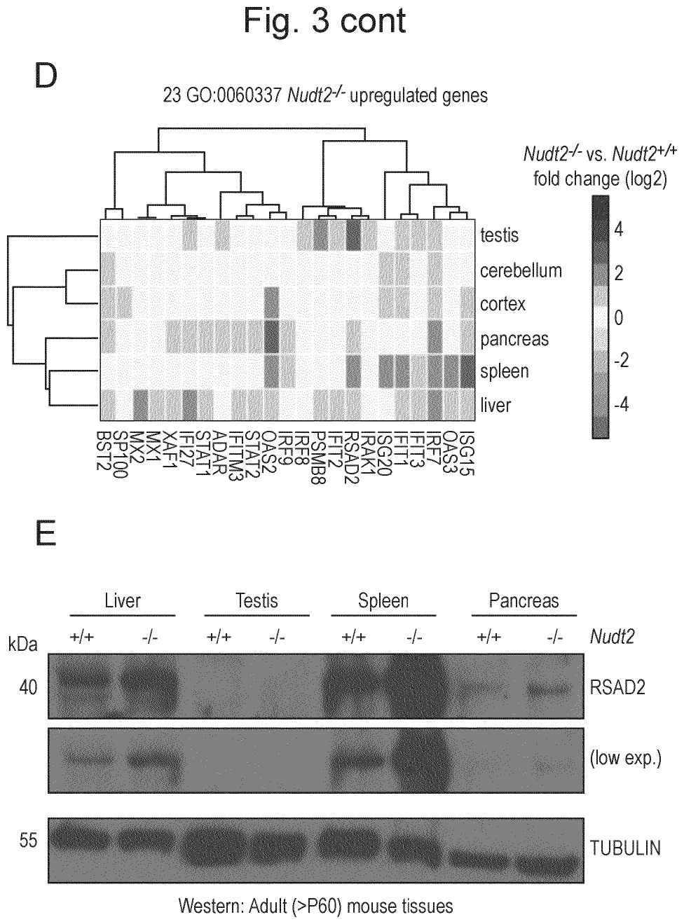

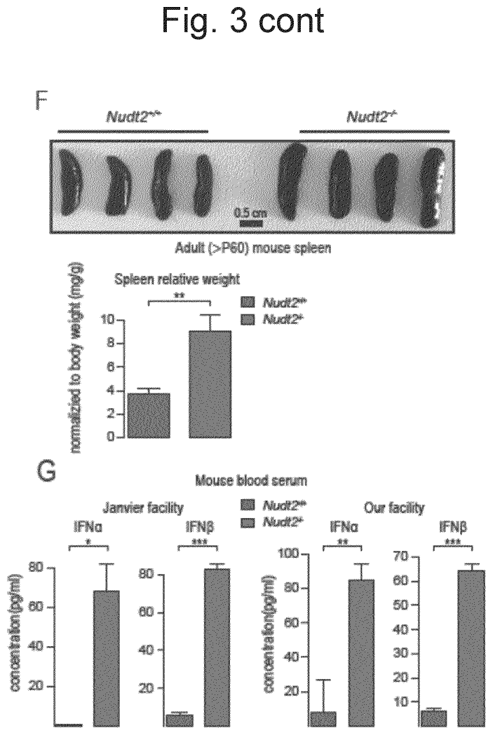

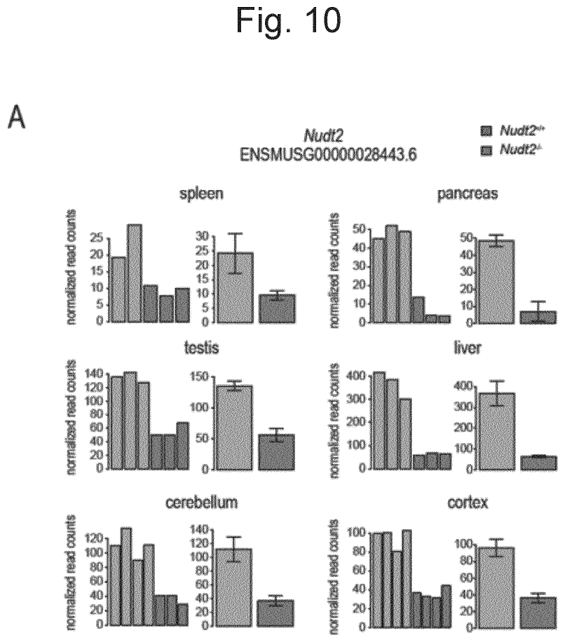

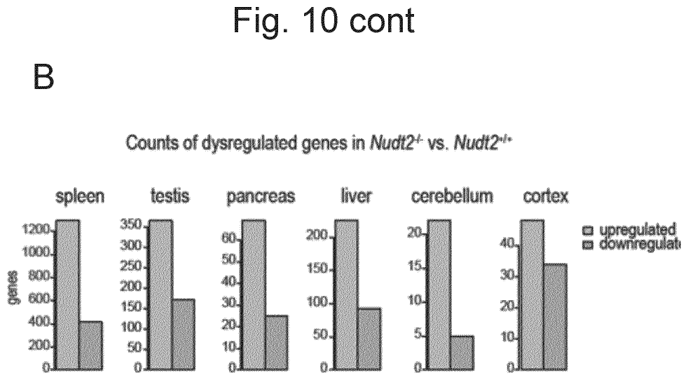

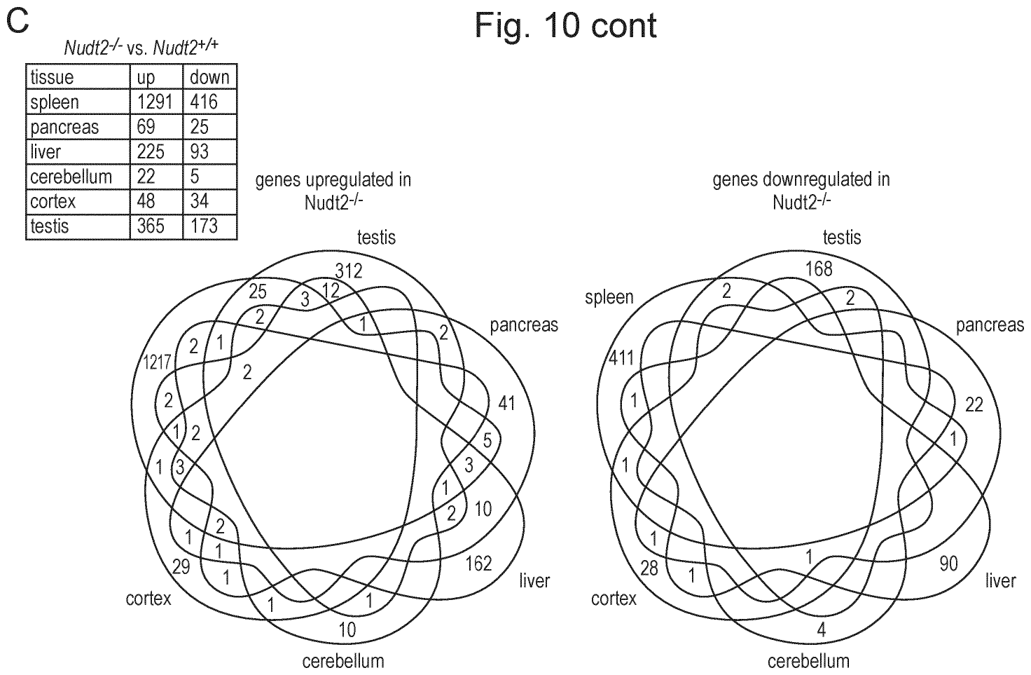

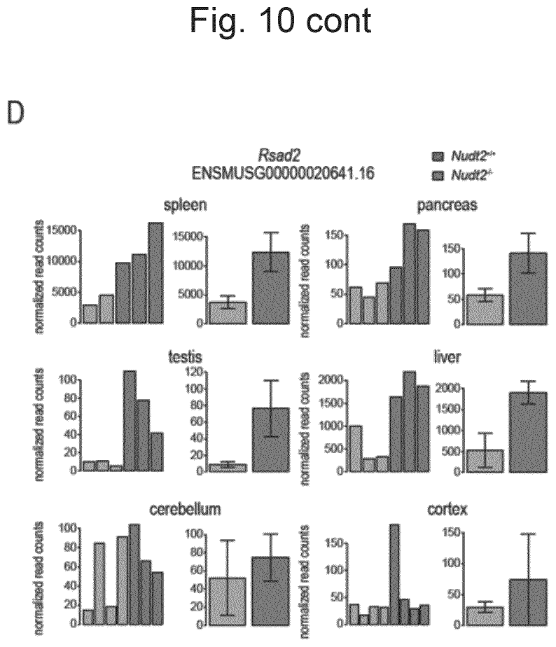

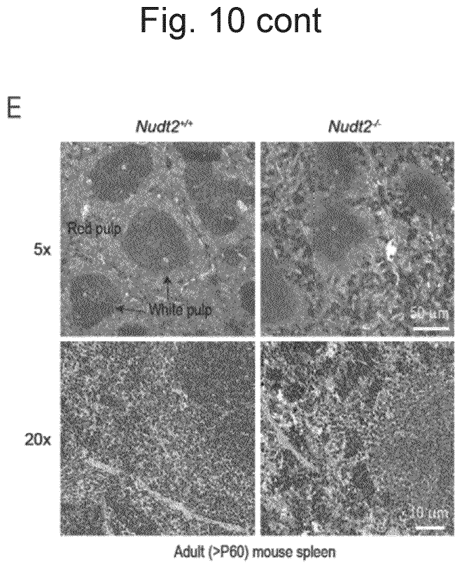

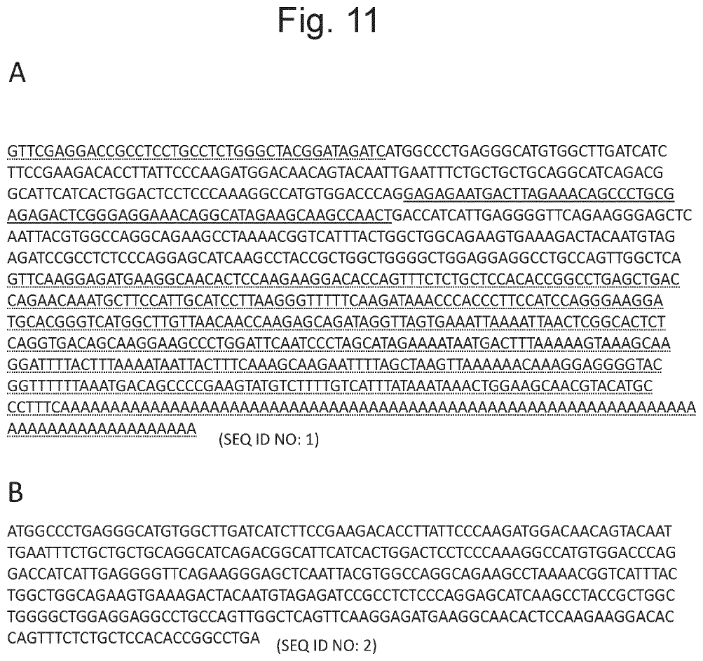

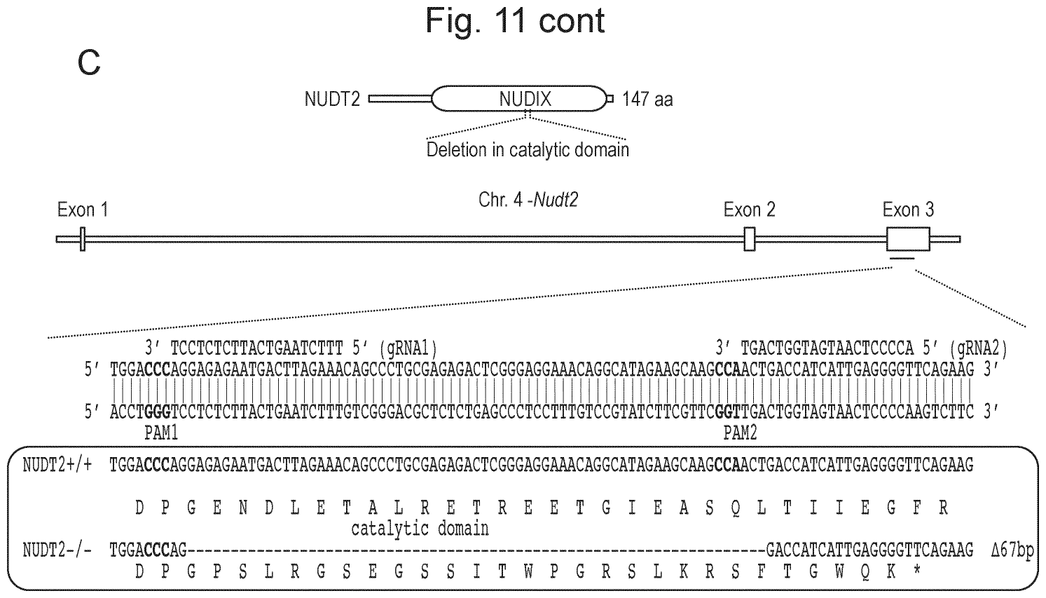

To understand the gene expression changes due to loss of Nudt2, we examined the transcriptomes in multiple KO tissues. With the exception of spleen, the overall number of genes altered in Nudt2 KO pancreas, liver, testis, and brain (cerebellum and cortex) are low (FIG. 3A and FIG. 10A-B). Although transcripts unique to each tissue are either up- or down-regulated in these datasets (FIG. 10C), one common group of genes that are upregulated in all tissues belong to the type I interferon signalling pathway (FIG. 3B-C). Interferons (IFNs) are cytokines that are secreted by pathogen-infected cells and are essential for antiviral immunity (21). In turn, the secreted IFNs trigger expression of Interferon Stimulated Genes (ISGs) in the infected and neighbouring cells to create a cell-intrinsic antiviral state (22).

In most of the Nudt2 KO tissues, we detect upregulation of the transcription factor IFN-regulatory factor 7 (IRF7) that is required for IFN expression (FIG. 3D). When IFNs bind to cell-surface receptors, a signaling pathway results in the binding of transcription factors like the signal transducer and activator of transcription 1 (STAT1), STAT2 and IRF9 to cognate DNA sequences called IFN-stimulated response element (ISRE) to drive expression of ISGs. These transcription factors are also upregulated in some of the tissues (FIG. 3D). Collectively, the different tissues express several ISGs (FIG. 3D). One broadly expressed ISG is RSAD2, with Western blotting confirming a strong induction of RSAD2 in the mutant spleen (FIG. 3E and FIG. 10D). Loss of Nudt2 has a clear impact on spleen morphology, as the organ is enlarged consistent with splenomegaly (FIG. 3F and FIG. 10E), also seen in conditions of pathogen-induced interferon stimulation in mice (23).

Type I interferons consist of the single IFN-β and multiple subtypes of IFN-α (24). Notably, although we do not detect expression of IFN mRNAs in the tested tissues, direct detection and quantification using ELISA shows a ˜6 to 8-fold increase in levels of IFN-α and IFN-β in blood serum from the Nudt2 KO animals (FIG. 3G). Importantly, the elevated interferon levels in blood serum, small body size and splenomeghaly in KO animals, were all re-confirmed in mice re-derived and bred at a commercial animal facility (FIG. 3G). Taken together, we identify a general activation of the type I IFN-dependent gene expression pathway as the common gene expression consequence in the Nudt2 KO mouse tissues.

Interferon production is triggered after sensing of foreign agents called pathogen-associated molecular patterns (PAMPs) via sensor proteins collectively called pattern recognition receptors (PPRs). Double-stranded RNAs, RNAs with a triphosphophate 5′ end, m⁷G cap0 structure, or cytoplasmic methylated DNA are all considered ‘non-self’ by the innate immune system (25). Sensing the status of the RNA 5′ end is part of this innate defense mechanism. Indeed, viruses that replicate in the cytoplasm encode their own enzymes to generate the m′G cap1 structure, to pass off as ‘self’ RNAs (2, 26). Given that Ap4A RNA caps are chemically different from that of the endogenous ‘self’ m⁷G cap1 structure, we wondered whether the increased levels of the endogenous Ap4A RNA in the Nudt2 mutant might be the trigger for the observed IFN-dependent gene expression.

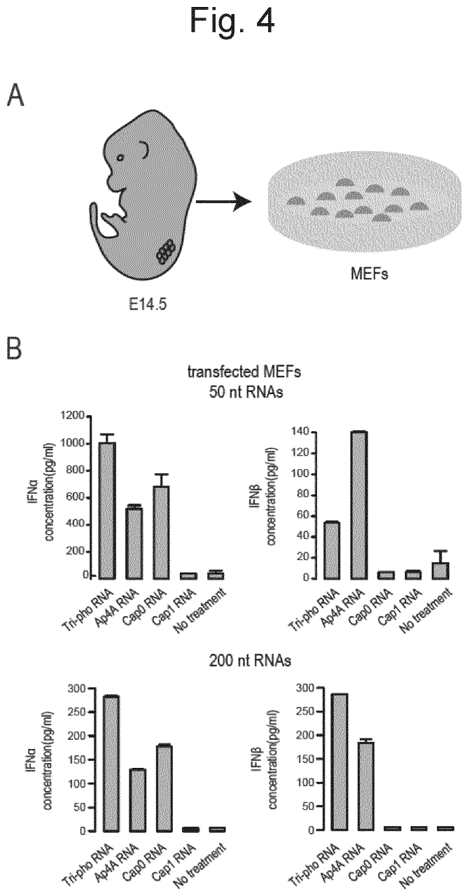

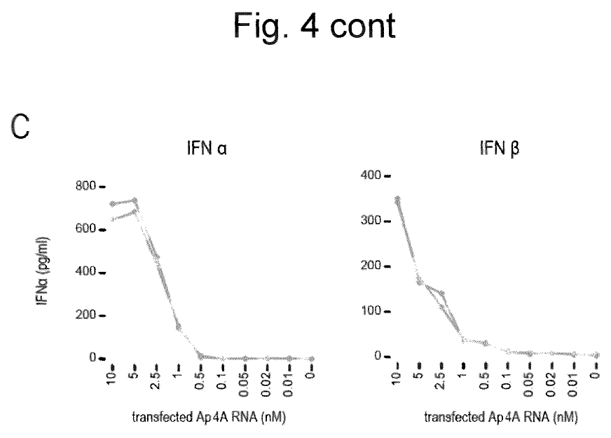

To this end, we transfected mouse embryonic fibroblasts (MEFs) with single-stranded RNAs having different 5′ ends and quantified IFN production in the cell culture media 24 h-post transfection (FIG. 4A). ELISA measurements show the expected induction of both IFN-α and IFN-β with the RNA having a triphosphorylated 5′ end (tri-pho RNA), while the RNA with a cap0 structure (cap0 RNA) triggers IFN-α production (FIG. 4B). Strikingly, Ap4A RNAs elicited a strong induction of both IFN-α and IFN-β (FIG. 4B). The effect was not dependent on the length (either 200 nt or 50 nt) of the transfected RNA. As expected, the ‘self’ RNA with m⁷G cap1 structure (cap1 RNA) did not lead to IFN production and behaved similar to mock-transfected cells (FIG. 4B). Transfection experiments with a range (0.01 to 10 nM) of Ap4A RNA concentrations lead us to conclude that a certain threshold level needs to be crossed for efficiently triggering IFN production (FIG. 4C). These results show that Ap4A RNA is sensed as ‘non-self’ by the innate immune system in mammalian cells.

We propose a model where increased Ap4A metabolite levels and accumulation of Ap4A RNA in the Nudt2 mutant (FIG. 2) results in induction of the IFN-dependent gene expression pathway in all mutant tissues (FIG. 3). We were not able to identify the interferon-producing cell types, as the tested Nudt2 KO tissues and KO MEFs did not express IFN mRNAs. Similarly, RNA-seq analysis of human NUDT2 KO cell lines also did not report any IFN expression (27). We propose that specific cell types initially produce the IFN, which is then spread via the circulatory system to initiate ISG expression in the various tissues. In this context, the expression of ISGs in the cerebral cortex, which is behind the blood-brain barrier, supports the existence of an intrinsic trigger. Type I IFN-dependent gene expression is shown to result in reduced brain function in aged brains (28), but the phenotypic consequences in the Nudt2 mutant could be due to chronic activation of the IFN pathway in the whole body. We note that sustained IFN activation by PAMPs is shown to cause hypothermia and weight loss and morbidity (29), features also found in the Nudt2 KO mice, likely explaining the shift to increased levels of thermogenic BAT (FIG. 1K).

We do not rule out a contribution from the free Ap4A metabolite itself to the observed phenotypes. Endogenous levels of Ap4A briefly increase under physiological conditions in mast cells undergoing IgE-antigen-mediated stimulation (15). Under these conditions, free Ap4A acts as a second messenger, and is bound by HINT1 to remodel a complex, releasing the transcription factor MITF (30) to drive expression of specific genes. However, loss of Hlint1 does not affect mouse fetal or adult development (31), pointing to additional reasons for the severe phenotypes seen in the Nudt2 mutant. Increased Ap4A levels is also shown to promote directionality and mobility of dendritic cells (32). We propose that in wildtype mouse tissues, action of NUDT2 keeps the levels of Ap4A and endogenous Ap4A RNAs in check, preventing the unintended IFN induction in the absence of an invading pathogen. Although we do not know the status of IFN pathway in human patients with NUDT2 mutation, our study suggests that inhibition of the IFN pathway is likely to be a viable path for therapeutic intervention in the patients.

### Materials and Methods

### Animal Work

Mutant mice were generated at the Transgenic Mouse Facility of University of Geneva. The Nudt2 mice were bred in the Animal Facility of Sciences III, University of Geneva. The animals were kept in ventilated cages and regular tests for pathogens conducted. The wildtype and knockout littermates were kept in the same cage. To reconfirm the findings at a different facility, the Nudt2 animals were re-derived from frozen sperm in the Specific Pathogen Free (SPF) facility of Janvier Labs, France. Mice were bred and raised at the Janvier Labs facilities, then delivered to our labs at University of Geneva. Such mice were allowed to rest overnight in a laminar flow hood (outside our animal facility) and sacrificed for experiments the next day. The phenotypes of small body size, splenomeghaly and elevated interferon levels were confirmed in such mice too.

### Nudt2 Knockout Mice

The Nudt2 gene locus is located on mouse chromosome 4 and consists of 3 exons (FIG. 5B) and encodes for a protein with 147 aa (NCBI: NP_079815). We targeted the Nudt2 locus in mouse embryos of the B6D2F1/J hybrid line (also called B6D2; The Jackson Laboratory, stock no. 100006). It is a cross between C57BL/6J (B6) and DBA/2J (D2), and heterozygous for all B6 and D2 alleles. Single-cell mouse embryos were injected with two different guide RNAs (gRNAs) that direct the DNA endonuclease Cas9 to delete a region within exon 3 (FIG. 5B). Founder mice were identified by genotyping PCR (FIG. 5C) and crossed with wildtype C57BL/6J (Janvier) partners to obtain germline transmission. We identified a mouse line with 67 bp deletion in the exon 3 that removes the critical hydrolytic residues (Nudix box) within the Nudix hydrolase domain. This deletion results in frameshift leading to creation of a premature termination codon. This ensures complete removal of the mutant transcript via nonsense-mediated decay (NMD). Indeed, Western analysis confirms the homozygous Nudt2 mutant mice to be complete nulls as they lack the NUDT2 protein (FIG. 1C).

Heterozygous Nudt2^{+/-} mice of both sexes were viable, while homozygous Nudt2^{-/-} mice displayed preweaning ˜50% lethality (FIG. 1D). Heterozygous Nudt2^{+/-} animals were fertile when crossed with each other or with wildtype partners. However, homozygous Nudt2^{-/-} males were infertile when crossed with partners of any genotype. Interestingly, although homozygous Nudt2^{-/-} females were fertile when crossed with wildtype partners, they did not produce offspring when crossed with heterozygous Nudt2^{+/-} males. Homozygous Nudt2^{-/-} mice of both sexes were smaller in size (FIG. 1E), with reduced body weight (FIG. 5D), and had a splenomegaly phenotype (FIG. 3G). RNA-seq analyses (Table 1) revealed stimulation of type 1 interferon under normal husbandry conditions (FIG. 3).

TABLE S1
All deep sequencing datasets generated in this study.
Related to STAR Methods.

| sample | description | reads |
|---|---|---|
| WHR45 | NUDT2_spleen_WT_1 | 23598888 |
| WHR46 | NUDT2_spleen_WT_2 | 39186451 |
| WHR42 | NUDT2_spleen_KO_1 | 57006079 |
| WHR43 | NUDT2_spleen_KO_2 | 58674880 |
| WHR44 | NUDT2_spleen_KO_3 | 19416195 |
| WHR51 | NUDT2_testis_WT_1 | 39586573 |
| WHR52 | NUDT2_testis_WT_2 | 41855203 |
| WHR53 | NUDT2_testis_WT_3 | 45549620 |
| WHR48 | NUDT2_testis_KO_1 | 41983788 |
| WHR49 | NUDT2_testis_KO_2 | 42666656 |
| WHR50 | NUDT2_testis_KO_3 | 41462721 |
| WHR150 | NUDT2_pancreas_WT_1 | 75099482 |
| WHR151 | NUDT2_pancreas_WT_2 | 56530815 |
| WHR152 | NUDT2_pancreas_WT_3 | 54022530 |
| WHR153 | NUDT2_pancreas_KO_1 | 91187303 |
| WHR154 | NUDT2_pancreas_KO_2 | 56706189 |
| WHR155 | NUDT2_pancreas_KO_3 | 62585930 |
| WHR165 | NUDT2_liver_WT_1 | 61454599 |
| WHR166 | NUDT2_liver_WT_2 | 51854553 |
| WHR167 | NUDT2_liver_WT_3 | 77208008 |
| WHR168 | NUDT2_liver_KO_1 | 71103744 |
| WHR169 | NUDT2_liver_KO_2 | 67020019 |
| WHR170 | NUDT2_liver_KO_3 | 62966945 |
| WHR249 | NUDT2_cerebellum_WT_1 | 43466609 |
| WHR250 | NUDT2_cerebellum_WT_2 | 52222122 |
| WHR251 | NUDT2_cerebellum_WT_3 | 37372697 |
| WHR252 | NUDT2_cerebellum_WT_4 | 43552034 |
| WHR253 | NUDT2_cerebellum_KO_1 | 67137324 |
| WHR254 | NUDT2_cerebellum_KO_2 | 72288040 |
| WHR255 | NUDT2_cerebellum_KO_3 | 28532219 |
| WHR257 | NUDT2_cortex_WT_1 | 33318405 |
| WHR258 | NUDT2_cortex_WT_2 | 67992369 |
| WHR259 | NUDT2_cortex_WT_3 | 39328862 |
| WHR260 | NUDT2_cortex_WT_4 | 36335166 |
| WHR261 | NUDT2_cortex_KO_1 | 35395168 |
| WHR262 | NUDT2_cortex_KO_2 | 39451440 |
| WHR263 | NUDT2_cortex_KO_3 | 44223573 |
| WHR264 | NUDT2_cortex_KO_4 | 32483737 |

Preparation of gRNAs: A cloning-free method was used to prepare DNA template for in vitro transcription of the chimeric crRNA-tracrRNA, termed single guide RNA (sgRNA or gRNA). In brief, a common reverse primer (CRISPR sgR primer) and a gene-specific forward primer (CRISPR F primer) with T7 promoter sequence was used to PCR amplify the single-stranded sgDNA template. Primer sequences are provided in Table 2.

TABLE 2
DNA and RNA oligonucleotides used in this study

| Name | Sequence | Comments |
|---|---|---|
| NUDT2 Mouse | | |
| generation | | |
| WH-Oligo30 | GAAATTAATACGACT | CRISPR F primer-1 targeting |
| (SEQ ID NO: 10) | CACTATAGGTTTCTA | mNUDT2 (gene specific |
| | AGTCATTCTCTCCTG | sequence underlined) |
| | TTTTAGAGCTAGAAA | |
| | TAGC | |
| WH-Oligo31 | GAAATTAATACGACT | CRISPR F primer-2 targeting |
| (SEQ ID NO: 11) | CACTATAGGACCCCT | mNUDT2(gene specific |
| | CAATGATGGTCAGTG | sequence underlined) |
| | TTTTAGAGCTAGAAA | |
| | TAGC | |
| WH-Oligo60 | AAAAGCACCGACTCG | Common reverse primer for |
| (SEQ ID NO: 12) | GTGCCACTTTTTCAA | preparing template for guide |
| | GTTGATAACGGACTA | RNA |
| | GCCTTATTTTAACTT | |
| | GCTATTTCTAGCTCT | |
| | AAAAC | |
| WH-Oligo98 | TATTAACCATCTCCA | Forward primer for NUDT2 KO |
| (SEQ ID NO: 13) | GAGGAAAG | mouse genotyping |
| WH-Oligo100 | ACTTCTGCCAGCCAG | Reverse primer for NUDT2 KO |
| (SEQ ID NO: 14) | TAAAT | mouse genotyping |
| In vitro | | |
| transcription of | | |
| capped RNA | | |
| WH-Oligo-421 | CAGTAATACGACTCA | DNA oligo for in vitro |
| (SEQ ID NO: 15) | CTATTAGTCCGTCCG | transcription of 50 nt RNAs, |
| | CCGCCCTTGCTGGTC | sense |
| | TTTCTCGCCCGCTGC | |
| | TGCCTGCCTG | |
| WH-Oligo-424 | CAGGCAGGCAGCAGC | DNA oligo for in vitro |
| (SEQ ID NO: 16) | GGGCGAGAAAGACCA | transcription of 50 nt RNAs, |
| | GCAAGGGCGGCGGAC | antisense |
| | GGACTAATAGTGAGT | |
| | CGTATTACTG | |
| WH-Oligo-297 | CAGTAATACGACTCA | Forward primer for amplify |
| (SEQ ID NO: 17) | CTATTAGTCCG | dsDNA template, which is |
| | | used for in vitro transcription |
| | | of 200 nt RNAs |
| WH-Oligo-298 | AAACCCACCAAAGGC | Reverse primer for amplify |
| (SEQ ID NO: 18) | AGAC | dsDNA template, which is |
| | | used for in vitro transcription |
| | | of 200 nt RNAs |
| Constructs | | |
| WH-Oligo48 | GCtctagaATGGCCT | Forward primer for cloning |
| (SEQ ID NO: 19) | TGAGAGCATGTG | hNUDT2 into pCI-neo |
| | | plasmid |
| WH-Oligo49 | ATTTgcggccgcTCA | Reverse primer for cloning |
| (SEQ ID NO: 20) | GGCCTCTATGGAGCA | hNUDT2 into pCI-neo |
| | AA | plasmid |
| WH-Oligo-413 | AAActcgagATGGCG | Forward primer for cloning |
| (SEQ ID NO: 21) | GCCGTGCAGGC | hLysRS into pCI-neo |
| | | plasmid |
| WH-Oligo-414 | AAAgcggccgcCTAG | Reverse primer for cloning |
| (SEQ ID NO: 22) | ACAGAAGTGCCAACT | hLysRS into pCI-neo plasmid |
| | GTTGTGC | |
| WH-Oligo-401 | GCAGACCCAACAGCA | Forward primer for making |
| (SEQ ID NO: 23) | AGCAGGCATAGAAGC | NUDT2 catalytic dead point |
| | AGGCC | mutations |
| WH-Oligo-402 | TGCTGTTGGGTCTGC | Reverse primer for making |
| (SEQ ID NO: 24) | CTCAGGGCTGTTTCC | NUDT2 catalytic dead point |
| | AAGTC | mutations |
| WH-Oligo-413 | AAActcgagATGGCG | Forward primer for making |
| (SEQ ID NO: 21) | GCCGTGCAGGC | hLysRS(G540Y) point |
| | | mutations |
| WH-Oligo-414 | AAAgcggccgcCTAG | Reverse primer for making |
| (SEQ ID NO: 22) | ACAGAAGTGCCAACT | hLysRS(G540Y) point |
| | GTTGTGC | mutations |
| qRT-PCR for | | |
| transfected | | |
| RNAS | | |
| WH-Oligo-436 | CTTCGCTCTGTGCTC | Realtime qRT-PCR primer |
| (SEQ ID NO: 25) | GTGT | for detecting transfected |
| | | capped RNA, forward |
| WH-Oligo-437 | GAACAGGAAAGCACC | Realtime qRT-PCR primer |
| (SEQ ID NO: 26) | ACGGA | for detecting transfected |
| | | capped RNA, reverse |
| WH-Oligo-364 | GTTGTCTCCTGCGAC | Mouse GAPDH qRT-PCR |
| (SEQ ID NO: 27) | TTCA | forward primer |
| WH-Oligo-365 | GGTGGTCCAGGGTTT | Mouse GAPDH qRT-PCR |
| (SEQ ID NO: 28) | CTTA | reverse primer |
| Other Nudt | | |
| mouse mutants | | |
| generation | | |
| WH-Oligo27 | GAAATTAATACGACT | CRISPR F primer-1 targeting |
| (SEQ ID NO: 29) | CACTATAGGGGACGC | mNUDT16(gene specific |
| | GCAGGACAGCTGCCG | sequence underlined) |
| | TTTTAGAGCTAGAAA | |
| | TAGC | |
| WH-Oligo28 | GAAATTAATACGACT | CRISPR F primer-2 targeting |
| (SEQ ID NO: 30) | CACTATAGGGTCAGA | mNUDT16(gene specific |
| | GCGTTCAACGCGGAG | sequence underlined) |
| | TTTTAGAGCTAGAAA | |
| | TAGC | |
| WH-Oligo95 | GATGCAGATGCGCTT | Forward primer for NUDT16 KO |
| (SEQ ID NO: 31) | TGA | mouse genotyping |
| WH-Oligo97 | CTCCAGCCCATGGTC | Reverse primer for NUDT16 KO |
| (SEQ ID NO: 32) | C | mouse genotyping |
| WH-Oligo33 | GAAATTAATACGACT | CRISPR F primer-1 targeting |
| (SEQ ID NO: 33) | CACTATAGGCGATGG | mNUDT3(gene specific |
| | ATTGTCCCCGGAGGG | sequence underlined) |
| | TTTTAGAGCTAGAAA | |
| | TAGC | |
| WH-Oligo34 | GAAATTAATACGACT | CRISPR F primer-2 targeting |
| (SEQ ID NO: 34) | CACTATAGGCGTGCT | mNUDT3(gene specific |
| | GATGACCTGTCCTGG | sequence underlined) |
| | TTTTAGAGCTAGAAA | |
| | TAGC | |
| WH-Oligo101 | GGTTCTTAGTTACTG | Forward primer for NUDT3 KO |
| (SEQ ID NO: 35) | TGGTGC | mouse genotyping |
| WH-Oligo103 | TGTCTTTCAGGGTGA | Reverse primer for NUDT3 KO |
| (SEQ ID NO: 36) | CGAG | mouse genotyping |
| WH-Oligo36 | GAAATTAATACGACT | CRISPR F primer-1 targeting |
| (SEQ ID NO: 37) | CACTATAGGAGACAA | mNUDT12(gene specific |
| | TAGAAGATGCTGTGG | sequence underlined) |
| | TTTTAGAGCTAGAAA | |
| | TAGC | |
| WH-Oligo37 | GAAATTAATACGACT | CRISPR F primer-2 targeting |
| (SEQ ID NO: 38) | CACTATAGGAGTATG | mNUDT12(gene specific |
| | TCTCTTGTCAACCAG | sequence underlined) |
| | TTTTAGAGCTAGAAA | |
| | TAGC | |
| WH-Oligo104 | ATTGAATGGGGGCTC | Forward primer for NUDT12 KO |
| (SEQ ID NO: 39) | TTTC | mouse genotyping |
| WH-Oligo106 | CTATTTCATTCTTGT | Reverse primer for NUDT12 KO |
| (SEQ ID NO: 40) | CAACTTTAATTTCTG | mouse genotyping |
| | TA | |
| WH-Oligo39 | GAAATTAATACGACT | CRISPR F primer-1 targeting |
| (SEQ ID NO: 41) | CACTATAGGGCATTC | mNUDT15(gene specific |
| | TTCCCAGGTCTCACG | sequence underlined) |
| | TTTTAGAGCTAGAAA | |
| | TAGC | |
| WH-Oligo40 | GAAATTAATACGACT | CRISPR F primer-2 targeting |
| (SEQ ID NO: 42) | CACTATAGGAAATGT | mNUDT15(gene specific |
| | GTGCTTTGCCTCCGG | sequence underlined) |
| | TTTTAGAGCTAGAAA | |
| | TAGC | |
| WH-Oligo107 | CAGTCTAAAGCATAA | Forward primer for NUDT15 KO |
| (SEQ ID NO: 43) | ACTAATTACCATTG | mouse genotyping |
| WH-Oligo109 | CCTCGGCTCCGAATC | Reverse primer for NUDT15 KO |
| (SEQ ID NO: 44) | AT | mouse genotyping |
| WH-Oligo42 | GAAATTAATACGACT | CRISPR F primer-1 targeting |
| (SEQ ID NO: 45) | CACTATAGGGACTTC | mNUDT17(gene specific |
| | CAGATACTGGAGTGG | sequence underlined) |
| | TTTTAGAGCTAGAAA | |
| | TAGC | |
| WH-Oligo43 | GAAATTAATACGACT | CRISPR F primer-2 targeting |
| (SEQ ID NO: 46) | CACTATAGGTTGTGT | mNUDT17(gene specific |
| | CCTTCTGGGGTTATG | sequence underlined) |
| | TTTTAGAGCTAGAAA | |
| | TAGC | |
| WH-Oligo110 | TCACCTTGGGTGTGG | Forward primer for NUDT17 KO |
| (SEQ ID NO: 47) | G | mouse genotyping |
| WH-Oligo112 | ATGACTTGGGGACTC | Reverse primer for NUDT17 KO |
| (SEQ ID NO: 48) | CAG | mouse genotyping |
| WH-Oligo45 | GAAATTAATACGACT | CRISPR F primer-1 targeting |
| (SEQ ID NO: 49) | CACTATAGGCGTCAT | mNUDT19(gene specific |
| | CGGGCAGCGCCGCGG | sequence underlined) |
| | TTTTAGAGCTAGAAA | |
| | TAGC | |
| WH-Oligo46 | GAAATTAATACGACT | CRISPR F primer-2 targeting |
| (SEQ ID NO: 50) | CACTATAGGGCTCCT | mNUDT19(gene specific |
| | GCGGCCGCGGGACGG | sequence underlined) |
| | TTTTAGAGCTAGAAA | |
| | TAGC | |
| WH-Oligo113 | gagccccctcggcaa | Forward primer for NUDT19 KO |
| (SEQ ID NO: 51) | cccc | mouse genotyping |
| WH-Oligo115 | caggccggccggagg | Reverse primer for NUDT19 KO |
| (SEQ ID NO: 52) | cgaca | mouse genotyping |

Forward (F) primer design template (SEQ ID NO: 53):

5'-GAAATTAATACGACTCACTATAGGNNNNNNNNNNNNN
NNNNNNNGTTTTAGAGCTAGAAATAGC-3'

N represent the gene-specific sequence.

The following components were mixed to prepare the PCR reaction: 20 μl 5× Phusion HF buffer, 67 μl ddH2O, 2 μl 10 mM dNTPs, 5 μl of 10 μM CRISPR F primer, 5 μl of 10 μM CRISPR sgR primer, and 1 μl Phusion DNA polymerase. The PCR reaction was set as follows: 98° C. for 30 s, 35 cycles of [98° C. for 10 s, 60° C. for 30 s and 72° C. for 15 s], 72° C. for 10 min, and finally at 4° C. to hold the reaction. The PCR product (˜110 bp) was agarose gel-purified using mini-elute gel extraction kit (Qiagen, Cat. No. 28604). The purified DNA was used to produce gRNA by in vitro transcription via the T7 promoter. In vitro transcription was carried out with the MEGAshortscript™ T7 Transcription Kit (Life technologies, Cat. No. AM1354) for 4 hours at 37° C. Reactions were treated with DNase I to remove template DNA, phenol-chloroform extracted and precipitated with ethanol. Quality of the generated gRNA was verified by 1.5% agarose gel electrophoresis.

Denaturing formaldehyde-agarose gel electrophoresis: Quality of generated gRNAs were verified by 1.5% agarose-formaldehyde gel electrophoresis. Agarose gel was prepared by mixing 0.75 g agarose, 36.5 ml H2O, 5 ml of 10×MOPS buffer (0.2 M MOPS, 80 mM sodium acetate, 10 mM EDTA) and 8.5 ml of 37% formaldehyde. Approximately, 4 μg of RNA was dissolved in the 4×RNA loading buffer (50% formamide, 6.5% formaldehyde, MOPS buffer 1×, bromophenol blue 0.2%, ethidium bromide 50 μg/ml) and heated to 65° C. for 10 min. RNA was loaded into the gel and run at 70V for approximately 90 minutes. Gel was imaged in the E-Box VX5 (Vilber Lourmat, France) UV transilluminator.

Preparation of injection mix: We mixed 12.5 ng/μl of each gRNA with 25 ng/μl of Cas9 mRNA (Thermo Scientific, Cat. No. A29378), in injection buffer (10 mM Tris pH 7.5, 1 mM EDTA, pH 8.0). Aliquots of 20 μl were prepared and stored at −80° C.

Injection of mouse embryos of the hybrid background B6D2F1/J (black coat colour) was carried out at the Transgenic Mouse Core Facility, University Medical Centre (CMU), University of Geneva. The B6D2F1/J hybrid line (also called B6D2; The Jackson Laboratory, stock no. 100006) is a cross between C57BL/6J (B6) and DBA/2J (D2), and heterozygous for all B6 and D2 alleles. The NMRI (Naval Medical Research Institute) mice, which have a white coat colour were used as foster mothers.

### Genotyping

Ear-punches of the weaned animals (21 days-old) or toe-clips of pups (7 days-old) were digested in 100 μL of buffer containing 10 mM NaOH, 0.1 mM EDTA for 120 min at 95° C. After centrifugation at 3000 rpm for 10 min, 50 μL of the supernatant was transferred to a new tube containing 50 μL of TE buffer (20 mM Tris-HCl, pH 8.0 and 0.1 mM EDTA). An aliquot of 2 μL of the digestion mix was used for PCR verification. Primers to genotype the knockout allele were WH-Oligo98 and WH-Oligo100 (Table 2). The expected sizes of PCR products are: Nudt2^{+/+} (280 bp, WT) and Nudt2^{-/-} (213 bp, knockout). Identity of the bands was confirmed by Sanger sequencing.

Reaction mix for 20 μL PCR reactions: 10 μL of Phire Green Hot Start II PCR Master Mix (Thermo Scientific, Cat. No. F126L), 1.0 μL primer mix (stock 10 nM each), 2.0 μL ear or toe DNA (100-200 ng) solution, and 7.0 μL water to make 20 μL final volume. PCR reactions were carried out using the following conditions: 98° C. for 30 s, 35 cycles of [98° C. for 5 s, 55° C. for 5 s and 72° C. for 10 s], 72° C. for 1 min, and finally at 4° C. to hold the reaction. Reactions were examined by 1.5% agarose gel electrophoresis (FIG. 5C).

### Clones and Constructs

The mouse NUDT2 protein depicted in the cartoons is based on the sequence from NCBI (NCBI: NP_079815.2). Coding sequences for full-length (FL) human NUDT2 (147 aa; GenBank: AAH04926.1) and mouse NUDT2 (147 aa; GenBank: AAH25153.1) were amplified from human HeLa cell total RNA or from mouse liver total RNA, respectively, by reverse transcription-PCR (RT-PCR). Coding sequence for FL human Lys-tRNA synthetase (hLysRS) (597 aa; GenBank: AAH04132.1) was amplified from human HeLa cell total RNA by reverse transcription-PCR (RT-PCR). The point mutant (G540Y) version of hLysRS that has a higher capacity to make Ap4A (1) was prepared by mutagenesis. The coding sequence for mouse DCP2 (422 aa; GenBank: BAB30946.1) catalytic core domain (mDCP2, 95-260 aa) was amplified by RT-PCR from mouse spleen total RNA. The expression construct for FL human NUDT12 is described (2). For expression in mammalian cells, the required coding sequences were cloned into the mammalian expression vector (pCI-neo vector backbone) allowing production of fusion proteins with an N-terminal 3×FLAG-HA tag from a cytomegalovirus (CMV) promoter. Sequence of the N-terminal 3×FLAG-HA tag (in capital letters with a spacer between them) is as follows:

(SEQ ID NO: 54)
ATGGACTACAAAGACCATGACGGTGATTATAAAGATCATGATA
TCGATTACAAGGATGACGATGACAAGggcggcagcggcTACCC
ATATGATGTTCCAGATTACGCT

### Constructs for Recombinant Protein Production in Prokaryotic Expression Systems

To express mouse DCP2 catalytic core protein (mDCP2, 95-260 aa) and 6×His-SUMO-tagged FL human NUDT2, the amplified cDNA was cloned into pE™-11SS vector to express as 6×His-Strep-SUMO fusion protein by Gibson assembly (NEB, cat. no. E2611S) at the restriction sites NcoI/KpnI.

To express 6×-His-tagged FL human and mouse NUDT2 protein, Nudt2 cDNA was cloned into the pET-28a bacterial vector by BamHI/NotI sites with N-terminal 6×-His tag. The plasmids were transformed into the E. coli BL21 (DE3) pLysS strain, and the cultures were grown at 37° C. and induced with 0.6 mM Isopropyl β-D-1-thiogalactopyranoside (IPTG) when the culture density reached 0.7 (OD600). With the incubation at 20° C. for 18 hr, the cells were collected by centrifugation (4000×g, 20 min, 4° C.).

### Constructs for Recombinant Protein Production in Insect Expression Systems

For insect cell expression of full-length human Lys-tRNA synthetase G540Y mutant (hLysRS^{G540Y}), the coding sequence was cloned into pACEBac2S vector for the expression as N-terminal His-Strep-SUMO fusion protein. The constructs used for recombinant protein production were verified by restriction digest, as well as by Sanger sequencing.

### Recombinant Protein Production

Production of hLysRS^{G540Y} mutant was carried out in insect cell lines using the baculovirus expression system. The insect ovary-derived cell lines used are: High Five (Hi5) insect cell line originating from the cabbage looper (Trichoplusia ni) and the Sf9 cells derived from the fall army worm Spodoptera frugiperda. Briefly, the hLysRS^{G540Y} coding sequence was cloned into pACEBac2S vector. Plasmids carrying the target gene were transformed into DH10EMBacY competent cells for recombination with the baculovirus genomic DNA (bacmid). The bacmid DNA was extracted and transfected with FuGENE HD (Promega, cat. no. E231A) into the Sf9 insect cells for virus production. The supernatant (V₀) containing the recombinant baculovirus was collected after 48 to 72 h post-transfection. To expand the virus pool, 3.0 mL of the V₀ virus stock was added into 25 mL of Sf9 (0.5×10⁶/mL) cells. The resulting cell culture supernatant (V₁) was collected 24 h post-proliferation arrest. For large-scale expression of the protein, Hi5 cells were infected with virus (V₁) and cells were harvested 72 h after infection.

For mouse DCP2 catalytic core (mDCP2, 95-260 aa), 6×His-SUMO-tagged FL human NUDT2, and 6×His-human NUDT2 or 6×His-mouse NUDT2 expression, plasmids were transformed into the E. coli BL21 (DE3) strain and grown in LB media. Protein expression was induced by addition of 0.5 mM Isopropyl β-D-1-thiogalactopyranoside (IPTG) when the culture density reached around 0.8 (OD600). The proteins were then expressed overnight at 20° C. following induction.

Purification of hLysRS^{G540Y}, mDCP2 (95-260 Aa) and 6×his-SUMO-Tagged Human NUDT2 Proteins

The cells (bacteria or insect cells) were collected by centrifugation and lysed by sonication in lysis buffer: 50 mM Tris-HCl, pH 8.0, 300 mM NaCl, 5% Glycerol, 5 mM 2-mercaptoethanol, 40 mM Imidazole and EDTA-free protease inhibitor (Thermo Scientific, Cat. No. PI88666). The lysate was centrifuged at 18,000 rpm for 50 minutes at 4° C. Supernatant was collected and incubated with with Ni²⁺ chelating sepharose FF beads for two hours in the cold room. After incubation, the beads were washed by imidazole gradient washing buffer (40 mM-50 mM-60 mM) and finally bound protein was eluted with 250 mM imidazole in lysis buffer. For hLysRS^{G540Y} and mDCP2 (95-260 aa), the N-terminal tag was cleaved by the TEV protease at 4° C. overnight in the dialysis buffer (50 mM Tris-HCl pH 8.0, 300 mM NaCl, 5 mM 2-mercaptoethanol). The cleaved tag was removed by a second purification on Nickel beads. All proteins (tagged or untagged) were further purified by gel-filtration chromatography: Superdex 200 column (GE Healthcare, Cat. No. 28990944) for hLysRS^{G540Y} and Superdex 75 column (GE Healthcare, Cat. No. 29148721) for mDCP2 (95-260, aa). The columns were equilibrated in the buffer (25 mM Tris-HCl pH 8.0, 150 mM KCl, 5% glycerol and 1 mM DTT).

The pure fractions containing untagged hLysRS^{G540Y} or mDCP2 (95-260 aa) or 6×His-SUMO-tagged FL human NUDT2 were verified by SDS-PAGE electrophoresis (FIG. 9C) and used for biochemical assays.

### Purification of Human and Mouse NUDT2 and Human NUDT12

For the purification of 6×His-tagged human and mouse NUDT2, the bacteria cells were lysed by sonication in lysis buffer containing 50 mM Tris-HCl pH 8.0, 400 mM NaCl, 5% glycerol, 5 mM 2-mercaptoethanol, 40 mM imidazole and protease inhibitor. After the centrifugation, the supernatant was incubated with Ni-NTA bead for 2 hr in the cold room. The bead was then washed with 20× volume of lysis buffer and eluted with lysis buffer with 250 mM imidazole. The 6×His-tagged protein was further polished by the gel-filtration chromatography (Superdex 75, GE Healthcare) in the storage buffer (50 mM Tris-HCl pH 8.0, 150 mM NaCl, 10% glycerol, 2 mM DTT).

We used the tagged 6×-His-hNUDT2 (FIG. 9A) for most biochemical experiments in this study. The mouse fusion protein was used for verification of activity and found to be identical to the human protein. Some experiments also used tagged 6×His-SUMO-hNUDT2 (FIG. 9C). Untagged human NUDT12 was purified as described previously (2)

### Antibodies

### Commercial Antibodies

Primary antibodies: mouse anti-RSAD2 (Sigma-Aldrich, Cat. No. MABF106); rabbit anti-TUBULIN (Cell Signaling Technology, Cat. No. 2148) and rabbit anti-PARK7 (ThermoFisher, Cat. No. PA5-13404).

Secondary antibodies: For Western blot analyses, the following secondary antibodies conjugated to Horse Radish Peroxidase were used: anti-rabbit IgG HRP-linked (GE Healthcare, Cat. No. NA934), anti-mouse IgG HRP-linked (GE Healthcare, Cat. No. NA931).

Antibodies Generated for this Study

We generated rabbit polyclonal antibodies to human NUDT2. Two New Zealand White (NZW) rabbits were immunized with soluble antigens (Biotem, France). The antigen used was the purified untagged full-length human NUDT2 protein produced in bacteria cells. For each injection, 1 mg/mL protein was used. After six injections (at day 0, 14, 28, 56, 70 and 89) crude immune serum was collected (at day 39, 67, 82 and 96) and frozen. Anti-NUDT2 rabbit polyclonal antibodies were purified using recombinant human NUDT2 protein. The antibody was useful for detecting the mouse protein. The crude sera and the affinity-purified antibodies detect a strong band in different mouse organ lysates by Western analysis (FIG. 1B-C, FIG. 5A).

### Antibody Purification

To purify antibodies against human NUDT2, large amount of recombinant untagged full-length human NUDT2 protein was resolved via SDS-PAGE and semi-dry transferred onto a nitrocellulose membrane. After reversible staining with Ponceau S (Sigma-Aldrich, Cat. No. P3504), part of the membrane containing the antigen was cut out and incubated overnight with 8 mL crude immune sera. After washes (1×PBS), bound antibodies were eluted with 500 μL (elute only once to keep it concentrated) of low pH solution (0.1 M Glycine pH 2.5, 150 mM NaCl) and immediately neutralized with 150 μL of neutralization buffer (0.5 M Tris-HCl pH 8.0, 150 mM NaCl, determine volume required by tests just before use). Antibodies were concentrated and stored in 1×PBS, 50% glycerol at −20° C.

### Establishment of Mouse Embryonic Fibroblasts (MEFs) Cell Culture

MEFs were isolated from E14.5 embryos and set up as cell culture following the method described before (3). Briefly, male and female mice were crossed to produce embryos for MEFs. Formation of the copulatory plug was monitored, and after 14 days, the females were anesthetized by 2.5% avertin, and euthanized by cervical dislocation. Uterus was isolated and transferred to 50 mL tubes containing 30 mL sterile PBS, invert the tube several times to wash the uterus. In a laminar flow cabinet, open the 50 ml tube and transfer the washed uterus with embryos into a 10 cm tissue culture dish containing 10 ml sterile PBS. Cut through one side of the uterine wall to expose individual embryos. Open the yolk sac and dissect out all the embryos. Transfer all embryos into a new dish containing 10 ml sterile PBS, remove liver, heart, and brain, take one piece for genotyping.

Wash the remaining part of the embryo in sterile PBS to remove as much blood as possible, then transfer the embryos into a new dish. Add 2 to 3 ml of ice-cold 0.25% trypsin-EDTA (ThermoFisher, Cat. No. 25200056) to the dish, and tease all embryos into fine pieces, then transfer them into a 15 mL tube containing 3 mL ice-cold 0.25% trypsin-EDTA (one tube for each embryo). Let the tube stand at 4° C. overnight. On the next morning, aspirate off most of the trypsin solution leaving an amount equivalent to approximately two volumes of the tissue. Cap the tube and incubate 30 min in a 37° C. water bath. Add 8 mL MEF culture medium [Dulbecco's modified Eagle medium (DMEM) (ThermoFisher, Cat. No. 21969-035) containing 10% 10% (v/v) fetal bovine serum (FBS) (ThermoFisher, Cat. No. 26140079), 0.1 mM β-mercaptoethanol (tissue culture grade) (Merck, Cat. No. M3148-100ML), 50 U penicillin, 50 μg/ml streptomycin (ThermoFisher, Cat. No. 15140122)] to each tube, pipet vigorously and repeatedly up and down to break up the digested tissues into a cell suspension. Let the tube stand still for 1 min to let remaining clumps fall to the bottom of the tube, transfer the supernatant to a new 15 mL tube. Add more MEF culture medium to the tube containing the remaining tissue clumps, repeat the pipetting step, and stand once more, and collect the supernatant. Mix the cell suspension and plate the cells in 10 cm tissue culture dishes. The next day, replace MEF culture medium. The established MEFs can be used for 5-6 generation.

### In Vitro Transcription of RNAs for Cell Transfections

The commonly used T7 class III promoter ϕ6.5 initiates using GTP, while the class II promoter ϕ2.5 initiates with ATP (4). We prepared DNA templates containing the T7 class II promoter ϕ2.5 at the 5′ end, with the transcription start site adenosine, followed by residues composed of T, C and G (there is no adenosine except at the TSS position). The template was either amplified by PCR or annealed to generate the dsDNA template for in vitro transcription (Table 2).

For the 200 nt template, the required sequence was commercially synthesized and received as an insert within the pUC57 plasmid (Genewiz). The primers WH-Oligo-297 and WH-Oligo-298 (Table 2) were used to amplify the sequence and purified by gel elution using Qiagen Gel Extraction kit (Qiagen, Cat. No. 28706). For the 50 nt template, two single-stranded DNA oligos (WH-Oligo-421 and WH-Oligo-424) were mixed and annealed at 10 μM concentrations, by heating to 95° C. for 5 min (Thermomixer, Eppendorf). The tubes with the oligos were then allowed to slowly cool down to room temperature by turning off the Thermomixer. Oligo sequences are listed in Table 2. All DNA oligonucleotides were purchased from Thermofisher Scientific.

The 200 nt or 50 nt RNA with different 5′ end caps were synthesized using MEGAshortscript™ T7 Transcription Kit (Life technologies; Cat. No. AM1354). The 5′ triphosphate (Tri-pho)-RNA was made according to the protocol provided in the kit, while for making Ap4A RNA, 75 mM Ap4A (Jena Bioscience, Cat. No. NU-507-25) was added into the nucleotide solution instead of ATP. For making m⁷G cap0 RNA and m⁷G cap1 RNA, 75 mM of a mixture of ATP+m⁷GpppA (NEB; Cat. No. S1405S) or ATP+m⁷GpppAₘG (Trilink, Cat. No. N-7113-1) was used with a ratio 1:4 instead of 75 mM pure ATP.

To get rid of any unintended 5′ triphosphate RNA synthesized together with the capped RNAs, we treated the RNAs with 5′-polyphosphatase (Lucigen, Cat. No. RP8092H) and Terminator 5′-Phosphate-Dependent Exonuclease (Lucigen, Cat. No. TER51020). This converts the 5′ triphosphate end to one with a 5′ monophosphate, which is then degraded by the exonuclease. This was followed by purification with RNA Clean & Concentrator-5 (ZYMO Research, Cat. No. R1014). This step is important before using the RNAs for cell transfections to study production of the type I interferon, as the contaminating 5′ triphosphate end is a potent trigger.

DNA template for making the 200 nt RNA is given below:

(SEQ ID NO: 55)
5'CAGTAATACGACTCACTATTAGTCCGTCCGCCGCCCTTGCT
GGTCTTTCTCGCCCGCTGCTGCCTGCCTGCGCTTCGCTCTGTG
CTCGTGTCCCGTCGCGTTGGGCTCCCTGGCTTCTGGGTTTCCT
TCTTGGTGCGTGCGCCTCCGGTGGTGTGCTCGTTCCGTGGTGC
TTTCCTGTTCTCGGGTCGCGGTGTTTGTTCGGGTCTGCCTTTG
GTGGGTT

DNA template for making the 50 nt RNA is given below:

(SEQ ID NO: 56)
5'CAGTAATACGACTCACTATTAGTCCGTCCGCCGCCCTTGCT
GGTCTTTCTCGCCCGCTGCTGCCTGCCTG

The T7 ϕ2.5 promoter is underlined. The transcription start site (TSS) adenosine is in bold.

### ELISA Assay for Detecting Interferons (IFN)

### Sample Preparation

Mouse Serum: All blood samples from Nudt2^{+/+} or Nudt2^{-/-} adult mice were isolated and allowed to clot by incubation for 2 h at room temperature. After centrifugation for 20 min at 2000×g, the serum was collected and aliquoted. These were immediately used for ELISA or stored at −20° C. for future use.

Cell culture supernatants: MEFs (in 12-well plates, 70% confluent) were transfected with different capped RNAs (2 nM), or different amount of Ap4A RNAs as indicated in the figure (FIG. 4). The transfection was done by using Lipofectamine™ 3000 Transfection Reagent (Invitrogen; L3000015). Briefly, appropriate amount of RNAs were diluted in 25 μL Opti-MEM™ Medium (ThermoFisher; 11058021), twice the amount of Lipofectamine 3000 as RNAs was also diluted in 25 μL Opti-MEM™ Medium. After 5 min, mix them and vortex vigorously for 15 seconds and incubate for another 15 min. Next, transfer the mixed reagents onto cells in the DMEM CM. After 24 h of transfection, the supernatants (containing secreted interferons) were removed, clarified by centrifugation and immediately used for ELISA. Alternatively, it was aliquoted and stored at −20° C. for future use.

For each RNA, we transfected two independent biological replicates of MEFs, each sitting in duplicate wells (of a 12-well plate). Thus, there are four transfections for each RNA sample: two biological replicates, each with two technical replicates. We then measured IFN levels in the supernatant from each well by carrying out ELISA measurements in technical duplicates. The data from the four transfected wells were plotted (FIG. 4).

### ELISA

To detect mouse interferons (IFN), we used the following kits: Mouse IFN-α ELISA Kit (R&D system; Cat. No. 42120-1) and mouse IFN-β ELISA Kit (R&D system, Cat. No. 42400-1). The protocol provided by the manufacturer was followed. A standard curve with recombinant mouse IFN proteins (Mu-IFN-α and IFN-β) provided in the kit is run on the same plate. This ELISA is based on a sandwich immunoassay, where after incubation with the primary antibodies recognizing the IFN, a secondary antibody conjugated to horseradish peroxidase (HRP) is added. Tetramethyl-benzidine (TMB) is used as a substrate.

For IFN-α, 100 μL of standards (mouse IFN-α), blanks or test samples were added to the plates provided with the kit. Each sample was processed in technical duplicates. After addition of 50 μL of the diluted antibody (1:1000), the samples were incubated at room-temperature (RT) for 1 hour with 450 rpm shaking, following which the plates were transferred to 4° C. for further 24-hour incubation. Subsequently, the solution was aspirated, washed 4 times with washing buffer provided by the kit, and 100 μL of diluted HRP solution (1:175) was added. After incubation for 2 h at RT with 450 rpm shaking, the solution was aspirated, and washed again 4 times with the washing buffer. The reaction was developed by addition of the TMB substrate, and the plates were immediately read at 450 nm absorbance by Typhoon FLA 9500 scanner (GE Health). Essentially similar protocol with slight changes was used for detecting IFN-β.

All the IFN ELISA experiments at least had 3 biologically replicates (individual mice or different MEF transfections), with each measurement being done in technical duplicates.

### Synthesis of Radioactive Ap4A

To produce radioactive Ap4A, we used the demonstrated capacity of tRNA synthetase, in particular the lysyl tRNA synthetase, to generate Ap4A as a mis-reaction (5). The mutant (G540Y) human Lys-tRNA synthetase is demonstrated to have a higher Ap4A generation capacity (1). To prepare radioactive Ap4A, we incubated 60 μCi of radioactive α-³²P-ATP (Perkin Elmer, Cat. no. BLU003X250UC; 75 fmol in 20 ul or 3.75 nM concentration) with 1 μM recombinant human Lys-tRNA synthetase (G540Y mutant) at 37° C. overnight in a 20 μl solution [100 mM Tris-HCl, pH 8.0, 10 mM MgCl2, 0.1 mM ZnCl2, 1 mM Lysine and 0.1 units inorganic pyrophosphatase (Sigma-Aldrich, Cat. no. I1643)].

We resolved the reaction products using thin-layer chromatography, using PEI-cellulose TLC plates (Merk, Cat. No. 1057250001) and developing it in 0.45 M (NH₄)₂SO₄ using a glass chamber at room temperature. In addition to the ATP already present in the reaction, we see the presence of Ap4A (FIG. 9D). From this Ap4A mix (ATP and Ap4A), we obtained purified radioactive Ap4A (FIG. 9E) by HPLC (purification service kindly provided by Hartmann Analytic, Germany). For some experiments (FIG. 9F-G) we used this Ap4A mix (ATP and Ap4A). However, for most experiments (FIG. 2, FIG. 9E) the purified radioactive Ap4A was used.

Preparation of RNA with Labelled Ap4A Cap

We produced in vitro transcribed Ap4A-capped RNAs that are cap-labelled with radioactivity (32P) using either the Ap4A mix (ATP and Ap4A) or with purified Ap4A. The Ap4A is double-labelled: AppppA, bold indicates 32P label.

Briefly, using the Ap4A mixture (ATP and Ap4A) or Ap4A instead of ATP for in vitro transcription by MEGAshortscript™ T7 Transcription Kit (Life technologies; Cat. No. AM1354), we produced a 50 nt RNA. Template is the same as making 50 nt RNA for MEFs transfection. Note that if the mixture is used, this nucleotide mixture (ATP and Ap4A) still has all the components (lysine, inorganic pyrophosphatase etc) added during the Ap4A synthesis. In vitro transcription reaction was carried out at 37° C. for at least 4 h in a 20 μL reaction containing 6 pmol DNA template, 2 μL 10× reaction buffer, 2 μL of nucleotides (75 mM CTP, 75 mM UTP, 75 mM GTP), 2 μL of ATP/Ap4A mixture that we produced above, and 2 μL T7 enzyme mix (MEGAshortscript™ Kit, Life technologies, Cat. No. AM1354). After that, 1 μL TURBO DNase was added and incubated at 37° C. for 15 min to remove the DNA template. Unincorporated nucleotides were removed from the RNA preparation by Microspin G-25 Column (GE Healthcare, Cat. No. 27-5325-01), and the process was repeated twice. The radioactively labelled RNA was separated by 10% urea gel. The gel was briefly (30 min to 2 h) exposed to Storage Phosphor screens (GE Health) and scanned with a Typhoon FLA 9500 scanner (GE Health).

We observed three distinct RNA bands when using the Ap4A mixture to initiate the transcription (data not shown). We expect two of the three bands to correspond to RNA starting with ATP or Ap4A, while the identity of the third RNA band is unknown. We isolated individual bands using labelled positional markers. RNAs were eluted overnight in elution buffer (300 mM NaCl) at 25° C., with shaking at 750 rpm on a Thermomixer (Eppendorf). The following day, the eluted labelled RNA was precipitated with ethanol and glycogen at −20° C. for 30 min. Subsequently, radioactive RNA was collected by centrifugation at 4° C. for 15 min and dissolved in water. Based on RNA decapping assay with NUDT2, we identified one of the bands as Ap4A-capped RNA (data not shown). If purified Ap4A is used, only one band is observed in the urea-PAGE. RNA prepared with such purified Ap4A was used for most experiments.

Preparation of RNA with labelled m⁷G cap

The ³²P-labeled m⁷G-capped RNA was generated by in vitro capping using the vaccinia virus capping enzyme (New England BioLabs, Cat. No. M2080S). We first prepared by in vitro transcription unlabelled 50 nt RNA with a 5′ triphosphate end using the same template as described above. Capping reaction (20 μL) included 100 ng 5′ triphosphate RNA, 2 μL 10× capping buffer (New England BioLabs, Cat. No. M2080S), 1 μL Ribolock RNase inhibitor (ThermoFisher, Cat. No. EO0382), 60 μCi α-³²P-GTP (Perkin Elmer, Cat. No. BLU006H), 0.2 mM S-adenosylmethionine (New England BioLabs, Cat. No. B9003S), 1 μL vaccinia virus capping enzyme (New England BioLabs, Cat. No. M2080S). Reactions were incubated at 37° C. overnight. The next day, labelled RNA was twice purified with Microspin G-25 Columns (GE Healthcare, Cat. No. 27-5325-01), and further separated by 10% urea gel electrophoresis. The radioactive m7G-capped RNA was extracted from the gel in 300 mM NaCl, precipitated with ethanol, and dissolved in water, as described above.

### In Vitro Decapping and Cleavage Assay

For Ap4A-RNA or m⁷G-RNA decapping assay, all reactions in an experiment contained the same amount of radioactively labelled RNA. Recombinant untagged mouse DCP2 (95-260 aa), 6×His-SUMO-tagged or 6×His-tagged human NUDT2 or untagged human NUDT12 at 250 nM to 750 nM concentrations were used. The decapping reaction (20 μL) contained 100 mM KCl, 2 mM MgCl2, 1 mM MnCl2, 2 mM DTT, 10 mM Tris-HCl (pH 7.5), and was incubated at 37° C. for 30 min. Note that 6×His-SUMO-tagged human NUDT2 was poorly active when compared to 6×His-tagged human NUDT2 (FIG. 9B).

Decapping reaction products were resolved by PEI-cellulose TLC plates (Merk, Cat. No. 1057250001) and developed in 0.45 M (NH4)2SO4 in a glass chamber at room temperature. When the resolving buffer front reached ˜2 cm from the top of the TLC plate, the plates were removed, allowed to air-dry, then wrapped in SaranWrap. Reaction products were visualized by exposure to Storage Phosphor screens (GE Health) and scanned with a Typhoon FLA 9500 scanner (GE Health). When required, signals were quantified by ImageQuantTL.

For hydrolysis assays with Ap4A (AppppA, in bold is 32P), reactions were conducted as above, but the RNA was replaced with appropriate amount of HPLC-purified Ap4A. Hydrolysis assays were conducted with 750 nM of recombinant human NUDT2 or with Nuclease P1 (0.5 unit and 1 unit). NUDT2 hydrolyzes Ap4A (AppppA, in bold is 32P) into α-³²P-AMP and α-³²P-ATP (FIG. 2D). We found that Nuclease P1 also hydrolyzes Ap4A down to AMP (FIG. 9E).

### Mouse Multiple Tissue Western Blot

Multiple tissues were isolated from adult (>60 days) mice. After flash-freezing in liquid nitrogen, a piece of tissue was homogenized in 1 mL lysis buffer [20 mM Tris pH 7.4, 150 mM NaCl, 0.5% Triton X-100, 0.5% sodium deoxycholate, 1 mM DTT, Complete Protease Inhibitor Cocktail Tablet (Roche, Cat. No. 5056489001)]. The lysate was transferred to a 1.5 mL Eppendorf tube, centrifuged at 14000×g for 30 min, and the supernatant collected. An aliquot was taken to measure the concentration by Pierce™ Detergent Compatible Bradford Assay Kit (ThermoFisher, Cat. No. 23246). Lysate concentrations were normalized to 2 μg/μL. The SDS loading buffer was added and boiled at 95° C. for 10 min, and 20 μg of protein per lane was loaded and resolved by SDS-PAGE. Western blot analysis (see below) with purified anti-NUDT2 rabbit polyclonal antibodies was performed. After stripping, the blot was re-probed with anti-PARK7 rabbit polyclonal antibodies, to serve as loading control.

### Western Blot

Whole cell or mouse tissue lysates were separated via SDS-PAGE in order to detect proteins of interest. SDS-PAGE gels were prepared using Ultra-Pure ProtoGel 30% acrylamide (37.5:1) (National Diagnostic, Cat. No. EC-890), ultra-pure water, and resolving gel buffer (0.375 M Tris, 0.1% SDS, pH 8.8) to obtain 8% resolving gel, and with stacking gel buffer (0.125 M Tris, 0.1% SDS, pH 6.8) to obtain 12% stacking gel. N,N,N′,N′-Tetramethylethylendiamin (TEMED; Merck, Cat. No. 1107320100) and 10% ammonium persulfate were added to catalyse the polymerization reaction. Gel electrophoresis was performed at 90 V for 30 min. and then at 120 V for 90 min. After separation, proteins were blotted on the Amersham Protran 0.45 μm nitrocellulose membrane (GE Healthcare, Cat. No. 10600002) overnight at 5 V at room temperature using Trans-Blot SD Semi-Dry Transfer Cell system (Bio-Rad, Cat. No. 1703940). After transfer, membranes were washed with Tris-buffered saline (TBS, 20 mM Tris, 150 mM NaCl, pH 7.6) and blocked for 1 h at room temperature with 5% dry milk in TBS with 0.05% Tween20 (TTBS) (Sigma, Cat. No. P7949). After this, membranes were incubated with primary antibodies for 3 hours at room temperature in 5% milk with TTBS. Then, membranes were washed 3 times for 10 minutes with TTBS and incubated with HRP-conjugated secondary antibody at 1:10 000 dilution, either with anti-rabbit IgG HRP-linked (GE Healthcare, Cat. No. NA934) or anti-mouse IgG HRP-linked (Invitrogen, Cat. No. a27025) for 1 h at room temperature in 5% milk in TTBS. After 1 h, membranes were washed 5 times for 5 min with TTBS and incubated with one of detection reagents: Amersham Prime Western Blotting Detection Reagent (GE Healthcare, Cat. No. RPN2232), SuperSignal West Femto Maximum Sensitivity Substrate (ThermoFisher, Cat. No. 34095) or Pierce ECL 2 Substrate (ThermoFisher, Cat. No. 1896433A) for 5 min. at room temperature. Signal was detected using Amersham Hyperfilm ECL (GE Healthcare, Cat. No. 28906837). The processed films were scanned using Perfection 3200 Photo scanner (Epson) with XSane image scanning software (ver. 0999).

### Histology of Mouse Tissue Sections

### Mouse Tissue Sections

To prepare the paraffin sections, the mouse tissues were washed in PBS, and fixed in 4% paraformaldehyde overnight at 4° C. After washes with PBS, testes were dehydrated in 70% ethanol and stored in 70% ethanol at 4° C. Alternatively, isolated tissues were fixed in Bouin's solution (Sigma, cat. No HT10132) overnight at room temperature and subsequently washed 3 times in 50% ethanol, and in several changes of 70% ethanol, until no yellow dye could be extracted into solution. Samples were sent to Histology core service in University of Geneva, Switzerland where they were further dehydrated in 80%, 90%, 96% and 100% ethanol (90 min for each step), followed by incubation in xylene (3 times 30 min). Xylene was removed and replaced with paraffin and incubated at 56-58° C. Tissues were then transferred into plastic moulds (Polysciences mold S-22; NC0397999) filled with paraffin, and paraffin was allowed to become solid at room temperature. The tissue sections (˜5 μM thickness) were prepared using a microtome. The sections were allowed to stretch at 42° C. and then stored at room temperature.

### HE Staining of Tissue Sections

The slides containing the paraffin sections were placed in a glass slide holder filled with xylene (3×5 min) to remove the paraffin. For rehydration, the slides were incubated in 100% ethanol, 96% ethanol, 70% ethanol, 50% ethanol (2×10 minutes for each step) and miliQ water (2×2 min for each step). Sections were stained with Hematoxylin solution, Harris modified (Sigma-Aldrich, Cat. No. HHS16) for 1-3 min and rinsed in running tap water. To destain the colorant, sections were incubated in acidic alcohol (1% HCl in 70% ethanol) for 5-20 sec and rinsed with water. Then the slides were immersed in bluing solution (ammonium solution) for 15-20 sec and rinsed in tap water. Then, sections were stained with Eosin Y solution with phloxine (Sigma-Aldrich, Cat. No. HT110332) for 20 sec to 1 min and washed with water. For dehydration, the sections were incubated in 70% (10-20 sec), 96% (30 s), 100% ethanol (2 min) and xylene (2 3 5 min). Few drops of Permount (FisherScientific, Cat. No. SP15-100) were deposited on the sections and immediately covered with coverslips. The sections were examined and pictures were taken using widefield (Zeiss Axio Imager Z1 or Axio M2) microscopy.

### Electron Microscopy of Mouse Tissue Sections

Adult mouse testes from Nudt2^{+/+} and Nudt2^{-/-}-males were processed for electron microscopy and images were collected as previously described (2). Mouse testis were fixed in glutaraldehyde solution (EMS, Hatfield, PA) 2.5% in phosphate buffer (PB 0.1 M [pH 7.4]) for 1 h at RT, then washed 3×5 min in phosphate buffer and postfixed in a fresh mixture of osmium tetroxide 1% (EMS) with 1.5% of potassium ferrocyanide (Sigma, St. Louis, MO) in PB buffer for 1 h at RT. The samples were then washed twice in distilled water and dehydrated in acetone solution (Sigma, St Louis, MO, US) at graded concentrations (30%—40 min; 50%—40 min; 70%—40 min; 100%—2×1 h). This was followed by infiltration in Epon resin (EMS, Hatfield, PA, US) at graded concentrations (Epon 33% in acetone—4 h; Epon 66% in acetone—4 h; Epon 100%—2×8 h) and finally polymerized for 48 h at 60° C. in an oven. Ultrathin sections of 50 nm thick were cut using a Leica Ultracut (Leica Mikrosysteme GmbH, Vienna, Austria), picked up on a copper slot grid 2×1 mm (EMS, Hatfield, PA, US) coated with a polystyrene film (Sigma, St Louis, MO, US). Micrographs were taken with a transmission electron microscope FEI CM100 (FEI, Eindhoven, The Netherlands) at an acceleration voltage of 80 kV with a TVIPS TemCamF416 digital camera (TVIPS GmbH, Gauting, Germany) using the software EM-MENU 4.0 (TVIPS GmbH, Gauting, Germany).

### MicroCT

Animals were euthanized and imaged by microCT (Quantum GX microCT, PerkinElemer, Hopkinton, MA, USA), to accurately estimate levels of adipocyte tissues (FIG. 1K and FIG. 7). High resolution microCT images were acquired at 90 kV, 88 μA with a voxel size of 144 μm³ and a field of view of 72 mm×72 mm for 4 min (6). Three scans were needed to acquire the whole bodies of the mice. These scans were then stitched together and analyzed using the VivoQuant™ software version 2020-build9 (inviCRO, Boston MA). Tissues volumetric analysis was conducted using signal-intensity thresholds to classify voxels as skeletal structure (from 190 to 6000 HU), lean (from −150 to 190 HU) and adipose tissues (from −400 to −150 HU). The 3D rendering images show white adipose tissue (WAT) present throughout the body, including the visible visceral fat and those present within tissues (yellow, FIG. 1K and FIG. 7). Due to their positional information, the adipose tissue behind the neck area was identified as the classical interscapular brown adipose tissue (BAT) (blue, FIG. 1K and FIG. 7), and the adipose tissue surrounding the gonads was identified as the epididymal white fat (EWAT) (magenta, FIG. 1K and FIG. 7).

Quantification of the occupied volumes shows that normalized (to body weight) the bone content, lean mass and the overall white fat content are the same for the wildtype control and the Nudt2^{-/-} knockout (KO) (FIG. 7C). When quantified specifically for EWAT/gonadal fat, a slight reduction is seen in the Nudt2^{-/-} KO (FIG. 1K and FIG. 7). In contrast, the BAT is increased in the Nudt2^{-/-} KO (FIG. 1K and FIG. 7). This increase in BAT is further evident in histological sections, where large lipid droplets are present in the interscapular BAT from the Nudt2^{-/-} KO (FIG. 7D).

### Labmaster Experiments

Body mass composition was analyzed using an EchoMRI 700 (EchoMRI, Houston, USA), before and after indirect calorimetric experiment. Body composition was expressed as a percentage of body weight. Mice were analyzed for whole energy expenditure, oxygen consumption and carbon dioxide production, respiratory exchange rate (RER: vCO₂/vO₂), food intake and spontaneous locomotor activity using calorimetric cages (Labmaster, TSE Systems GmbH, Bad Homburg, Germany). Activity was recorded using infrared light beam-based locomotion monitoring system (beam breaks per 15 min). Spontaneous locomotor activity was recorded either with running wheels (Columbus) or inside the cage. Mice were individually housed and acclimatized to the mouse cage four days prior to start of the experimental measurements. Data analysis was carried out with Excel using extracted raw values [vO₂ consumption, vCO₂ production (in mL·h⁻¹), and energy expenditure (kcal·h⁻¹)] from the 5 days of measurements. Subsequently, each value was expressed either per total body weight or whole lean tissue mass extracted from the EchoMRI analysis. Measurements were conducted by Franck Bontems of the Small Animals Phenotyping Platform, CMU, University of Geneva.

### Quantification of RNA Modifications Using LC-MS/MS

Total RNA was isolated from liver, pancreas, spleen, testes, and brain (cortex and cerebellum) of Nudt2^{+/+} and knockout Nudt2^{-/-} mice. Triplicate biological replicates were used.

PolyA⁺ RNA was purified using magnetic Dynabeads™ Oligo (dT) 25 (ThermoFisher, Cat. No. 61005). In brief, total RNA (75 μg) was adjusted to 100 μL with distilled DEPC-treated water. The RNA was heated to 65° C. for 2 min, and placed on ice, to disrupt secondary structures. Dynabeads were resuspended and 200 μL (1 mg) of the beads solution was transferred to a microcentrifuge tube. The tube was placed on the magnet for 30 sec. The supernatant was carefully removed and discarded, then removed from the magnet. About 100 μL Binding Buffer (1 M LiCl₂, 2 mM EDTA, pH 8.0, 20 mM Tris-HCl, pH 7.5) was added to resuspend the beads. Remove supernatant again by placing the tube back on the magnet. After removal of the tube from the magnet, add 100 μL Binding Buffer to the Dynabeads. The total RNA was added to the Dynabeads/Binding Buffer suspension. Mix thoroughly and rotate on a roller or mixer for 3-5 min at room temperature to allow the mRNAs to anneal to the oligo (dT) 25 on the beads. Place the tube on the magnet until the solution is clear. Remove the supernatant. Remove the tube from the magnet and wash the beads twice with 200 μL Washing Buffer B. Remove all the supernatant between each washing steps with the help of the magnet. Elute the RNA with 20 μL 10 mM Tris-HCl, pH 7.5 by heating to 80° C. for 2 min. and place the tube immediately on the magnet to transfer the eluted PolyA⁺ RNA to a new RNase-free tube. For one sample, multiple batches of purification were needed to obtain the 4-12 μg of polyA+ RNA required for each mass spectrometry analysis.

PolyA⁺ RNA (4-12 μg) was hydrolyzed to ribonucleosides by 1 U nuclease P1 (Sigma) in 10 mM ammonium acetate pH 6.0 and 1 mM zinc chloride at 40° C. for 1 h for the analysis of Ap4A. An aliquot was hydrolyzed by 20 U benzonase (Santa Cruz Biotech), 0.2 U nuclease P1 (Sigma) and 0.1 U alkaline phosphatase from E. coli (Sigma) in 10 mM ammonium acetate pH 6.0 and 1 mM magnesium chloride at 40° C. for 1 h for the analysis of unmodified ribonucleosides. The hydrolysates were mixed with 3 volumes of acetonitrile and centrifuged (16000×g, 30 min, 4° C.). The supernatants were lyophilized and dissolved in 8 mM ammonium hydrogencarbonate pH 7.0 for LC-MS/MS analysis of Ap4A and unmodified ribonucleosides. Chromatographic separation of Ap4A was performed using an Agilent 1290 Infinity II UHPLC system with a ZORBAX RRHD Eclipse Plus C18 150×2.1 mm ID (1.8 μm) column (Agilent Technologies). The mobile phase was a gradient of buffer A (8 mM ammonium hydrogencarbonate pH 7.0) and B (methanol) starting with 0.5 min of 5% B, then 3 min of 5-15% B, 3 min of 15-90% B, and finally 4 min re-equilibration with 5% B at flow rate 0.25 ml/min. Unmodified ribonucleosides were chromatographed isocratically with water:methanol:formic acid 80:20:0.1% at 0.25 ml/min. Mass spectrometric detection was performed using an Agilent 6495 Triple Quadrupole system operating in positive electrospray ionization mode, for Ap4A monitoring the mass transitions m/z 837.1-136.1 (CE 50), and 837.1-348.1 (CE 22), and for unmodified ribonucleosides monitoring m/z 268.1-268.1 (A), 284.1-152.1 (G), 244.1-112.1 (C), and 245.1-113.1 (U). We used in vitro synthesized Ap4A RNA as positive control.

We note that the estimated Ap4A levels in the RNA is an underestimation, as we found that Nuclease P1 (used for sample preparation) can hydrolyze Ap4A to AMP (FIG. 9E).

### Metabolomics

Mass spectrometric analyses of metabolites were carried out with tissues (liver, kidney, pancreas and testis) taken from triplicate biological replicates of Nudt2^{+/+} and Nudt2^{-/-} mice. The method followed is a protocol modified from a previous study (7).

Metabolite Extraction from Tissue

Approximately 25 mg of fresh mouse tissue was placed in a homogenizer. Using 500 μL of methanol-water (4:1), the tissue was homogenized using a pistil. The homogenate was transferred to a 2 ml secure lock Eppendorf vial. The remaining lysate was rinsed out by addition of 2×250 μL of fresh methanol-water (4:1) into the homogenizer, and transferred to the same 2 ml secure lock Eppendorf vial, resulting in 1 mL of tissue homogenate. The homogenate was kept on dry ice, while other tissues were processed in a similar manner. The homogenates were centrifuged at 13000 rpm, at 4° C., for 15 min. Subsequently, 900 μL of the clear supernatants were transferred to fresh 1.5 mL Eppendorf tubes. Samples were then shipped (on dry ice) to the Functional Genomics Center Zurich (ETH Zurich) for LC-MS analysis.

### Sample Preparation for LC-MS Analysis

50 μL methanol extract was dried under a nitrogen stream and reconstituted in 20 μL water (MS grade) and diluted with 80 μL injection buffer. The dilution was vortexed and centrifuged (16,000×g, 4° C., 15 min). 50 μL of the supernatant was transferred to a glass vial with narrowed bottom (Total Recovery Vials, Waters) for LC-MS injection. In addition, method blanks, QC standards, and pooled samples were prepared in the same way to serve as quality control for the measurements. Injection buffer was composed of 90 parts of acetonitrile, 9 parts of methanol and 1 part of 5 M ammonium acetate.

### LC-MS Analysis

Metabolites were separated on a nanoAcquity UPLC (Waters) equipped with a BEH Amide capillary column (150 μm×130 mm, 1.7 μm particle size, Waters), applying a gradient of 0.5 mM ammonium acetate in water (A) and 0.5 mM ammonium acetate in acetonitrile (B) from 10% A to 50% A over 10 min. The injection volume was 1 μL. The flow rate was adjusted over the gradient from 3 to 2 μl/min. The UPLC was coupled to Synapt G2Si mass spectrometer (Waters) by a nanoESI source. MS1 (molecular ion) and MS2 (fragment) data was acquired using negative polarization and MSE over a mass range of 50 to 1200 m/z at MS1 and MS2 resolution of >20′000. All biological samples were analyzed at least in triplicate and quality controls were run on pooled samples and reference compound mixtures to determine technical accuracy and stability.

### Targeted Data Analysis

Target metabolites (Ap4A and Gp4A) were quantified by the area under the peak (AUP) of the MS1 extracted ion chromatogram (EIC) of the respective [M-2H]²⁻ ion (m/z of 417.0166 for Ap4A; m/z of 425.0143 for Gp4A) extracted from raw data by using MassLynx v4.2 software (Waters). For all targets reference samples were used to locate and verify the correct peaks on the EIC of the samples by retention time and MS2 fragment information. AUP values reported (FIG. 10C) are unit-less and are proportional to the concentration of the targets in the samples (relative quantification). AUP values can be used to calculate fold changes.

Untargeted Metabolomics Data analysis

Metabolomics datasets were evaluated in an untargeted fashion with Progenesis QI software (Nonlinear Dynamics), which aligns the ion intensity maps based on a reference dataset, followed by a peak picking on an aggregated ion intensity map. Detected ions were identified based on accurate mass, detected adduct patterns and isotope patterns by comparing with entries in the Human Metabolom Data Base (HMDB). A mass accuracy tolerance of 5 mDa was set for the searches. Fragmentation patterns were considered for the identifications of metabolites.

Preparation of RNA libraries
PolyA⁺ RNA sequencing

Total RNA from biological triplicates of >60-day-old Nudt2^{+/+} wild type and Nudt2 knockout (KO) mice were used for the experiment with mouse liver, spleen, pancreas, and testis samples. The tissues were isolated from dissected animals and snap-frozen in liquid nitrogen.

For RNA extraction (8), approximately 0.5 g tissue was taken and placed in a 50 mL conical tube with 5 mL of extraction buffer. [Preparation of the extraction buffer: 250 g guanidium thiocyanate, 17.6 mL sodium citrate, 0.75 M, pH 7.0 and 320 mL water were mixed at 60° C. Add 1/10 volume of sodium acetate, 2 M, pH 4.0; 1/100 volume β-mercaptoethanol, before use.] Homogenize for 20 s, and then add 5 mL phenol-H2O, mix well and stand on ice. Add 2 mL CHCl3: isoamyl alcohol (49:1), mix well, stand on ice for 15 min, and transfer all the solution to a 15 mL falcon tube. Spin down for 20 min at 4000 rpm at 4° C., transfer the upper phase (approximately 5 ml) to a new 15 mL falcon tube, and 1 mL phenol: chloroform: isoamyl alcohol (24:24:2), mix well. Spin down for 15 min at 4000 rpm at 4° C., transfer upper phase (approximately 4 ml) to a new 15 mL falcon tube, add 4 mL isopropanol, and leave it at −20° C. for 1 hour. After this, spin down at 4500 rpm for 30 min at 4° C., remove all the solution, and completely dry the tube. After, add 6 mL 4M lithium chloride to suspend the precipitate, leave on ice for 5 min, and spin down at 4500 rpm for 15 min at 4° C., remove all the solution and dry the tube completely. Add 7 mL 75% ethanol to suspend the precipitate, leave at room temperature for 10 min, spin down at 4500 rpm for 15 min, remove all the solution and dry the tube completely. Repeat the 75% ethanol step once again. Dissolve the RNA precipitation with TE buffer to a proper concentration.

The TruSeq Stranded Total RNA kit with Ribo-Zero Gold (Illumina, Cat. No. 20020612) was used for library preparation with 500 ng of total RNA as input. Library molarity and quality was assessed with the Qubit and Tapestation using a DNA High sensitivity chip (Agilent Technologies). Libraries were diluted at 2 nM and pooled before the clustering process on a HiSeq 4000 Single Read flow cell. Reads of 50 bases were generated using the TruSeq SBS reagents on the Illumina HiSeq 4000 sequencer (iGE3 Genomics Platform, University of Geneva).

All sequencing libraries prepared are listed in Table 1.

### Analysis of Deep-Sequencing Libraries

RNAseq analysis was done as previously performed (2).

### REFERENCES

1. Y. Furuichi et al., Methylated, blocked 5 termini in HeLa cell mRNA. Proceedings of the National Academy of Sciences 72, 1904-1908 (1975).
2. S. Daffis et al., 2′-O methylation of the viral mRNA cap evades host restriction by IFIT family members. Nature 468, 452-456 (2010).
3. J. G. Bird et al., Highly efficient 5′ capping of mitochondrial RNA with NAD (+) and NADH by yeast and human mitochondrial RNA polymerase. Elife 7, (2018).
4. X. Jiao et al., 5′ End Nicotinamide Adenine Dinucleotide Cap in Human Cells Promotes RNA Decay through DXO-Mediated deNADding. Cell 168, 1015-1027 e1010 (2017).
5. H. Cahova, M. L. Winz, K. Hofer, G. Nubel, A. Jaschke, NAD captureSeq indicates NAD as a bacterial cap for a subset of regulatory RNAs. Nature 519, 374-377 (2015).
6. A. G. McLennan, The Nudix hydrolase superfamily. Cell Mol Life Sci 63, 123-143 (2006).
7. M. G. Song, S. Bail, M. Kiledjian, Multiple Nudix family proteins possess mRNA decapping activity. RNA 19, 390-399 (2013).
8. R.-M. Li, M.-N. Zhang, Q.-Y. Tang, M.-G. Song, Global deletion of the RNA decapping enzyme Dcp2 postnatally in male mice results in infertility. Biochemical and biophysical research communications 526, 512-518 (2020).
9. J. Carreras-Puigvert et al., A comprehensive structural, biochemical and biological profiling of the human NUDIX hydrolase family. Nature communications 8, 1-17 (2017).
10. E. Grudzien-Nogalska et al., Structural and mechanistic basis of mammalian Nudt12 RNA deNADding. Nat Chem Biol 15, 575-582 (2019).
11. H. Wu et al., Decapping Enzyme NUDT12 Partners with BLMH for Cytoplasmic Surveillance of NAD-Capped RNAs. Cell Rep 29, 4422-4434 e4413 (2019).
12. H. Yavuz et al., A founder nonsense variant in NUDT2 causes a recessive neurodevelopmental disorder in Saudi Arab children. Clinical Genetics 94, 393-395 (2018).
13. F. Diaz et al., Novel NUDT2 variant causes intellectual disability and polyneuropathy. Annals of clinical and translational neurology 7, 2320-2325 (2020).
14. Y. Chu, G. G. Huddleston, A. N. Clancy, R. B. Harris, T. J. Bartness, Epididymal fat is necessary for spermatogenesis, but not testosterone production or copulatory behavior. Endocrinology 151, 5669-5679 (2010).
15. I. Carmi-Levy, N. Yannay-Cohen, G. Kay, E. Razin, H. Nechushtan, Diadenosine tetraphosphate hydrolase is part of the transcriptional regulation network in immunologically activated mast cells. Mol Cell Biol 28, 5777-5784 (2008).
16. P. C. Zamecnik, M. L. Stephenson, C. M. Janeway, K. Randerath, Enzymatic synthesis of diadenosine tetraphosphate and diadenosine triphosphate with a purified lysyl-sRNA synthetase. Biochem Biophys Res Commun 24, 91-97 (1966).
17. Y. Ofir-Birin et al., Structural switch of lysyl-tRNA synthetase between translation and transcription. Molecular cell 49, 30-42 (2013).
18. H. Coste, A. Brevet, P. Plateau, S. Blanquet, Non-adenylylated bis (5′-nucleosidyl) tetraphosphates occur in Saccharomyces cerevisiae and in Escherichia coli and accumulate upon temperature shift or exposure to cadmium. Journal of Biological Chemistry 262, 12096-12103 (1987).
19. O. Hudeček et al., Dinucleoside polyphosphates act as 5′-RNA caps in bacteria. Nature communications 11, 1-11 (2020).
20. D. J. Luciano, R. Levenson-Palmer, J. G. Belasco, Stresses that Raise Np4A Levels Induce Protective Nucleoside Tetraphosphate Capping of Bacterial RNA. Mol Cell, (2019).
21. U. Muller et al., Functional role of type I and type II interferons in antiviral defense. Science 264, 1918-1921 (1994).
22. J. W. Schoggins et al., A diverse range of gene products are effectors of the type I interferon antiviral response. Nature 472, 481-485 (2011).
23. A. Masumi et al., Mycobacterium avium infection induces the resistance of the interferon-γ response in mouse spleen cells at late stages of infection. Inflammation and regeneration 36, 1-8 (2016).
24. L. B. Ivashkiv, L. T. Donlin, Regulation of type I interferon responses. Nature reviews Immunology 14, 36-49 (2014).
25. E. Kowalinski et al., Structural basis for the activation of innate immune pattern-recognition receptor RIG-I by viral RNA. Cell 147, 423-435 (2011).
26. K. J. Szretter et al., 2′-O methylation of the viral mRNA cap by West Nile virus evades ifit1-dependent and -independent mechanisms of host restriction in vivo. PLOS Pathog 8, e1002698 (2012).
27. A. S. Marriott et al., NUDT2 Disruption Elevates Diadenosine Tetraphosphate (Ap4A) and Down-Regulates Immune Response and Cancer Promotion Genes. PLOS One 11, e0154674 (2016).
28. K. Baruch et al., Aging-induced type I interferon response at the choroid plexus negatively affects brain function. Science 346, 89-93 (2014).
29. A. Broggi et al., Type III interferons disrupt the lung epithelial barrier upon viral recognition. Science 369, 706-712 (2020).
30. Y.-N. Lee, H. Nechushtan, N. Figov, E. Razin, The function of lysyl-tRNA synthetase and Ap4A as signaling regulators of MITF activity in FcεRI-activated mast cells. Immunity 20, 145-151 (2004).
31. T. Su et al., Deletion of histidine triad nucleotide-binding protein 1/PKC-interacting protein in mice enhances cell growth and carcinogenesis. Proceedings of the National Academy of Sciences 100, 7824-7829 (2003).
32 S. La Shu et al., Ap4A Regulates Directional Mobility and Antigen Presentation in Dendritic Cells. Iscience 16, 524-534 (2019).
33 J. Xu, Preparation, culture, and immortalization of mouse embryonic fibroblasts. Current protocols in molecular biology 70, 28.21. 21-28.21. 28 (2005).
34. T. M. Coleman, G. Wang, F. Huang, Superior 5′ homogeneity of RNA from ATP-initiated transcription under the T7 ϕ2. 5 promoter. Nucleic acids research 32, e14-e14 (2004).
35. K. L. Beaucage, S. I. Pollmann, S. M. Sims, S. J. Dixon, D. W. Holdsworth, Quantitative in vivo micro-computed tomography for assessment of age-dependent changes in murine whole-body composition. Bone Reports 5, 70-80 (2016).
36. G. Paglia et al., Ion mobility derived collision cross sections to support metabolomics applications. Anal Chem 86, 3985-3993 (2014).
37. E. E. Schmidt, U. Schibler, Cell size regulation, a mechanism that controls cellular RNA accumulation: consequences on regulation of the ubiquitous transcription factors Oct1 and NF-Y and the liver-enriched transcription factor DBP. J Cell Biol 128, 467-483 (1995).

### Example 2

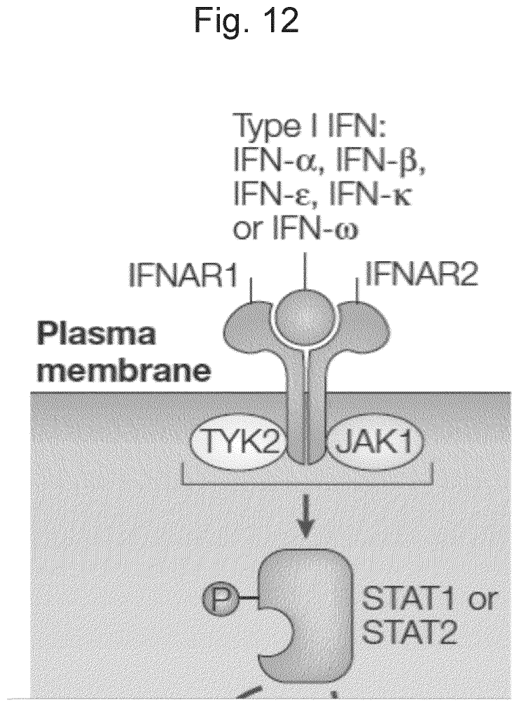

As set out above, the results of Example 1 provide a model in which NUDT2 hydrolyses Ap4A (into ATP and AMP) reducing its intracellular concentration. This prevents Ap4A-RNA generation, and thereby reduces activation the IFN pathway. This pathway is well-known in the art and shown in FIG. 12. In the IFN pathway, secreted IFN binds to interferon receptor (a heterodimer of IFNAR1 and IFNAR2) on the cell surface. The receptor transduces the signal internally via phosphorylation events catalysed by the Janus Kinase 1 (JAK1), which eventually signals to the nucleus to initiate ISG expression.

In NUDT2-deficient individuals, chronic activation of the IFN pathway is likely responsible for the observed phenotypes (including male infertility, splenomegaly, and small body size, for example).

It is rational that preventing activation of the interferon pathway reverses the phenotype seen in the Nudt2 KO mice. This may be confirmed by determining the effects of (1) deleting the interferon receptor (e.g. Ifnar1) in Nudt2 KO mice, and/or (2) treating Nudt2 KO mice with a JAK1 inhibitor such as ruxolitinib. Ruxolitinib is already approved by the FDA for an alternative indication.

### (1) Deleting the Interferon Receptor in Nudt2 KO Mice

Nudt2 KO (Nudt2−/−) females were crossed with Ifnar1 KO (B6 (Cg)-Ifnar1^{tm1.2Ees}/J; Jackson Stock No: 028288) male mice to obtain double KO mice (Nudt2−/−; Ifnar1−/−). Control animals were Nudt2−/− or Ifnar1−/− or WT.

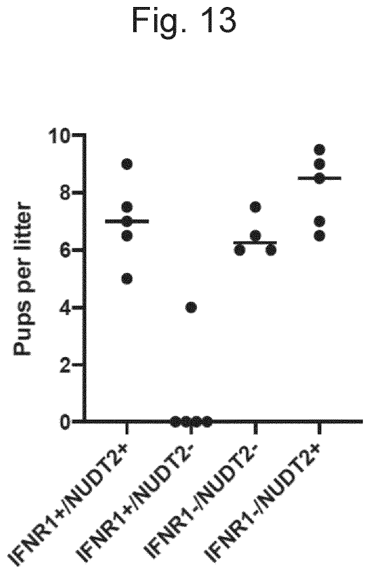

Adult males of the different genotypes were crossed with two wildtype females. Such crosses were done twice with the same males but changing the female partners. The number of pups born was counted (FIG. 13). As expected, IFNR+/NUDT2-males were observed to be sub-fertile and showed progressive loss of fertility (n=5). Interestingly, double KO males were observed to be fertile (n=4). This indicates that loss of the interferon receptor can rescue the male sub-fertility caused by Nudt2 mutation.

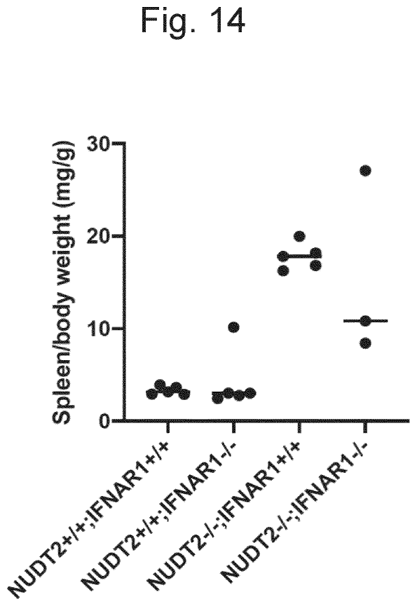

In addition, splenomegaly observed in Nudt2 KO mice is rescued by loss of the interferon receptor. Preliminary data in FIG. 14 show that double KO animals have lower spleen weight.

To further demonstrate that loss of the interferon receptor can rescue the Nudt2 KO phenotype, metabolomics and RNA mass spectrometry analyses of male and female mice are performed to confirm that the Ap4A levels and Ap4A RNA levels are still high in the double KO mice. In addition, RNAseq experiments are performed on multiple tissues to show that robust activation of the IFN pathway is prevented in double KO mice, leading to reduced ISG expression. Histological examination of testis/epididymis of male mice is also performed to confirm the absence or reduced presence of sperm in Nudt2 KO males, and the presence of increased sperm in the double KO males.

(2) Treating Nudt2 KO Mice with a JAK1 Inhibitor

Male Nudt2 KO mice are treated with a JAK1 inhibitor, such as ruxolitinib, for around one month to demonstrate that treatment rescues the Nudt2 KO phenotype. ISG expression levels in tissues of the mice are measured using RNAseq analysis, to confirm that effects of the JAK1 inhibitor are mediated via the IFN pathway.

1. A method of preventing or treating Nudt2 disorder in an individual, the method comprising administering to the individual an agent whose administration reduces type I interferon (IFN) signalling in the individual.

2. (canceled)

3. The method of claim 1, wherein the agent inhibits the signalling activity of one or more type I IFN.

4. The method of claim 3, wherein the agent inhibits a signalling pathway activated by IFNAR IFN-α receptor (IFNAR), or inhibits binding of the type I IFN to IFNAR.

5. The method of claim 4, wherein the agent inhibits the JAK-STAT signalling pathway.

6. The method of claim 1, wherein the agent reduces the amount of one or more type I IFN in the serum of the individual.

7. The method of claim 6, wherein the agent promotes hydrolysis of diadenosine tetraphosphate (Ap4A) and/or decapping of Ap4A-capped RNA.

8. The method of claim 7, wherein the agent comprises a polynucleotide sequence that encodes an enzyme that promotes hydrolysis of Ap4A and/or decapping of Ap4A-capped RNA.

9. The method of claim 8, wherein the enzyme is wild type NUDT2, or a variant thereof that retains hydrolase activity.

10. An animal model of Nudt2 disorder, wherein the animal lacks expression of functional NUDT2 protein.

11. The animal model of claim 10, wherein the animal comprises one or more non-functional Nudt2 alleles.

12. The animal model of claim 11, wherein the non-functional Nudt2 allele comprises deletion of all or part of exon 3.

13. The animal model of claim 10, wherein the animal is a rodent.

14. A method of diagnosing a Nudix disorder in an individual, comprising identifying an increased amount of substrate for a Nudix enzyme in a sample obtained from the individual, relative to a reference value.

15. The method of claim 14, wherein the Nudix disorder is Nudt2 disorder and the substrate is Ap4A or Ap4A-capped RNA, optionally wherein
(a) the amount of Ap4A is increased by about 3 to 12 fold compared to the reference value, or
(b) the amount of Ap4A-capped RNA is increased by about 5 to 15 fold compared to the reference value.

16. The method of claim 5, wherein the agent is ruxolitinib.

17. The animal model of claim 10, wherein the animal lacks expression of NUDT2 protein.

18. The animal model of claim 12, wherein the deletion is in the Nudix hydrolase domain.

19. The animal model of claim 18, wherein the deletion removes the Nudix box motif.

20. The animal model of claim 13, wherein the animal is a mouse.

21. The method of claim 14, wherein the reference value reflects the amount of substrate in a sample obtained from a healthy individual.


SEQUENCE LISTING (56 sequences)

SEQ ID NO: 1 - DNA, 929 bp, Mus musculus
  5'UTR 1..40
  misc_difference 183..249: note = Sequence deleted in mouse of Examples
  3'UTR 485..929
  source 1..929: mol_type = other DNA
gttcgaggaccgcctcctgcctctgggctacggatagatcatggccctgagggcatgtggcttgatcatcttccgaagacaccttattcccaagatggacaacagtacaattgaatttctgctgctgcaggcatcagacggcattcatcactggactcctcccaaaggccatgtggacccaggagagaatgacttagaaacagccctgcgagagactcgggaggaaacaggcatagaagcaagccaactgaccatcattgaggggttcagaagggagctcaattacgtggccaggcagaagcctaaaacggtcatttactggctggcagaagtgaaagactacaatgtagagatccgcctctcccaggagcatcaagcctaccgctggctggggctggaggaggcctgccagttggctcagttcaaggagatgaaggcaacactccaagaaggacaccagtttctctgctccacaccggcctgagctgaccagaacaaatgcttccattgcatccttaagggtttttcaagataaacccacccttccatccagggaaggatgcacgggtcatggcttgttaacaaccaagagcagataggttagtgaaattaaaattaactcggcactctcaggtgacagcaaggaagccctggattcaatccctagcatagaaaataatgactttaaaaagtaaagcaaggattttactttaaaataattactttcaaagcaagaattttagctaagttaaaaaacaaaggaggggtacggttttttaaatgacagccccgaagtatgtcttttgtcatttataaataaactggaagcaacgtacatgccctttcaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 2 - DNA, 377 bp, synthetic construct
  misc_feature 1..377: note = Coding sequence of the NUDT2 allele in the NUDT2 knockout mousedescribed in the Examples
  source 1..377: mol_type = other DNA
atggccctgagggcatgtggcttgatcatcttccgaagacaccttattcccaagatggacaacagtacaattgaatttctgctgctgcaggcatcagacggcattcatcactggactcctcccaaaggccatgtggacccaggaccatcattgaggggttcagaagggagctcaattacgtggccaggcagaagcctaaaacggtcatttactggctggcagaagtgaaagactacaatgtagagatccgcctctcccaggagcatcaagcctaccgctggctggggctggaggaggcctgccagttggctcagttcaaggagatgaaggcaacactccaagaaggacaccagtttctctgctccacaccggcctga

SEQ ID NO: 3 - DNA, 67 bp, synthetic construct
  misc_feature 1..67: note = 67bp deleted from Nudt2 in the NUDT2 knockout mouse described inthe Examples
  source 1..67: mol_type = other DNA
gagagaatgacttagaaacagccctgcgagagactcgggaggaaacaggcatagaagcaagccaact

SEQ ID NO: 4 - DNA, 98 bp, Mus musculus
  source 1..98: mol_type = genomic DNA
tggacccaggagagaatgacttagaaacagccctgcgagactcgggaggaaacaggcatagaagcaagccaactgaccatcattgaggggttcagaag

SEQ ID NO: 5 - protein, 32 aa, Mus musculus
DPGENDLETALRETREETGIEASQLTIIEGFR

SEQ ID NO: 6 - DNA, 33 bp, synthetic construct
  misc_feature 1..33: note = Sequence within the hydrolase (catalytic) domain of the Nudix boxin exon 3 of Nudt2 (SEQ ID NO: 4) following deletion of SEQ IDNO: 3
  source 1..33: mol_type = other DNA
tggacccaggaccatcattgaggggttcagaag

SEQ ID NO: 7 - protein, 30 aa, synthetic construct
  REGION 1..30: note = Amino acid sequence encoded by SEQ ID NO: 6
DPGPSLRGSEGSSITWPGRSLKRSFTGWQK

SEQ ID NO: 8 - DNA, 20 bp, synthetic construct
  misc_feature 1..20: note = gRNA1used in the preparation of the NUDT2 knockout mousedescribed in the Examples
  source 1..20: mol_type = other DNA
tcctctcttactgaatcttt

SEQ ID NO: 9 - DNA, 19 bp, synthetic construct
  misc_feature 1..19: note = gRNA2used in the preparation of the NUDT2 knockout mousedescribed in the Examples
  source 1..19: mol_type = other DNA
tgactggtagtaactccca

SEQ ID NO: 10 - DNA, 64 bp, synthetic construct
  misc_feature 1..64: note = WH-Oligo30
  source 1..64: mol_type = other DNA
gaaattaatacgactcactataggtttctaagtcattctctcctgttttagagctagaaatagc

SEQ ID NO: 11 - DNA, 64 bp, synthetic construct
  misc_feature 1..64: note = WH-Oligo31
  source 1..64: mol_type = other DNA
gaaattaatacgactcactataggacccctcaatgatggtcagtgttttagagctagaaatagc

SEQ ID NO: 12 - DNA, 80 bp, synthetic construct
  misc_feature 1..80: note = WH-Oligo60
  source 1..80: mol_type = other DNA
aaaagcaccgactcggtgccactttttcaagttgataacggactagccttattttaacttgctatttctagctctaaaac

SEQ ID NO: 13 - DNA, 23 bp, synthetic construct
  misc_feature 1..23: note = WH-Oligo98
  source 1..23: mol_type = other DNA
tattaaccatctccagaggaaag

SEQ ID NO: 14 - DNA, 20 bp, synthetic construct
  misc_feature 1..20: note = WH-Oligo100
  source 1..20: mol_type = other DNA
acttctgccagccagtaaat

SEQ ID NO: 15 - DNA, 70 bp, synthetic construct
  misc_feature 1..70: note = WH-Oligo-421
  source 1..70: mol_type = other DNA
cagtaatacgactcactattagtccgtccgccgcccttgctggtctttctcgcccgctgctgcctgcctg

SEQ ID NO: 16 - DNA, 70 bp, synthetic construct
  misc_feature 1..70: note = WH-Oligo-424
  source 1..70: mol_type = other DNA
caggcaggcagcagcgggcgagaaagaccagcaagggcggcggacggactaatagtgagtcgtattactg

SEQ ID NO: 17 - DNA, 26 bp, synthetic construct
  misc_feature 1..26: note = WH-Oligo-297
  source 1..26: mol_type = other DNA
cagtaatacgactcactattagtccg

SEQ ID NO: 18 - DNA, 19 bp, synthetic construct
  misc_feature 1..19: note = WH-Oligo-298
  source 1..19: mol_type = other DNA
aaacccaccaaaggcagac

SEQ ID NO: 19 - DNA, 27 bp, synthetic construct
  misc_feature 1..27: note = WH-Oligo48
  source 1..27: mol_type = other DNA
gctctagaatggccttgagagcatgtg

SEQ ID NO: 20 - DNA, 32 bp, synthetic construct
  misc_feature 1..32: note = WH-Oligo49
  source 1..32: mol_type = other DNA
atttgcggccgctcaggcctctatggagcaaa

SEQ ID NO: 21 - DNA, 26 bp, synthetic construct
  misc_feature 1..26: note = WH-Oligo-413
  source 1..26: mol_type = other DNA
aaactcgagatggcggccgtgcaggc

SEQ ID NO: 22 - DNA, 37 bp, synthetic construct
  misc_feature 1..37: note = WH-Oligo-414
  source 1..37: mol_type = other DNA
aaagcggccgcctagacagaagtgccaactgttgtgc

SEQ ID NO: 23 - DNA, 35 bp, synthetic construct
  misc_feature 1..35: note = WH-Oligo-401
  source 1..35: mol_type = other DNA
gcagacccaacagcaagcaggcatagaagcaggcc

SEQ ID NO: 24 - DNA, 35 bp, synthetic construct
  misc_feature 1..35: note = WH-Oligo-402
  source 1..35: mol_type = other DNA
tgctgttgggtctgcctcagggctgtttccaagtc

SEQ ID NO: 25 - DNA, 19 bp, synthetic construct
  misc_feature 1..19: note = WH-Oligo-436
  source 1..19: mol_type = other DNA
cttcgctctgtgctcgtgt

SEQ ID NO: 26 - DNA, 20 bp, synthetic construct
  misc_feature 1..20: note = WH-Oligo-437
  source 1..20: mol_type = other DNA
gaacaggaaagcaccacgga

SEQ ID NO: 27 - DNA, 19 bp, synthetic construct
  misc_feature 1..19: note = WH-Oligo-364
  source 1..19: mol_type = other DNA
gttgtctcctgcgacttca

SEQ ID NO: 28 - DNA, 19 bp, synthetic construct
  misc_feature 1..19: note = WH-Oligo-365
  source 1..19: mol_type = other DNA
ggtggtccagggtttctta

SEQ ID NO: 29 - DNA, 64 bp, synthetic construct
  misc_feature 1..64: note = WH-Oligo27
  source 1..64: mol_type = other DNA
gaaattaatacgactcactataggggacgcgcaggacagctgccgttttagagctagaaatagc

SEQ ID NO: 30 - DNA, 64 bp, synthetic construct
  misc_feature 1..64: note = WH-Oligo28
  source 1..64: mol_type = other DNA
gaaattaatacgactcactatagggtcagagcgttcaacgcggagttttagagctagaaatagc

SEQ ID NO: 31 - DNA, 18 bp, synthetic construct
  misc_feature 1..18: note = WH-Oligo95
  source 1..18: mol_type = other DNA
gatgcagatgcgctttga

SEQ ID NO: 32 - DNA, 16 bp, synthetic construct
  misc_feature 1..16: note = WH-Oligo97
  source 1..16: mol_type = other DNA
ctccagcccatggtcc

SEQ ID NO: 33 - DNA, 64 bp, synthetic construct
  misc_feature 1..64: note = WH-Oligo33
  source 1..64: mol_type = other DNA
gaaattaatacgactcactataggcgatggattgtccccggagggttttagagctagaaatagc

SEQ ID NO: 34 - DNA, 64 bp, synthetic construct
  misc_feature 1..64: note = WH-Oligo34
  source 1..64: mol_type = other DNA
gaaattaatacgactcactataggcgtgctgatgacctgtcctggttttagagctagaaatagc

SEQ ID NO: 35 - DNA, 20 bp, synthetic construct
  misc_feature 1..20: note = WH-Oligo101
  source 1..20: mol_type = other DNA
gttcttagttactgtggtgc

SEQ ID NO: 36 - DNA, 19 bp, synthetic construct
  misc_feature 1..19: note = WH-Oligo103
  source 1..19: mol_type = other DNA
tgtctttcagggtgacgag

SEQ ID NO: 37 - DNA, 64 bp, synthetic construct
  misc_feature 1..64: note = WH-Oligo36
  source 1..64: mol_type = other DNA
gaaattaatacgactcactataggagacaatagaagatgctgtggttttagagctagaaatagc

SEQ ID NO: 38 - DNA, 64 bp, synthetic construct
  misc_feature 1..64: note = WH-Oligo37
  source 1..64: mol_type = other DNA
gaaattaatacgactcactataggagtatgtctcttgtcaaccagttttagagctagaaatagc

SEQ ID NO: 39 - DNA, 19 bp, synthetic construct
  misc_feature 1..19: note = WH-Oligo104
  source 1..19: mol_type = other DNA
attgaatgggggctctttc

SEQ ID NO: 40 - DNA, 32 bp, synthetic construct
  misc_feature 1..32: note = WH-Oligo106
  source 1..32: mol_type = other DNA
ctatttcattcttgtcaactttaatttctgta

SEQ ID NO: 41 - DNA, 64 bp, synthetic construct
  misc_feature 1..64: note = WH-Oligo39
  source 1..64: mol_type = other DNA
gaaattaatacgactcactatagggcattcttcccaggtctcacgttttagagctagaaatagc

SEQ ID NO: 42 - DNA, 64 bp, synthetic construct
  misc_feature 1..64: note = WH-Oligo40
  source 1..64: mol_type = other DNA
gaaattaatacgactcactataggaaatgtgtgctttgcctccggttttagagctagaaatagc

SEQ ID NO: 43 - DNA, 29 bp, synthetic construct
  misc_feature 1..29: note = WH-Oligo107
  source 1..29: mol_type = other DNA
cagtctaaagcataaactaattaccattg

SEQ ID NO: 44 - DNA, 17 bp, synthetic construct
  misc_feature 1..17: note = WH-Oligo109
  source 1..17: mol_type = other DNA
cctcggctccgaatcat

SEQ ID NO: 45 - DNA, 64 bp, synthetic construct
  misc_feature 1..64: note = WH-Oligo42
  source 1..64: mol_type = other DNA
gaaattaatacgactcactatagggacttccagatactggagtggttttagagctagaaatagc

SEQ ID NO: 46 - DNA, 64 bp, synthetic construct
  misc_feature 1..64: note = WH-Oligo43
  source 1..64: mol_type = other DNA
gaaattaatacgactcactataggttgtgtccttctggggttatgttttagagctagaaatagc

SEQ ID NO: 47 - DNA, 16 bp, synthetic construct
  misc_feature 1..16: note = WH-Oligo110
  source 1..16: mol_type = other DNA
tcaccttgggtgtggg

SEQ ID NO: 48 - DNA, 18 bp, synthetic construct
  misc_feature 1..18: note = WH-Oligo112
  source 1..18: mol_type = other DNA
atgacttggggactccag

SEQ ID NO: 49 - DNA, 64 bp, synthetic construct
  misc_feature 1..64: note = WH-Oligo45
  source 1..64: mol_type = other DNA
gaaattaatacgactcactataggcgtcatcgggcagcgccgcggttttagagctagaaatagc

SEQ ID NO: 50 - DNA, 64 bp, synthetic construct
  misc_feature 1..64: note = WH-Oligo46
  source 1..64: mol_type = other DNA
gaaattaatacgactcactatagggctcctgcggccgcgggacggttttagagctagaaatagc

SEQ ID NO: 51 - DNA, 19 bp, synthetic construct
  misc_feature 1..19: note = WH-Oligo113
  source 1..19: mol_type = other DNA
gagccccctcggcaacccc

SEQ ID NO: 52 - DNA, 20 bp, synthetic construct
  misc_feature 1..20: note = WH-Oligo115
  source 1..20: mol_type = other DNA
caggccggccggaggcgaca

SEQ ID NO: 53 - DNA, 64 bp, synthetic construct
  misc_feature 1..64: note = Forward (F) primer design template used in the Examples.
  misc_difference 25..34: note = n represents gene specific sequence
  misc_difference 35..44: note = n is a, c, g, or t
  source 1..64: mol_type = other DNA
gaaattaatacgactcactataggnnnnnnnnnnnnnnnnnnnngttttagagctagaaatagc

SEQ ID NO: 54 - DNA, 108 bp, synthetic construct
  misc_feature 1..108: note = Sequence of the N-terminal 3xFLAG-HA tag
  misc_feature 70..81: note = spacer
  source 1..108: mol_type = other DNA
atggactacaaagaccatgacggtgattataaagatcatgatatcgattacaaggatgacgatgacaagggcggcagcggctacccatatgatgttccagattacgct

SEQ ID NO: 55 - DNA, 220 bp, synthetic construct
  misc_feature 1..220: note = DNA template used in the Examples
  regulatory 1..20: note = T7 phi 2.5 promoter; regulatory_class = promoter
  misc_feature 21: note = Transcripton start site adenosine
  source 1..220: mol_type = other DNA
cagtaatacgactcactattagtccgtccgccgcccttgctggtctttctcgcccgctgctgcctgcctgcgcttcgctctgtgctcgtgtcccgtcgcgttgggctccctggcttctgggtttccttcttggtgcgtgcgcctccggtggtgtgctcgttccgtggtgctttcctgttctcgggtcgcggtgtttgttcgggtctgcctttggtgggtt

SEQ ID NO: 56 - DNA, 70 bp, synthetic construct; identical to SEQ ID NO: 15
  misc_feature 1..70: note = DNA template used in the Examples
  regulatory 1..20: note = T7 phi 2.5 promoter; regulatory_class = promoter
  misc_feature 21: note = Transcription start site adenosine
  source 1..70: mol_type = other DNA